SEQUENCE LISTING (17 sequences)

SEQ ID NO: 1 - protein, 120 aa, Artificial Sequence
  HEL4 heavy chain variable (VH) region
QVQLLESGGGLVQPGGSLRLSCAASGFRISDEDMGWVRQAPGKGLEWVSSIYGPSGSTYYADSVKGRFTISRDNSKNTLYLQMNSLRAEDTAVYYCASALEPLSEPLGFWGQGTLVTVSS

SEQ ID NO: 2 - protein, 116 aa, Artificial Sequence
  DP47 heavy chain variable (VH) region
QVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSS

SEQ ID NO: 3 - protein, 108 aa, Artificial Sequence
  Light chain variable (VL) chain
DIQMTQSPSSLSASVGDRVTITCRASQSISSYLNWYQQKPGKAPKLLIYAYSDLQSGVPSGFSGSGSGTDFTLTISSLQPEDFATYYCQQLMTHPTTFGQGTKVEIKR

SEQ ID NO: 4 - protein, 16 aa, Artificial Sequence
  Linker sequence between VH and VL domains
GGGGSGGGGSGGGGST

SEQ ID NO: 5 - protein, 120 aa, Artificial Sequence
  Heavy chain variable (VH) region VHhTNF_G07 (anti-hTNF VH)
QVQLLESGGGLVQPGGSLRLSCAASGFRISDEDMGWVRQAPGKGLEWVSSIYGPSGSTYYADSVKGRFTISRDNSKNTLYLQMNSLRAEDTAVYYCASKEDYDTYNWGFWGQGTLVTVSS

SEQ ID NO: 6 - protein, 120 aa, Artificial Sequence
  Heavy chain variable (VH) region VHmIL21_G11 (anti-mIL21 VH)
QVQLLESGGGLVQPGGSLRLSCAASGFRISDEDMGWVRQAPGKGLEWVSSIYGPSGSTYYADSVKGRFTISRDNSKNTLYLQMNSLRAEDTAVYYCASASNTYEDMLGFWGQGTLVTVSS

SEQ ID NO: 7 - protein, 121 aa, artificial
  Heavy chain variable (VH) region VHPRLR_C02 (anti-hPRLR VH)
EVQLLESGGGLVQPGGSLRLSCAASGFRFADEDMGWVRQAPGKGLEWVSDISGDGYSTYYADSVKGRFTISRDNSKNTLYLQMNSLRAEDTAVYYCAKLPPVYLGGAFDYWGQGTLVTVSS

SEQ ID NO: 8 - protein, 121 aa, artificial
  Heavy chain variable (VH) region VHHEL_H04 (anti-HEL VH)
EVQLLESGGGLVQPGGSLRLSCAASGFRFDAEDMGWVRQAPGKGLEWVSSIYGPSGSTYYADSVKGRFTISRDNSKNTLYLQMNSLRAEDTAVYYCAKYTSPPQNHGFDYWGQGTLVTVSS

SEQ ID NO: 9 - protein, 121 aa, artificial
  Heavy chain variable (VH) region VHHEL_H08 (anti-HEL VH)
EVQLLESGGGLVQPGGSLRLSCAASGFRISDEDMGWVRQAPGKGLEWVSYIDNNGYSTYYADSVKGRFTISRDNSKNTLYLQMNSLRAEDTAVYYCAKKQYGYQNGGFDYWGQGTLVTVSS

SEQ ID NO: 10 - protein, 121 aa, Artificial Sequence [sequence omitted]

SEQ ID NO: 11 - protein, 121 aa, Artificial Sequence [sequence omitted]

SEQ ID NO: 12 - protein, 120 aa, Artificial Sequence [sequence omitted]

SEQ ID NO: 13 - protein, 123 aa, Artificial Sequence [sequence omitted]

SEQ ID NO: 14 - DNA, 360 bp, Artificial Sequence
  Nucleic acid sequence encoding HEL4 heavy chain variable (VH) region
caggtgcagctgttggagtctgggggaggcttggtacagcctggggggtccctgcgtctctcctgtgcagcctccggatttaggattagcgatgaggatatgggctgggtccgccaggctccagggaagggtctagagtgggtatcaagcatttatggccctagcggtagcacatactacgcagactccgtgaagggccggttcaccatctcccgtgacaattccaagaacacgctgtatctgcaaatgaacagcctgcgtgccgaggacaccgcggtatattattgcgcgagtgctttggagccgctttcggagcccctgggcttttggggtcagggaaccctggtcaccgtctcgagc

SEQ ID NO: 15 - DNA, 348 bp, Artificial Sequence
  Nucleic acid sequence encoding DP47 heavy chain variable (VH) region
caggtgcagctgttggagtctgggggaggcttggtacagcctggggggtccctgcgtctctcctgtgcagcctccggattcacctttagcagctatgccatgagctgggtccgccaggctccagggaagggtctagagtgggtctcagctattagtggtagtggtggtagcacatactacgcagactccgtgaagggccggttcaccatctcccgcgacaattccaagaacacgctgtatctgcaaatgaacagcctgcgtgccgaggacaccgcggtatattactgtgcgaaaagttatggtgcttttgactactggggtcagggaaccctggtcaccgtctcgagc

SEQ ID NO: 16 - DNA, 27 bp, artificial
  Nucleotide sequence of oligonucleotide for amplifying VH
acgcgtcgacgcaggtgcagctgttgg

SEQ ID NO: 17 - DNA, 30 bp, artificial
  Nucleotide sequence of oligonucleotide for amplifying VH
ctgttaggatccgctcgagacggtgaccag

(19) United States
(12) Patent Application Publication
(10) Pub. No.: US 2016/0145328 A1
(43) Pub. Date: May 26, 2016

(54) Modified Variable Domain Molecules And Methods For Producing And Using Same

(71) Applicant: Garvan Institute of Medical Research, Darlinghurst (AU)

(72) Inventors: Daniel Christ, Darlinghurst (AU); Kip Dudgeon, Darlinghurst (AU)

(21) Appl. No.: 14/918,636

(22) Filed: Oct. 21, 2015

Related U.S. Application Data
(63) Continuation of application No. 13/502,674, filed on Apr. 18, 2012, now abandoned.
(60) Provisional application No. 61/254,460, filed on Oct. 23, 2009.

(30) Foreign Application Priority Data
Sep. 7, 2010 (AU) 2010904025

(51) Int. Cl. C07K 16/22; A61K 49/00; A61K 47/48; G01N 33/68

(52) U.S. Cl. CPC C07K 16/22; G01N 33/6893; A61K 49/0002; A61K 47/48546; G01N 2800/52; C07K 2317/92; C07K 2317/21; C07K 2317/24; C07K 2317/569; C07K 2317/622; C07K 2317/626; C07K 2319/00

(57) ABSTRACT

The present disclosure provides an isolated protein comprising an antibody heavy chain variable region (V_{H}) comprising a negatively charged amino acid at position 28 and/or 31 and/or 32 and/or 33 and/or 35 according to the numbering system of Kabat, the protein capable of binding specifically to an antigen.

### INCORPORATION BY REFERENCE

This application claims priority from U.S. Ser. No. 61/254,460 entitled “Modified variable domain molecules and methods for producing and using same” filed 23 Oct. 2009 and from Australian Provisional Patent Application No. 2010904025 entitled “Modified variable domain molecules and methods for producing and using same 2” filed 7 Sep. 2010, the entire contents of which are hereby incorporated by reference.

### FIELD

The present disclosure relates to proteins comprising an aggregation-resistant antibody variable domain and uses thereof.

### BACKGROUND

Antibodies and proteins comprising antigen binding domains are now widely used as research reagents, diagnostic/prognostic reagents, industrial reagents and therapeutic agents. This broad ranging applicability arises from the ability of antibodies and proteins comprising antigen binding domains thereof to bind to an antigen with a high degree of specificity and affinity. Accordingly, antibodies and proteins comprising antigen binding domains thereof are able to bind specifically to an antigen in a sample and permit detection, quantification or to kill the cell expressing the antigen or to deliver a therapeutic payload. However, despite their versatility, only a subset of antibodies has the biophysical properties suited for diagnostic/prognostic/industrial/therapeutic application. For example, therapeutic or in vivo diagnostic antibodies/proteins require a long serum half-life in a subject to accumulate at the desired target, and they must therefore be resistant to aggregation (Willuda et al., 1999). Industrial applications often require antibodies/proteins that have a long half-life or can function following exposure to harsh conditions, e.g., high temperatures without aggregation (Harris, 1999). Aggregation of proteins comprising antibody variable domains can lead to difficulties in expression and/or purification, immunogenicity, toxicity, degradation, impaired avidity, or loss of activity following storage.

Protein aggregation is a process that competes with the folding pathway or can arise from intermediates in the folding pathway, and usually involves association of unfolded protein or partially unfolded protein. Resistance to aggregation can be achieved by stabilizing the native state (i.e., resisting unfolding) or by reducing the propensity of the unfolded or partially folded states of the protein to aggregate. A disadvantage of stabilizing the native state is that proteins will likely be exposed to an environment in which they will unfold. Generally, when a protein is denatured or unfolds, amino acid residues that normally mediate intramolecular contacts in the interior of the protein are exposed. Such exposure often makes proteins prone to form intermolecular contacts and aggregate. In contrast to proteins that resist unfolding, a protein having a reduced propensity to aggregate when unfolded will simply refold into a bioactive non-aggregated state after exposure to such an environment.

The aggregation-resistance or aggregation-propensity of antibodies and proteins comprising antigen binding domains thereof is usually limited by the most aggregation prone domain(s) contained therein and by the strength of its interaction with surrounding domains (if present). This is because once that domain unfolds, if it is incapable of refolding it may interact with other domains in the same protein or in other proteins and form aggregates. Constant domains of antibodies generally do not aggregate and do not vary considerably in sequence (as suggested by their name). Accordingly, the weakest domains of an antibody are generally considered to be those regions that vary from one antibody to the next, i.e., variable regions (e.g., heavy chain variable region (V_{H}) and/or light chain variable region (V_{L})) (Ewert et al., 2003). In this regard, incorporation of aggregation prone scFv molecules into otherwise stable recombinant antibody products often imparts these generally undesirable traits to the new recombinant design. As stated in Ewert et al., 2008, “to improve any sub-optimal antibody construct by rational engineering, the “weakest link” has to be identified and improved”. Ewert et al., also highlights that the variable domain is generally the “weakest link” in an antibody or antibody-related molecule. Thus, engineering a variable domain to be aggregation-resistant is most likely to render the entire protein comprising that variable domain aggregation-resistant. Hoyer et al., 2002 also established that V_{H} domains have a significant effect on refolding of proteins comprising antibody variable domains. The authors conclude that V_{H}s should be a major target for modifications to improve proteins.

Various strategies have been proposed for reducing aggregation of variable domains, e.g., rational design of aggregation-resistant proteins, complementarity determining region (CDR) grafting, or introducing disulfide bonds into a variable domain.

Rational design of aggregation-resistant proteins generally involves using in silico analysis to predict the effect of a point mutation on the aggregation propensity of a protein. However, there are several difficulties with this approach. For example, it is not sufficient to merely identify a mutation that is likely to reduce aggregation of an unfolded protein. Rather, the mutation must also not increase aggregation of a folded protein or affect the function of the folded protein. Furthermore, rational design requires detailed structural analysis of the specific protein being improved and thus, is difficult to use with a protein that has not been thoroughly characterized and is not readily applicable to a variety of different proteins.

CDR grafting involves transplanting CDRs from one variable domain onto framework regions (FRs) of another variable domain. This strategy was shown to be useful in stabilizing an anti-EGP-2 scFv (Willuda et al., 1999). However, this strategy is generally used to produce variable domains that resist unfolding, which as discussed above is not the most desirable form of protein. Disadvantages of this approach include the reduction in affinity that can occur following CDR grafting. This loss of affinity can be overcome by introducing mutations to the FRs, however such mutations can produce immunogenic epitopes in the protein, thereby making the protein undesirable from a therapeutic point of view. Furthermore, CDR grafting generally requires analysis of crystal structure or homology modeling of the donor and acceptor variable regions to assess suitability for grafting. Clearly, such an approach is laborious and requires specialized knowledge. Moreover, since each variable region has a different structure, the method is not readily applied across a variety of molecules.

As for methods involving introducing disulfide bonds into a variable region, while the bond may assist in the protein correctly refolding, it also introduces rigidity into the variable domain. Such rigidity can reduce the affinity of an antibody for an antigen. Moreover, not all variable domains can support the introduction of the requisite cysteine residues for disulfide bond formation without loss of affinity or without introducing an immunogenic epitope. Furthermore, formation of disulfide bonds under high protein concentrations can lead to protein aggregation, thus negating any potential positive effect of the bond.

As will be apparent from the foregoing, there is a need in the art for aggregation-resistant variable domain containing proteins and processes for their production. Preferably, the processes are readily applicable to a variety of distinct variable regions.

### SUMMARY

In work leading up to the present invention, the inventors sought to identify amino acid residues in a variable region of an antibody that conferred resistance to aggregation, e.g., following exposure to heat. Such aggregation-resistant proteins are useful for a variety of applications, e.g., therapy and/or diagnosis/prognosis. The inventors compared the sequences of an aggregation-resistant single domain antibody comprising a V_{H} to a germ line V_{H} that has the same framework sequences, but is not aggregation-resistant. Initially, the inventors identified a large number of amino acid differences in the complementarity determining regions of the aggregation-resistant V_{H} (as shown in FIG. 1). Despite the large number of differences identified, the inventors discovered that single amino acid changes conferred aggregation-resistance to a V_{H} and that combinations of only a few changes conferred the majority of the aggregation-resistance observed. These finding prompted the inventors to further investigate the effect of changes in complementarity determining regions. The inventors determined that a negatively charged amino acid at position 28 and/or 31 and/or 32 and/or 33 and/or 35 according to the Kabat numbering system is sufficient to confer considerable aggregation-resistance on a V_{H} or a protein comprising same.

The inventors additionally found that including two or more negatively charged amino acids at positions discussed above dramatically improved aggregation-resistance of a protein comprising the V_{H} compared to either a protein lacking the negatively charged residues or comprising only one negatively charged residue. As exemplified herein, the inclusion of multiple negatively charged amino acids at positions described herein confers aggregation-resistance on protein (e.g., either in solution or displayed on the surface of a phage). This effect is marked in soluble proteins (as opposed to displayed on the surface of phage). Thus, the inventors have not only determined single amino acid residues that confer aggregation-resistance, they have additionally determined that they can considerably improve that resistance by combining those residues. In this regard, the inventors have identified numerous combinations of negatively charged amino acids that confer a greater degree of aggregation-resistance than is observed with single negatively charged amino acids. The inventors did not expect that substitution of such few amino acid residues in a V_{H} would confer such a degree of aggregation-resistance. In addition to substitutions at the positions described above, the inventors also identified other changes in complementarity determining regions that confer detectable aggregation-resistance on a V_{H} (such as negatively charged amino acids at position 26 and/or 30 and/or 50 and/or 52 and/or 52a and/or 53 according to the Kabat numbering system).

Because the mutations identified by the inventors are in complementarity determining regions (CDRs) of an antibody, they are readily transferrable between different antibodies, e.g., antibodies of different classes or subclasses that comprise different framework regions. This is because antibody variable domains have been selected to accommodate sequence variation in the CDRs, whereas the framework regions generally do not significantly vary since they provide a scaffold for presenting the CDR loops.

In addition to substitutions in complementarity determining regions that confer aggregation-resistance, the inventors also identified changes in adjacent framework regions that confer detectable aggregation-resistance on a V_{H} (such as negatively charged amino acids at position 39 and/or 40 according to the Kabat numbering system).

These findings have permitted the inventors to produce several aggregation-resistant proteins comprising a V_{H} that are capable of specifically binding to an antigen in addition to libraries of such proteins useful for screening to identify new proteins comprising V_{H} domains, e.g., useful as therapeutic and/or diagnostic reagents.

Proteins produced by the inventors were also expressed at higher levels in recombinant systems compared to proteins lacking the negatively charged amino acid(s). Furthermore, by combining multiple negatively charged amino acids, the inventors were able to obtain greater levels of expression of soluble protein compared to proteins lacking the negatively charged amino acids or containing a single negatively charged amino acid residue.

Proteins produced by the inventors also showed a reduced propensity to be trapped by chromatography resins during purification, thereby increasing yield. In this regard, the inventors again showed that inclusion of a single residue as identified herein conferred a considerable advantage over a protein lacking such a residue and that inclusion of multiple negatively charged residues further improved this effect.

The inventors also found that proteins comprising negatively charged amino acid(s) as discussed above were capable of higher degrees of concentration without aggregation compared to proteins lacking such residues, providing a clear advantage for storage and for production of high concentration compositions, e.g., pharmaceutical compositions.

The aggregation-resistance of the proteins produced by the inventors also provides an advantage during purification, since the proteins can be heated to reduce the presence of dimers/trimers and then purified. This not only reduces the presence of undesirable forms of the protein but also potentially increases yield.

The inventors have additionally produced libraries of aggregation-resistant proteins and shown that they can isolate proteins therefrom that specifically bind to antigen and/or bind to antigen with high affinity. Proteins isolated from the libraries were also shown to be aggregation-resistant.

Accordingly, the present disclosure provides an isolated protein comprising an antibody V_{H} comprising a negatively charged amino acid at position 32 and/or position 33 according to the numbering system of Kabat, the protein capable of specifically binding to an antigen other than hen egg lysozyme, beta galactosidase, alpha amylase, B5R or wherein:
(i) if the protein binds to human vascular endothelial growth factor (VEGF) and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35.

Optionally, the protein additionally comprises a negatively charged amino acid at a position selected from the group consisting of position 28 and/or 31 and/or 35 according to the numbering system of Kabat. In one example, the protein additionally comprises a negatively charged amino acid at position 31 according to the numbering system of Kabat.

In an additional or alternative example, the protein comprises an aggregation-resistant V_{H}.

In one example, the protein is not HEL4 (i.e., does not comprise a sequence set forth in SEQ ID NO: 1).

The present disclosure also provides an isolated protein comprising an antibody VH comprising a negatively charged amino acid at position 28, 33 and/or 35 according to the numbering system of Kabat, the protein capable of specifically binding to an antigen other than hen egg lysozyme, beta galactosidase, alpha amylase B5R or wherein:
(i) if the protein binds to human VEGF and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35.

The present disclosure also provides an isolated protein comprising an antibody V_{H} comprising negatively charged amino acids at two or more positions selected from the group consisting of 28 and/or 31 and/or 32 and/or 33 and/or 35 according to the numbering system of Kabat, the protein capable of specifically binding to an antigen other than hen egg lysozyme, beta galactosidase, alpha amylase, B5R or wherein:
(i) if the protein binds to human VEGF and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35.

In one example, the protein comprises an aggregation-resistant V_{H}.

The present disclosure also provides an isolated protein comprising an antibody V_{H} comprising a negatively charged amino acid at position 28, 33 and/or 35 according to the numbering system of Kabat, the protein capable of specifically binding to an antigen with an affinity of more than 10 μM or 5 μM or 1 μM, preferably more than 100 nM, wherein:
(i) if the protein binds to human VEGF and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35.

In one example, the protein comprises an aggregation-resistant V_{H}.

The present disclosure also provides an isolated protein comprising an antibody V_{H} comprising negatively charged amino acids at two or more positions selected from the group consisting of 28 and/or 31 and/or 32 and/or 33 and/or 35 according to the numbering system of Kabat, the protein capable of specifically binding to an antigen with an affinity of more than 10 μM or 5 μM or 1 μM, preferably more than 100 nM, wherein:
(i) if the protein binds to human VEGF and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35.

In one example, the protein does not bind to beta galactosidase, alpha amylase, B5R, human VEGF or human tumor necrosis factor α.

The present disclosure also provides a protein comprising an antibody V_{H} capable of specifically binding to an antigen, wherein the V_{H} comprises a sequence of contiguous amino acids comprising the sequence X₁X₂X₃X₄X₅X₆X₇X₈, wherein X₁ corresponds to position 28 according to the Kabat numbering system,
wherein at least two of X₁, X₄, X₅, X₆ and X₈ are a negatively charged amino acid and the remaining amino acids at X₁-X₈ are any amino acid,
and wherein the protein does not bind to hen egg lysozyme or beta galactosidase or B5R,
and wherein:
(i) if the protein binds to human VEGF and comprises aspartic acid at positions X₅ and X₆ it comprises at least one additional negatively charged amino acid between positions X₂ and X₈; and
(ii) if the protein binds to human VEGF and comprises aspartic acid at positions X₄ and X₅ it comprises at least one additional negatively charged amino acid between positions X₁ and X₈.

In one example, the protein has reduced tendency to aggregate compared to the protein without the negatively charged amino acid discussed above. For example, the protein has reduced tendency to aggregate after heating to at least about 60° C. or 70° C. or, preferably, 80° C. compared to the protein without the negatively charged amino acid.

In one example, the protein retains the ability to specifically bind to the antigen after heating to at least about 60° C. or 70° C. or, preferably 80° C.

In one example, the protein is capable of binding to (preferably, specifically binding to) a human protein (other than human VEGF or human tumor necrosis factor α, where appropriate).

In another example, the protein is capable of binding to (preferably, specifically binding to) a protein associated with or causative of a human condition (other than VEGF or human tumor necrosis factor α, where appropriate). Such a protein can be a human protein, or a protein from, e.g., an infectious organism. Preferably, the protein is a human protein (other than VEGF or human tumor necrosis factor α where appropriate). Exemplary proteins are soluble and/or secreted proteins or receptors (e.g., extracellular domains of receptors) or membrane bound proteins (e.g., extracellular domains of membrane bound proteins).

In one example, the negatively charged amino acid is glutamic acid. In another example, the negatively charged amino acid is aspartic acid.

In one example, the negatively charged amino acid at position 28 and/or 31 and/or 33 and/or 35 is aspartic acid.

In one example, the negatively charged amino acid at position 32 is aspartic acid or glutamic acid.

In an exemplary form, the protein comprises a negatively charged amino acid at positions 32 and 33 according to the numbering system of Kabat. In another exemplary form, the protein comprises a negatively charged amino acid at positions 31 and 32 and 33 according to the numbering system of Kabat.

In another example, the protein additionally comprises a negatively charged amino acid at one or more residues selected individually or collectively from the group consisting of position 26, 30, 39, 40, 50, 52, 52a and 53 according to the numbering system of Kabat. Preferably, the negatively charged amino acid is at position 30, for example, this negatively charged amino acid is aspartic acid.

Preferably, the negatively charged amino acid is aspartic acid.

An exemplary protein as described herein comprises the following:
(i) a negatively charged amino acid at two or more residues selected individually or collectively from the group consisting of 28, 31, 32, 33 and 35 according to the numbering system of Kabat; and
(ii) optionally, a negatively charged amino acid at one or more residues selected individually or collectively from the group consisting of position 26, 30, 39, 40, 50, 52, 52a and 53 according to the numbering system of Kabat.

Another exemplary protein as described herein comprises the following:
(i) a negatively charged amino acid at positions 32 and 33 according to the numbering system of Kabat; and
(ii) optionally, a negatively charged amino acid at one or more residues selected individually or collectively from the group consisting of position 26, 28, 30, 31, 35, 39, 40, 50, 52, 52a and 53 according to the numbering system of Kabat.

In one exemplary form of the disclosure, a protein comprises negatively charged amino acids at positions 31 and 32 and 33 according to the numbering system of Kabat.

For example, the protein comprises:
(i) a glutamic acid at position 32 according to the numbering system of Kabat; and
(ii) an aspartic acid at position 33 according to the numbering system of Kabat.

For example, the protein comprises:
(i) an aspartic acid at position 31 according to the numbering system of Kabat;
(ii) a glutamic acid at position 32 according to the numbering system of Kabat; and
(iii) an aspartic acid at position 33 according to the numbering system of Kabat.

Optionally, the protein additionally comprises a negatively charged amino acid (e.g., aspartic acid) at position 28 and/or 35 according to the numbering system of Kabat.

In one example, the protein comprises a negatively charged amino acid at positions, 28, 32 and 33 or positions 28, 31, 32 and 33 or positions 32, 33 and 35 or positions 31, 32, 33 and 35 or positions 28, 31, 32, 33 and 25.

The present disclosure is also useful for producing modified forms of existing proteins that have improved aggregation-resistance. Accordingly, the present disclosure additionally provides a protein comprising a modified antibody heavy chain variable region (V_{H}) capable of specifically binding to an antigen, wherein the V_{H} comprises a negatively charged amino acid at position 31 and/or position 33 according to the numbering system of Kabat, and wherein the unmodified form of the V_{H} does not comprise the negatively charged amino acids.

The present disclosure additionally provides a protein comprising a modified antibody V_{H} capable of specifically binding to an antigen, wherein the V_{H} comprises a negatively charged amino acid at position 28, 31, 33 and/or 35 according to the numbering system of Kabat, and wherein the unmodified form of the V_{H} does not comprise the negatively charged amino acid(s). Preferably, the unmodified form of the V_{H} binds to the same antigen (e.g., same epitope) as the modified V_{H}.

The present disclosure additionally provides a protein comprising a modified V_{H} capable of specifically binding to an antigen, wherein the V_{H} comprises two or more positions selected from the group consisting of 28 and/or 31 and/or 32 and/or 33 and/or 35 according to the numbering system of Kabat, wherein the unmodified protein does not comprise the two or more negatively charged amino acids at positions 28 and/or 31 and/or 32 and/or 33 and/or 35 according to the numbering system of Kabat. Preferably, the unmodified form of the V_{H} binds to the same antigen (e.g., same epitope) as the modified V_{H}.

The present disclosure also provides a protein comprising a modified V_{H} capable of specifically binding to an antigen, wherein the V_{H} comprises a sequence of contiguous amino acids comprising the sequence X₁X₂X₃X₄X₅X₆X₇X₈, wherein X₁ corresponds to position 28 according to the Kabat numbering system,
wherein at least two of X₁, X₄, X₅, X₆ and X₈ are a negatively charged amino acid and the remaining amino acids at X₁-X₈ are any amino acid,
wherein the unmodified protein does not comprise the two or more negatively charged amino acids at positions X₁, X₄, X₅, X₆ and X₈.

In one example, the protein comprises a modified aggregation-resistant V_{H}.

In one example, the protein comprises:
(i) an aspartic acid at position 31 according to the numbering system of Kabat; and/or
(ii) a glutamic acid at position 32 according to the numbering system of Kabat; and/or
(iii) an aspartic acid at position 33 according to the numbering system of Kabat.

Optionally, the protein additionally comprises a negatively charged amino acid (e.g., aspartic acid) at position 28 and/or 35 according to the numbering system of Kabat.

Exemplary features of such a protein (e.g., additional sites for negatively charged amino acids and/or specific negatively charged amino acids) are described herein and shall be taken to apply mutatis mutandis to the present form of the disclosure.

In one example, the protein is an antibody.

In one example, the protein or antibody does not bind to hen egg lysozyme, beta galactosidase, alpha amylase, B5R (e.g., from Vaccinia virus) or wherein:
(i) if the protein binds to human VEGF and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35.

Preferably, the protein binds to a human protein, for example, a human protein associated with or causative of a disease.

In another example, a protein as described herein according to any example does not comprise a disulfide bond in a CDR, e.g., CDR3.

In another example, a variable region within the protein as described herein according to any example does not have an overall acidic isoelectric point.

Exemplary proteins of the present disclosure are human, humanized or deimmunized at amino acid positions other than 28 and/or 31 and/or 32 and/or 33 and/or 35 according to the numbering system of Kabat, or are fused to a human protein or region thereof (e.g., are chimeric antibodies).

In one example, the protein of the present disclosure is in the form of a single domain antibody (dAb) or a dAb fused to another protein (e.g., a Fc region).

In an alternative example, a protein of the present disclosure additionally comprises a light chain variable region (V_{L}), wherein the V_{H} and the V_{L} associate to form a Fv (e.g., comprising an antigen binding site). In one example, the Fv is capable of specifically binding to an antigen.

In one example, the V_{H} and the V_{L} are in different polypeptide chains. For example, the protein is in the form of an antibody, a diabody, a triabody, a tetrabody or a Fv.

In another example, the V_{H} and the V_{L} are in the same polypeptide chain. For example, the protein is in the form of a (scFv)n or a fusion protein comprising a (scFv)n, wherein n is a number between 1 and 10.

In an alternative or additional example, other than those proteins discussed above as having a specific affinity, a protein specifically binds to a target antigen or epitope with an affinity of less than 5 μM, preferably less than 1 μM, preferably less than 500 nM, preferably less than 200 nM, and more preferably less than 10 nM, such as less than 1 nM.

In an alternative or additional example, any proteins discussed herein specifically binds to a target antigen or epitope with an affinity of greater than 100 pM, preferably greater than 10 pM, preferably greater than 1 pM.

In an additional or alternative example, any protein of the present disclosure dissociates from its target antigen(s) with a K_{D} of 300 nM or less, 300 nM to 5 pM, preferably 50 nM to 20 pM, or 5 nM to 200 pM or 1 nM to 100 pM.

In one example, a protein of the present disclosure comprises a CDR1 of a protein comprising a sequence at least about 80% (or 90% or 95% or 99% or 100%) identical to a sequence set forth in SEQ ID NO: 2 modified to comprise a (or two or more) negatively charged amino acid(s) at position(s) 28 and/or 31 and/or 32 and/or 33 and/or 35 (and/or any additional site described herein). In one example, a protein of the present disclosure comprises a sequence at least about 80% (or 90% or 95% or 99% or 100%) identical to a sequence set forth in SEQ ID NO: 5, 6, 7 or comprises a CDR3 (preferably, a heavy chain CDR3) of said protein. In one example of the disclosure, a protein of the present disclosure comprises a sequence at least about 80% (or 90% or 95% or 99% or 100%) identical to a sequence set forth in any one of SEQ ID NOs: 10-13 modified to comprise a negatively charged amino acid as described herein according to any example. In one example of the disclosure, a protein of the present disclosure comprises a sequence at least about 80% (or 90% or 95% or 99% or 100%) identical to a sequence set forth in any one of SEQ ID NOs: 10-13 modified to comprise a (or two or more) negatively charged amino acid(s) at position(s) 28 and/or 31 and/or 32 and/or 33 and/or 35 (and/or any additional site described herein).

The present disclosure also provides a protein of the present disclosure conjugated to a compound. For example, the compound is selected from the group consisting of a radioisotope, a detectable label, a therapeutic compound, a colloid, a toxin, a nucleic acid, a peptide, a protein, a compound that increases the half life of the protein in a subject and mixtures thereof.

The present disclosure also provides a composition comprising a protein of the present disclosure and a pharmaceutically acceptable carrier.

The present disclosure additionally provides a nucleic acid encoding a protein of the present disclosure. In one example, the nucleic acid is in an expression construct and is operably linked to a promoter. For example, the expression construct is an expression vector.

The present disclosure also provides a cell expressing a protein of the present disclosure. For example, the cell comprises a nucleic acid or expression construct of the disclosure. Exemplary cells include mammalian cells, plant cells, fungal cells and prokaryotic cells.

The present disclosure also provides a method for producing a protein of the present disclosure, the method comprising maintaining an expression construct of the disclosure for a time and under conditions sufficient for (or such that) the encoded protein is produced. For example, the method comprises culturing a cell of the disclosure for a time and under conditions sufficient for (or such that) a protein of the present disclosure is produced.

In one example, the method additionally comprises isolating the protein of the present disclosure. In one example, the method additionally comprises heating the protein, e.g., to at least about 50° C. or 60° C. or 70° C. or 80° C. prior to, during or after isolating the protein. For example, the protein is heated to there by reduce the amount of dimers and/or trimers that naturally occur during expression and purification processes. Such a method can facilitate recovery of increased levels of protein of the present disclosure.

Optionally, the method additionally comprises conjugating the protein to a compound or formulating the compound into a pharmaceutical composition.

The present disclosure additionally provides a library comprising a plurality of proteins of the present disclosure.

The present disclosure also provides a library comprising proteins comprising antibody V_{H}s, wherein at least 30% (or 40% or 50% or 60% or 70% or 80% or 90% or 95% or 98% or 99%) of the V_{H}s comprise negatively charged amino acids at two or more positions selected from the group consisting of 28 and/or 31 and/or 32 and/or 33 and/or 35 according to the numbering system of Kabat.

The present disclosure also provides a library comprising proteins comprising antibody V_{H}s, wherein at least 30% (or 40% or 50% or 60% or 70% or 80% or 90% or 95% or 98% or 99%) of the V_{H}s comprise a sequence of contiguous amino acids comprising the sequence X₁X₂X₃X₄X₅X₆X₇X₈, wherein X₁ corresponds to position 28 according to the Kabat numbering system,
wherein at least two of X₁, X₄, X₅, X₆ and X₈ are a negatively charged amino acid and the remaining amino acids at X₁-X₈ are any amino acid

In one example, the V_{H}s comprising negatively charged amino acids comprise the residues at positions 32 and 33; or 31 and 32 and 33.

Additional sites for negatively charged amino acids, or specific negatively charged amino acids that can be included are described herein and are to be taken to apply mutatis mutandis to the present example.

In one example, the proteins are displayed on the surface of a particle (e.g., a phage or a ribosome) or a cell.

In one example, the amino acids in the CDRs (e.g., in CDR3 or in CDR2 and 3 or in CDR 1, 2 and 3) of the V_{H} domains other than those at position 32 and/or 33 (and, optionally 31) are random or semi-random or are derived from a human antibody.

In another example, the amino acids in the CDRs (e.g., in CDR3 or in CDR2 and 3 or in CDR 1, 2 and 3) of the V_{H} domains other than those at position 28 and/or 31 and/or 32 and/or 33 and/or 35 are random or semi-random or are derived from a human antibody.

Clearly, the present disclosure also provides a library of nucleic acids encoding said library.

The present disclosure additionally provides a method for isolating a protein of the present disclosure, the method comprising contacting a library of the disclosure with an antigen for a time and under conditions sufficient for (or such that) a protein binds to the antigen and isolating the protein.

The present disclosure additionally provides a method for producing a library comprising a plurality of proteins of the present disclosure, the method comprising:
(i) obtaining or producing nucleic acids encoding a plurality of proteins comprising V_{H} domains, wherein the V_{H} domains comprise a negatively charged amino acid at positions discussed above;
(ii) producing a library of expression constructs comprising the following operably linked nucleic acids:
   (a) a promoter;
   (b) a nucleic acid obtained or produced at (i); and
   (c) a nucleic acid encoding a polypeptide that facilitates display of the V_{H} containing protein in/on the cells or particles; and
(iii) expressing proteins encoded by the expression constructs such that they are displayed in/on the cells or particles.

In one example, the amino acids in the CDRs (e.g., in CDR3 or in CDR2 and 3 or in CDR 1, 2 and 3) of the V_{H} domains other than those at position 32 and/or 33 (and, optionally 31) are random or semi-random or are derived from a human antibody.

In another example, the amino acids in the CDRs (e.g., in CDR3 or in CDR2 and 3 or in CDR 1, 2 and 3) of the V_{H} domains other than those at position 28 and/or 31 and/or 32 and/or 33 and/or 35 are random or semi-random or are derived from a human antibody.

In one example, the method additionally comprises isolating nucleic acid encoding the protein. Such a nucleic acid can be introduced into an expression construct. Optionally, the protein can be expressed.

The present disclosure also contemplates modifications to the isolated proteins, such as affinity maturation and/or humanization and/or deimmunization.

Such an isolated protein can be used to produce, e.g., an antibody.

The present disclosure is also useful for reducing the aggregation propensity or increasing the aggregation-resistance of an existing antibody or protein comprising a V_{H} thereof. For example, the present disclosure provides a method for increasing the aggregation-resistance of a protein comprising an antibody heavy chain variable region (V_{H}), the method comprising modifying the V_{H} such that it comprises negatively charged amino acids at two or more positions selected from the group consisting of 28 and/or 31 and/or 32 and/or 33 and/or 35 according to the numbering system of Kabat, wherein the unmodified protein does not comprise the two or more negatively charged amino acids at positions 28 and/or 31 and/or 32 and/or 33 and/or 35 according to the numbering system of Kabat.

Additional sites of modification and/specific amino acid residues that can be substituted are described herein and are to be taken to apply mutatis mutandis to the present example.

In one example, the method comprises isolating a V_{H} from the protein, modifying the V_{H} according to a method of the disclosure and producing a protein comprising the V_{H}. For example, the method comprises isolating a V_{H} from an antibody, modifying the V_{H} according to a method of the disclosure and producing an antibody comprising the modified V_{H}.

In one example, a method of the disclosure additionally comprises affinity maturing the V_{H} or protein comprising same following modification according to the disclosure and/or deimmunizing the protein and/or humanizing the protein and/or chimerizing the protein.

In one example, a method of the disclosure does not involve inserting (as opposed to substituting) any additional amino acid residues into the V_{H}.

The methods described above are to be taken to apply mutatis mutandis to methods for increasing expression of a protein and/or for producing a protein capable of storage at high concentration with insignificant aggregation and/or for increasing recovery of a protein from a chromatography resin or for reducing the volume of solution required to recover a protein from a chromatography resin.

For example, the present disclosure provides a method for increasing the level of production of a soluble protein comprising an antibody V_{H}, the method comprising modifying the V_{H} by substituting an amino acid at position 28 and/or 31 and/or 33 and/or 35 according to the numbering system of Kabat with a negatively charged amino acid and producing the protein, wherein the level of soluble protein produced is increased compared to the level of production of protein lacking the negatively charged amino acids.

The present disclosure also provides a method for increasing the level of production of a soluble protein comprising an antibody V_{H}, the method comprising modifying the V_{H} by substituting two or more amino acids at position 28 and/or 31 and/or 32 and/or 33 and/or 35 according to the numbering system of Kabat with a negatively charged amino acid and contacting the protein with a chromatography resin, wherein the level of soluble protein produced is increased compared to the level of production of protein lacking the negatively charged amino acids.

The present disclosure also provides a method for increasing the level of recovery of a protein comprising an antibody heavy V_{H} from a chromatography resin or for reducing volume of solution required to recover the protein from a chromatography resin, the method comprising modifying the V_{H} by substituting an amino acid at position 28, 31, 33 and/or 35 according to the numbering system of Kabat with a negatively charged amino acid and contacting the protein with a chromatography resin, wherein the level of recovery of the protein recovered from a chromatography resin is increased or the volume of solution required to recover the protein from a chromatography resin is reduced compared to a protein lacking the negatively charged amino acids.

The present disclosure also provides a method for increasing the level of recovery of a protein comprising an antibody V_{H} from a chromatography resin or for reducing volume of solution required to recover the protein from a chromatography resin, the method comprising modifying the V_{H} by substituting two or more amino acids at position 28 and/or 31 and/or 32 and/or 33 and/or 35 according to the numbering system of Kabat with a negatively charged amino acid and contacting the protein with a chromatography resin, wherein the level of recovery of the protein recovered from a chromatography resin is increased or the volume of solution required to recover the protein from a chromatography resin is reduced compared to a protein lacking the negatively charged amino acids.

The present disclosure also provides for use of a protein of the present disclosure or a composition of the disclosure in medicine.

The present disclosure also provides a method of treating or preventing a condition in a subject, the method comprising administering a protein or composition of the disclosure to a subject in need thereof. In one example, the subject suffers from a cancer and/or an inflammatory disease and/or an autoimmune disease and/or a neurological condition.

The present disclosure also provides for use of a protein of the present disclosure in the manufacture of a medicament for the treatment or prevention of a condition.

The present disclosure also provides a method for delivering a compound to a cell, the method comprising contacting the cell with a protein or composition of the disclosure.

The present disclosure also provides a method for diagnosing or prognosing a condition in a subject, the method comprising contacting a sample from the subject with a protein or composition of the disclosure such that the protein binds to an antigen and form a complex and detecting the complex, wherein detection of the complex is diagnostic or prognostic of the condition in the subject. In one example, the method comprises determining the level of the complex, wherein an enhanced or reduced level of said complex is diagnostic or prognostic of the condition in the subject.

The present disclosure additionally provides a method for localising or detecting an antigen in a subject, said method comprising:
(i) administering to a subject a protein or composition of the disclosure such that the protein to binds to an antigen, wherein the protein is conjugated to a detectable label; and
(ii) detecting or localising the detectable label in vivo.

Each example of the present disclosure shall be taken to apply mutatis mutandis to a protein comprising an antibody heavy chain variable region (V_{H}) comprising a negatively charged amino acid position 30 according to the numbering system of Kabat optionally in combination with another site described herein, the protein capable of specifically binding to an antigen other than hen egg lysozyme, beta galactosidase, alpha amylase, B5R or wherein:
(i) if the protein binds to human tumor necrosis factor α and comprises aspartic acid at positions 30 and 31 it comprises at least one additional negatively charged amino acid between positions 28 and 35; and
(ii) if the protein binds to human tumor necrosis factor α and comprises glutamic acid at position 30 and aspartic acid at position 32 it comprises at least one additional negatively charged amino acid between positions 28 and 35.

### BRIEF DESCRIPTION OF THE DRAWINGS

FIG. 1 is a diagrammatic representation showing an amino acid sequence alignment of HEL4 (SEQ ID NO: 1) and DP47 (SEQ ID NO: 2). Identical amino acids are marked with an asterisk. Positions of CDRs as referred to herein are also indicated.

FIG. 2 is a graphical representation showing the results of aggregation-resistance experiments on DP47/HEL4 CDR chimeras. Introduction of HEL4 CDR1 into DP47 renders the V_{H} domain aggregation-resistant, while introduction of CDR2 and CDR3 has reduced effect.

FIG. 3 is a graphical representation showing results of experiments to identify single amino acids in the CDR1 of the HEL4 V_{H} domain, and combinations thereof that confer aggregation-resistance. Negatively charged amino acids at CDR1 positions 31, 32 or 33 resulted in a considerable aggregation-resistance of the V_{H} domain, while other mutations had limited effect. A triple amino acid change (SYA31-33DED) at 31-33 substantially increased aggregation-resistance. Positioning of any substitutions is indicated on the X axis.

FIG. 4 is a graphical representation showing aggregation-resistance of scFv comprising a V_{H} domain having single negative charged amino acid changes, and combinations thereof. Negatively charged amino acids at CDR1 positions 31, 32 or 33 resulted in a considerable aggregation-resistance of the scFv domain, while other mutations had limited effect. A triple amino acid change (SYA31-33DED) at 31-33 substantially increased aggregation-resistance of the scFv. Positioning of any substitutions is indicated on the X axis.

FIG. 5A is a graphical representation showing that the majority of tested native (unselected) clones from the Garvan-IA V_{H} library, comprising CDR1 of HEL4 (and thus negatively charged amino acids at positions 31, 32 and 33) and in which diversity was introduced into CDR3 of HEL4, exhibit a considerable level of aggregation-resistance when subjected to the “Heat/Cool” assay exemplified herein.

FIG. 5B is a graphical representation showing that the majority of tested naïve (unselected) clones from the Garvan-IB V_{H} library, comprising CDR1 of HEL4 (and thus negatively charged amino acids at positions 31, 32 and 33) and in which diversity was introduced into CDR3 of HEL4, exhibit a considerable level of aggregation-resistance when subjected to the “Heat/Cool” assay exemplified herein.

FIG. 6 shows specificity of antigen-binding of clone G07 anti-hTNF domain antibody and clone G11 anti-mIL-21 domain antibody. ELISA was used to determine the ability of each clone to bind to a variety of antigens (as shown in the drawing). “hTNF”, human tumor necrosis factor α; “mTNF”, mouse tumor necrosis factor α; “hIL21”, human interleukin 21; “mIL21”, mouse interleukin 21; “beta gal”, beta galactosidase; “hPRLR”, human prolactin receptor.

FIG. 7 is a graphical representation showing aggregation-resistance of various mutants of DP47 comprising negatively charged amino acids at surface exposed residues between positions 26 to 40. Results presented show aggregation-resistance of DP47 single mutants displayed on phage after being subjected to the “Heat/Cool” assay exemplified herein. Sites of mutations are indicated on the X-axis.

FIG. 8 is a graphical representation showing aggregation-resistance of various mutants of DP47 comprising negatively charged amino acids in CDR2. Results presented show aggregation-resistance of DP47 single mutants displayed on phage after being subjected to the “Heat/Cool” assay exemplified herein. Sites of mutations are indicated on the X-axis.

FIG. 9 is a graphical representation showing aggregation-resistance of various mutants of DP47 comprising multiple negatively charged amino acids in CDR1. Results presented show aggregation-resistance of DP47 single mutants displayed on phage after being subjected to the “Heat/Cool” assay exemplified herein. Sites of mutations are indicated on the X-axis.

FIG. 10 is a graphical representation showing aggregation-resistance of various mutants of DP47 comprising negatively charged amino acids at position 28 and/or 35 according to the numbering system of Kabat, optionally combined with negatively charged amino acids in CDR1. Results presented show aggregation-resistance of DP47 single mutants displayed on phage after being subjected to the “Heat/Cool” assay exemplified herein. Sites of mutations are indicated on the X-axis.

FIG. 11 is a graphical representation showing levels of expression of soluble DP47 mutants comprising single or multiple negatively charged amino acids in CDR1. Results presented show protein levels (mg per litre of culture) as determined using a protein A enzyme-linked immunosorbent assay (ELISA). Sites of mutations are indicated on the X-axis.

FIG. 12 is a graphical representation showing the percentage recovery of V_{H} domains following size exclusion chromatography. Various mutants of DP47 were heated 80° C. for 10 mins followed by cooling at 4° C. for 10 mins or not treated then exposed to size exclusion chromatography. Results are presented as the area under the curve of the heated sample, expressed as percentage of the area under the curve unheated sample. Sites of mutations are indicated on the X-axis.

FIGS. 13A and 13B are a series of graphical representation showing results of circular dichroism (CD) analysis of thermal unfolding of DP47 mutants. The aggregation-resistance of each sample was tested by heating samples to 80° C. and cooling the heated protein from 80° C. to 4° C. at 1° C./min. The identity of the V_{H} domain tested is indicated.

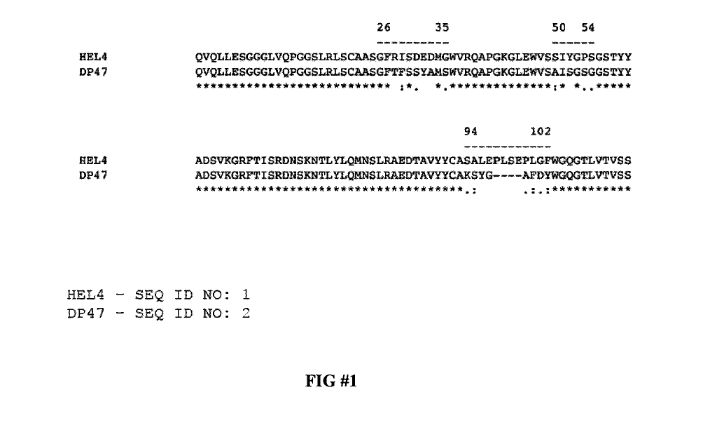

### KEY TO THE SEQUENCE LISTING

SEQ ID NO: 1—Amino acid sequence of HEL4 V_{H}.

SEQ ID NO: 2—Amino acid sequence of DP47 VH.

SEQ ID NO: 3—Amino acid sequence of VL region.

SEQ ID NO: 4—Amino acid sequence of linker sequence between VH and VL regions.

SEQ ID NO: 5—Amino acid sequence of VHhTNF_G07 (anti-hTNF VH).

SEQ ID NO: 6—Amino acid sequence of VHmIL21_G11 (anti-mIL-21 VH).

SEQ ID NO: 7—Amino acid sequence of VHPRLR_C02 (anti-hPRLR VH).

SEQ ID NO: 8—Amino acid sequence of VHHEL_H04 (anti-HEL VH).

SEQ ID NO: 9—Amino acid sequence of VHHEL_H08 (anti-HEL VH).

SEQ ID NO: 10—Amino acid sequence of VH region of adalimumab (sold as Humira®).

SEQ ID NO: 11—Amino acid sequence of VH region of rituximab (sold as Rituxan® or Mabthera®).

SEQ ID NO: 12—Amino acid sequence of VH region of trastuzumab (sold as Herceptin®).

SEQ ID NO: 13—Amino acid sequence of VH region of bevacizumab (sold as Avastin®).

SEQ ID NO: 14—Nucleotide sequence encoding HEL4 VH.

SEQ ID NO: 15—Nucleotide sequence encoding DP47 VH region.

SEQ ID NO: 16—Nucleotide sequence of oligonucleotide for amplifying VH.

SEQ ID NO: 17—Nucleotide sequence of oligonucleotide for amplifying VH.

### DETAILED DESCRIPTION OF PREFERRED EMBODIMENTS

### General

This specification contains nucleotide and amino acid sequence information prepared using PatentIn Version 3.5, presented herein after the claims.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any embodiment herein shall be taken to apply mutatis mutandis to any other embodiment unless specifically stated otherwise.

Any embodiment or example herein directed a protein comprising a V_{H} of an antibody or use thereof shall be taken to apply mutatis mutandis to a protein comprising a V_{H} of an immunoglobulin or use thereof.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat (1987 and/or 1991), Bork et al (1994) and/or Chothia and Lesk (1987 and 1989) or Al-Lazikani et al (1997).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### SELECTED DEFINITIONS

As used herein, the term “aggregation” means an association between or binding of proteins which is not reversible without treating the proteins with an agent that refolds the proteins into a native or unaggregated state. Such aggregation can lead to loss of function, loss of native fold, and/or gain of cytotoxicity or immunogenicity. This definition includes both detrimental and non-functional protein assemblies formed in vivo, and non-functional protein assemblies formed in vitro in biomedical research and biotechnology. It does not, however, include isoelectric or “salting out” precipitates, where the constituting proteins immediately return to their soluble native form upon transfer to native-like buffer conditions.

By “aggregation-resistant” is meant that following exposure to a condition that denatures a protein or a domain thereof (e.g., heat), a protein of the present disclosure is capable of refolding and binding to a binding partner in a conformation specific manner, for example, the protein is capable of refolding into a conformation that permits specific binding to an antigen and/or a superantigen, for example, Protein A. Preferably, following partial or complete denaturation (or unfolding) the protein is capable of refolding into a conformation that permits specific binding to the antigen or superantigen. Preferred proteins do not significantly aggregate following exposure to a condition that generally denatures a protein or a domain thereof (e.g. heat). For example, more than about 10% or 20% or 30% or 40% or 50% or 60% or 70% or 80% or 90% or 95% of the protein of the present disclosure in a composition comprising a plurality of said proteins do not aggregate following exposure to heat, e.g., 60° C. or 70° C. or 80° C. Accordingly, a preferred protein may also be considered heat refoldable.

As used herein, the term “antibody” shall be taken to mean a protein that comprises a variable region made up of a plurality of polypeptide chains, e.g., a light chain variable region (V_{L}) and a heavy chain variable region (V_{H}). An antibody also generally comprises constant domains, which can be arranged into a constant region or constant fragment or fragment crystallisable (Fc). Antibodies can bind specifically to one or a few closely related antigens. Generally, antibodies comprise a four-chain structure as their basic unit. Full-length antibodies comprise two heavy chains (approximately 50-70 kD) covalently linked and two light chains (approximately 23 kD each). A light chain generally comprises a variable region and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are approximately 110 amino acids in length) and one or more constant domains at the C-terminus. The constant domain of the light chain (C_{L} which is approximately 110 amino acids in length) is aligned with and disulfide bonded to the first constant domain of the heavy chain (C_{H} which is approximately 330-440 amino acids in length). The light chain variable region is aligned with the variable region of the heavy chain. The antibody heavy chain can comprise 2 or more additional C_{H} domains (such as, C_{H}2, C_{H}3 and the like) and can comprise a hinge region can be identified between the C_{H}1 and Cm constant domains. Antibodies can be of any type (e.g., IgG, IgE, IgM, IgD, IgA, and IgY), class (e.g., IgG₁, IgG₂, IgG₃, IgG₄, IgA₁ and IgA₂) or subclass. Preferably, the antibody is IgG, such as IgG₃. Preferably, the antibody is a murine (mouse or rat) antibody or a primate (preferably human) antibody. The term “antibody” also encompasses humanized antibodies, primatized antibodies, deimmunized antibodies, human antibodies and chimeric antibodies. This term does not encompass antibody-like molecules such as T cell receptors, such molecules are encompassed by the term “immunoglobulin”.

As used herein, “variable region” refers to the portions of the light and heavy chains of an antibody or immunoglobulin as defined herein that includes amino acid sequences of CDRs; i.e., CDR1, CDR2, and CDR3, and FRs. V_{H} refers to the variable region of the heavy chain. V_{L} refers to the variable region of the light chain. According to the methods used in this disclosure, the amino acid positions assigned to CDRs and FRs are defined according to Kabat (1987 and 1991) or Chothia (1989) and numbered according to the Kabat numbering system. The skilled artisan will be readily able to use other numbering systems in the performance of this disclosure, e.g., the hypervariable loop numbering system of Clothia and Lesk (1987) and/or Chothia (1989) and/or Al-Lazikani et al (1997).

As used herein, the term “heavy chain variable region” or “V_{H}” shall be taken to mean a protein capable of binding to one or more antigens, preferably specifically binding to one or more antigens and at least comprising a CDR1. Preferably, the heavy chain comprises three or four FRs (e.g., FR1, FR2, FR3 and optionally FR4) together with three CDRs. In one example, a heavy chain comprises FRs and CDRs positioned as follows residues 1-25 or 1-30 (FR1), 26-35 or 31-35 (or 35b) (CDR1), 36-49 (FR2), 50-65 (CDR2), 66-94 (FR3), 95-102 (CDR3) and 103-113 (FR4), numbered according to the Kabat numbering system. In one example, the heavy chain is derived from an immunoglobulin comprising said heavy chain and a plurality of (preferably 3 or 4) constant domains or linked to a constant fragment (Fc).

As used herein, the term “light chain variable region” or “V_{L}” shall be taken to mean a protein capable of binding to one or more antigens, preferably specifically binding to one or more antigens and at least comprising a CDR1. Preferably, the light chain comprises three or four FRs (e.g., FR1, FR2, FR3 and optionally FR4) together with three CDRs. Preferably, a light chain comprises FRs and CDRs positioned as follows residues 1-23 (FR1), 24-34 (CDR1), 35-49 (FR2), 50-56 (CDR2), 57-88 (FR3), 89-97 (CDR3) and 98-107 (FR4), numbered according to the Kabat numbering system. In one example, the light chain is derived from an immunoglobulin comprising said light chain linked to one constant domain and/or not linked to a constant fragment (Fc).

In some examples of the disclosure the term “framework regions” will be understood to mean those variable region residues other than the CDR residues. Each variable region of a naturally-occurring antibody typically has four FRs identified as FR1, FR2, FR3 and FR4. If the CDRs are defined according to Kabat, exemplary light chain FR (LCFR) residues are positioned at about residues 1-23 (LCFR1), 35-49 (LCFR2), 57-88 (LCFR3), and 98-107 (LCFR4). Note that λLCFR1 does not comprise residue 10, which is included in κLCFR1. Exemplary heavy chain FR (HCFR) residues are positioned at about residues 1-25 or 1-30 (HCFR1), 36-49 (HCFR2), 66-94 (HCFR3), and 103-113 (HCFR4).

As used herein, the term “complementarity determining regions” (syn. CDRs; i.e., CDR1, CDR2, and CDR3 or hypervariable region) refers to the amino acid residues of an antibody variable region the presence of which are necessary for antigen binding. Each variable region typically has three CDR regions identified as CDR1, CDR2 and CDR3. Each complementarity determining region may comprise amino acid residues from a “complementarity determining region” as defined by Kabat (1987 or 1991 or 1992) or Chotia (1989). In one preferred example of the present disclosure, in a heavy chain variable region CDRH1 is between residues 26-35 (or 35b), CDRH2 is between residues 50-65 and CDRH3 is between residues 95-102 numbered according to the Kabat numbering system. In a light chain CDRL1 is between residues 24-34, CDRL2 is between residues 50-56 and CDRL3 is between residues 89-97 numbered according to the Kabat numbering system. These CDRs can also comprise numerous insertions, e.g., as described in Kabat (1987 and/or 1991 and/or 1992).

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore an Fv of the disclosure (as well as any protein of the present disclosure) may have multiple antigen binding sites which may or may not bind the same antigen. This term shall be understood to encompass fragments directly derived from an antibody as well as proteins corresponding to such a fragment produced using recombinant means. In some examples; the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, a domain antibody (e.g., a V_{H}) or any of the foregoing linked to a constant region or domain thereof, e.g., C_{H}2 or C_{H}3 domain. A “Fab fragment” consists of a monovalent antigen-binding fragment of an antibody, and can be produced by digestion of a whole immunoglobulin with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain. Two Fab′ fragments are obtained per antibody treated in this manner. A Fab′ fragment can also be produced by recombinant means. An “F(ab′)2 fragment” of an antibody consists of a dimer of two Fab′ fragments held together by two disulfide bonds, and is obtained by treating a whole antibody with the enzyme pepsin, without subsequent reduction. An “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the V_{L} and V_{H} are covalently linked by a suitable, flexible polypeptide linker. A detailed discussion of exemplary Fv containing proteins falling within the scope of this term is provided herein below.

As used herein, the term “antigen binding site” shall be taken to mean a structure formed by a protein that is capable of specifically binding to an antigen. The antigen binding site need not be a series of contiguous amino acids, or even amino acids in a single polypeptide chain. For example, in a Fv produced from two different polypeptide chains the antigen binding site is made up of a series of regions of a V_{L} and a V_{H} that interact with the antigen and that are generally, however not always in the one or more of the CDRs in each variable region.

A “constant domain” is a domain in an antibody, the sequence of which is highly similar in antibodies of the same type, e.g., IgG or IgM or IgE. A constant region of an antibody generally comprises a plurality of constant domains, e.g., the constant region of γ, α and δ heavy chains comprise three constant domains and the Fc of γ, α and δ heavy chains comprise two constant domains. A constant region of μ and ε heavy chains comprises four constant domains and the Fc region comprises two constant domains.

The term “fragment crystalizable” or “Fc” as used herein, refers to a portion of an antibody comprising at least one constant domain and which is generally (though not necessarily) glycosylated and which binds to one or more Fc receptors and/or components of the complement cascade (e.g., confers effector functions). The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Preferred heavy chain constant regions are gamma 1 (IgG1), gamma 2 (IgG2) and gamma 3 (IgG3).

By “numbering system of Kabat” is meant the system for numbering residues in a variable region of an immunoglobulin in a consistent manner with the system set out in Kabat (1987 and/or 1991 and/or 1992).

The term “protein” shall be taken to include a single polypeptide, i.e., a series of contiguous amino acids linked by peptide bonds or a series of polypeptides covalently or non-covalently linked to one another (i.e., a polypeptide complex). For example, the series of polypeptides can be covalently linked using a suitable chemical or a disulphide bond. Examples of non-covalent bonds include hydrogen bonds, ionic bonds, Van der Waals forces, and hydrophobic interactions. A non-covalent bond contemplated by the present disclosure is the interaction between a V_{H} and a V_{L}, e.g., in some forms of diabody or a triabody or a tetrabody or an antibody.

The term “polypeptide” will be understood to mean from the foregoing paragraph to mean a series of contiguous amino acids linked by peptide bonds.

As used herein, the term “antigen” shall be understood to mean any composition of matter against which an immunoglobulin response (e.g., an antibody response) can be raised. Exemplary antigens include proteins, peptides, polypeptides, carbohydrates, phosphate groups, phosphor-peptides or polypeptides, glyscosylated peptides or peptides, etc.

As used herein, the term “specifically binds” or “binds specifically” shall be taken to mean a protein of the present disclosure reacts or associates more frequently, more rapidly, with greater duration and/or with greater affinity with a particular antigen or antigens or cell expressing same than it does with alternative antigens or cells. For example, a protein that specifically binds to an antigen binds that antigen with greater affinity, avidity, more readily, and/or with greater duration than it binds to other antigens. It is also understood by reading this definition that, for example, a protein that specifically binds to a first antigen may or may not specifically bind to a second antigen. As such, “specific binding” does not necessarily require exclusive binding or non-detectable binding of another antigen, this is meant by the term “selective binding”. Generally, but not necessarily, reference to binding means specific binding, and each term shall be understood to provide explicit support for the other term.

As used herein, the term “modified” in the context of a V_{H} means that the sequence of the V_{H} is changed compared to a parent (or unmodified) V_{H}. For example, a V_{H} comprising amino acids other than negatively charged amino acids at position 28 and/or 31 and/or 32 and/or 33 and/or 35 is modified to substitute one or more of those amino acids with a negatively charged amino acid. For example, a V_{H} is modified at position 28 and/or 31 and/or 32 and/or 33 and/or 35 to increase the number of negatively charged amino acids at these positions, e.g., to a total of 1 or 2 or 3 or 4 or 5 or more. In one exemplary form, the number of negatively charged amino acids at the recited positions is increased to at least two.

By “individually” is meant that the disclosure encompasses the recited residues or groups of residues separately, and that, notwithstanding that individual residue(s) or groups of residues may not be separately listed herein the accompanying claims may define such residue(s) or groups of residues separately and divisibly from each other.

By “collectively” is meant that the disclosure encompasses any number or combination of the recited residues or groups of residues, and that, notwithstanding that such numbers or combinations of residue(s) or groups of residues may not be specifically listed herein the accompanying claims may define such combinations or sub-combinations separately and divisibly from any other combination of residue(s) or groups of residues.

### Variable Region Containing Proteins

The present disclosure contemplates any protein that comprises an immunoglobulin heavy chain variable region that specifically or selectively binds to one or more antigens and that is modified as described herein according to any embodiment. The term “immunoglobulin” will be understood by the skilled artisan to include any protein of the immunoglobulin superfamily that comforms to the Kabat numbering system. Examples of immunoglobulin superfamily members include T cell receptors.

The present disclosure preferably contemplates any protein that comprises an antibody V_{H} that specifically or selectively binds to one or more antigens, e.g., by virtue of an antigen binding site and that is modified as described herein according to any embodiment.

### Antibody Variable Regions

As will be apparent to the skilled artisan based on the description herein, the proteins of the present disclosure can comprise one or more V_{H}s from an antibody modified to comprise a negatively charged amino acid at a position described herein. Such proteins include antibodies (e.g., an entire or full-length antibody). Such antibodies may be produced by first producing an antibody against an antigen of interest and modifying that antibody (e.g., using recombinant means) or by modifying a previously produced antibody. Alternatively, a protein comprising a V_{H} of the disclosure is produced, and that protein is then modified or used to produce an antibody.

Methods for producing antibodies are known in the art. For example, methods for producing monoclonal antibodies, such as the hybridoma technique, are described by Kohler and Milstein, (1975). In a hybridoma method, a mouse, hamster, or other appropriate host animal, is typically immunized with an immunogen or antigen or cell expressing same to elicit lymphocytes that produce or are capable of producing antibodies that will specifically bind to the immunogen or antigen. Lymphocytes or spleen cells from the immunized animals are then fused with an immortalized cell line using a suitable fusing agent, such as polyethylene glycol, to form a hybridoma cell (Goding, 1986). The resulting hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Other methods for producing antibodies are also contemplated by the present disclosure, e.g., using ABL-MYC technology described generically in detail in Largaespada (1990) or Weissinger et al. (1991).

Alternatively, the antibody, or sequence encoding same is generated from a previously produced cell expressing an antibody of interest, e.g., a hybridoma or transfectoma. Various sources of such hybridomas and/or transfectomas will be apparent to the skilled artisan and include, for example, American Type Culture Collection (ATCC) and/or European Collection of Cell Cultures (ECACC). Methods for isolating and/or modifying sequences encoding V_{H}s from antibodies will be apparent to the skilled artisan and/or described herein. Exemplary antibodies that can be modified according to the present disclosure include, but are not limited to, SYNAGIS® (Palivizumab; MedImmune) which is a humanized anti-respiratory syncytial virus (RSV) monoclonal antibody; HERCEPTIN® (Trastuzumab; Genentech) which is a humanized anti-HER2 monoclonal antibody; REMICADE® (infliximab; Centocor) which is a chimeric anti-TNFα monoclonal antibody; REOPRO® (abciximab; Centocor) which is an anti-glycoprotein Iib/IIIa receptor antibody; ZENAPAX® (daclizumab; Roche Pharmaceuticals) which is a humanized anti-CD25 monoclonal antibody; RITUXAN™/MABTHERA™ (Rituximab) which is a chimeric anti-CD20 IgG1 antibody (IDEC Pharm/Genentech, Roche); STIMULECT™ (basilimimab; Novartis), which is a chimeric anti-IL-2Rα antibody; ERBITUX (cetuximab; ImClone), which is a chimeric anti-EGFR antibody; MYLOTARG™ (gemtuzumab; Celltech/Wyeth), which is a humanized anti-CD33 antibody); Campath 1H/LDP-03 (Alemtuzumab; ILEX/Schering/Millenium) which is a humanized anti CD52 IgG1 antibody; XOLAIR™ (omalizumab; Tanox/Genentech/Novartis) a humanized anti-IgE Fc antibody; AVASTIN® (Bevacizumab; Genentech) humanized anti-VEGF antibody; RAPTIVA™ (Efalizumab; Genentech/Merck Serono) which is a humanized anti-CD11a antibody; LUCENTIS (Ranibizumab; Genentech/Novartis) which is a humanized anti-VEGF-A antibody; TYSABRI™ (Natalizumab; Biogen Idec/Élan Pharmaceuticals) which is a humanized anti-integrin-α4 antibody; SOLIRIS™ (eculizumab; Alexion Pharmaceuticals) which is a humanized anti-complement protein C5 antibody; VECTIBIX® (Panitumumab; Amgen), fully human anti-EGFR monoclonal antibody; or HUMIRA® (adalimumab; Abbott/MedImmune Cambridge) fully human anti-TNFα. Other antibodies and proteins comprising a VH of an antibody are known in the art and are not excluded.

Sequence of V_{H}s of known antibodies will be readily obtainable by a person skilled in the art. Exemplary sequences include, the V_{H} of adalimumab (SEQ ID NO: 10) or the V_{H} of rituximab (SEQ ID NO: 11) or the V_{H} of trastuzumab (SEQ ID NO: 12) or the V_{H} of bevacizumab (SEQ ID NO: 13). These sequences are readily modified according to the present disclosure.

Following antibody production and/or isolation of a sequence encoding same, the antibody or V_{H} thereof is modified to include negatively charged amino acids (e.g., aspartic acid or glutamic acid) in the requisite positions to confer aggregation-resistance, e.g., as described herein according to any embodiment. Generally, this comprises isolating the nucleic acid encoding the V_{H} or antibody and modifying the sequence thereof to include one or more codons encoding aspartic acid (i.e., GAA or GAG) or glutamic acid (i.e., GAT or GAC) at the requisite sites.

### Chimeric, Humanized and Human Antibodies

The proteins of the present disclosure may be derived from or may be a humanized antibody or a human antibody or V_{H} derived therefrom. The term “humanized antibody” shall be understood to refer to a chimeric molecule, generally prepared using recombinant techniques, having an antigen binding site derived from an antibody from a non-human species and the remaining antibody structure of the molecule based upon the structure and/or sequence of a human antibody. The antigen-binding site preferably comprises CDRs from the non-human antibody grafted onto appropriate FRs (i.e., the regions in a V_{H} other than CDRs) in the variable regions of a human antibody and the remaining regions from a human antibody. Antigen binding sites may be wild type or modified by one or more amino acid substitutions. In some instances, framework residues of the human antibody are replaced by corresponding non-human residues. Humanized antibodies may also comprise residues which are found neither in the recipient antibody nor in the imported CDR or framework sequences. In general, the humanized antibody will comprise substantially all of at least one, and typically two, variable regions, in which all or substantially all of the CDR regions correspond to those of a non-human antibody and all or substantially all of the FR regions are those of a human antibody consensus sequence. Methods for humanizing non-human antibodies are known in the art. Humanization can be essentially performed following the method of U.S. Pat. No. 5,225,539, U.S. Pat. No. 6,054,297 or U.S. Pat. No. 5,585,089. Other methods for humanizing an antibody are not excluded.

The term “human antibody” as used herein in connection with antibodies and binding proteins refers to antibodies having variable and, optionally, constant antibody regions derived from or corresponding to sequences found in humans, e.g. in the human germline or somatic cells. The “human” antibodies can include amino acid residues not encoded by human sequences, e.g. mutations introduced by random or site directed mutations in vitro (in particular mutations which involve conservative substitutions or mutations in a small number of residues of the antibody, e.g. in 1, 2, 3, 4 or 5 of the residues of the antibody, preferably e.g. in 1, 2, 3, 4 or 5 of the residues making up one or more of the CDRs of the antibody) and/or a negatively charged amino acid at a position described herein. Exemplary human antibodies or proteins comprise human framework regions (e.g., from the human germline) and random amino acids in the CDRs other than at the position(s) at which negatively charged amino acids are included. These “human antibodies” do not actually need to be produced by a human, rather, they can be produced using recombinant means and/or isolated from a transgenic animal (e.g., a mouse) comprising nucleic acid encoding human antibody constant and/or variable regions. Human antibodies or fragments thereof can be produced using various techniques known in the art, including phage display libraries (e.g., as described in Hoogenboom and Winter 1991; U.S. Pat. No. 5,885,793 and/or described below), or using transgenic animals expressing human immunoglobulin genes (e.g., as described in WO2002/066630; Lonberg et al. (1994) or Jakobovits et al. (2007)).

In one example, the protein of the present invention comprises a human V_{H}, i.e., at positions other than 28 and/or 31 and/or 32 and/or 33 and/or 35 according to the numbering system of Kabat. For example, the protein comprises completely human framework regions.

In one example, the protein does not comprise a humanized V_{H} or does not comprise a V_{H} derived from a humanized antibody. For example, the protein does not comprise murine amino acids in one or more framework regions. In one example, the protein does not comprise a V_{H} derived from humanized Fab4D5, e.g., as described in U.S. Pat. No. 6,407,213.

In one example a protein of the present disclosure is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass, as well as fragments of such antibodies, so long as they exhibit the desired biological activity (U.S. Pat. No. 4,816,567). Typically chimeric antibodies utilize rodent or rabbit variable regions and human constant regions, in order to produce an antibody with predominantly human domains. For example, a chimeric antibody comprises a variable region from a mouse antibody modified according to the present disclosure any embodiment fused to a human constant region. The production of such chimeric antibodies is known in the art, and may be achieved by standard means (as described, e.g., in U.S. Pat. No. 5,807,715; U.S. Pat. No. 4,816,567 and U.S. Pat. No. 4,816,397).

### V_{H} Containing Proteins

Single-Domain Antibodies

In some embodiments, a protein of the present disclosure is a single-domain antibody (which is used interchangeably with the term “domain antibody” or “dAb”). A single-domain antibody is a single polypeptide chain comprising all or a portion of the heavy chain variable domain of an antibody. In certain embodiments, a single-domain antibody is a human single-domain antibody (Domantis, Inc., Waltham, Mass.; see, e.g., U.S. Pat. No. 6,248,516; WO90/05144; WO2003/002609 and/or WO2004/058820). In one example, a single-domain antibody consists of all or a portion of the heavy chain variable domain of an antibody that is capable of specifically binding to an antigen and that is capable of modification according to the present disclosure. The present disclosure also encompasses a domain antibody fused to another molecule, e.g., another domain antibody or a Fc region.

Exemplary domain antibodies include a sequence set forth in any one of SEQ ID NOs: 5-9.

Diabodies, Triabodies, Tetrabodies

Exemplary proteins comprising a V_{H} are diabodies, triabodies, tetrabodies and higher order protein complexes such as those described in WO98/044001 and WO94/007921.

As used herein, the term “diabody” shall be taken to mean a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

As used herein, the term “triabody” shall be taken to mean a protein comprising three associated polypeptide chains, each polypeptide chain comprising the structure as set out above in respect of a diabody wherein the V_{H} of one polypeptide chain is associated with the V_{L} of another polypeptide chain to thereby form a trimeric protein (a triabody).

As used herein, the term “tetrabody” shall be taken to mean a protein comprising four associated polypeptide chains, each polypeptide chain comprising the structure set out above in respect of a diabody and wherein the V_{H} of one polypeptide chain is associated with the V_{L} of another polypeptide chain to thereby form a tetrameric protein (a tetrabody).

The skilled artisan will be aware of diabodies, triabodies and/or tetrabodies and methods for their production. The V_{H} and V_{L} can be positioned in any order, i.e., V_{L}-V_{H} or V_{H}-V_{L}. Generally, these proteins comprise a polypeptide chain in which a V_{H} and a V_{L} are linked directly or using a linker that is of insufficient length to permit the V_{H} and V_{L} to associate. Proteins comprising V_{H} and V_{L} associate to form diabodies, triabodies and/or tetrabodies depending on the length of the linker (if present) and/or the order of the V_{H} and V_{L} domains. Preferably, the linker comprises 12 or fewer amino acids. For example, in the case of polypeptide chains having the following structure arranged in N to C order V_{H}-X-V_{L}, wherein X is a linker, a linker having 3-12 residues generally results in formation of diabodies, a linker having 1 or 2 residues or where a linker is absent generally results in formation of triabodies. In the case of polypeptide chains having the following structure arranged in N to C order V_{L}-X-V_{H}, wherein X is a linker, a linker having 3-12 residues generally results in formation of diabodies, a linker having 1 or 2 residues generally results in formation of diabodies, triabodies and tetrabodies and a polypeptide lacking a linker generally forms triabodies or tetrabodies.

Exemplary publications describing diabodies, triabodies and/or tetrabodies include WO94/07921; WO98/44001; Holliger et al (1993); Hudson and Kortt (1999); Hollinger and Hudson (2005); and references cited therein.

Single Chain Fv (scFv)

The skilled artisan will be aware that scFvs comprise V_{H} and V_{L} regions in a single polypeptide chain. Preferably, the polypeptide chain further comprises a polypeptide linker between the V_{H} and V_{L} which enables the scFv to form the desired structure for antigen binding (i.e., for the V_{H} and V_{L} of the single polypeptide chain to associate with one another to form a Fv). This is distinct from a diabody or higher order multimer in which variable regions from different polypeptide chains associate or bind to one another. For example, the linker comprises in excess of 12 amino acid residues with (Gly₄Ser)₃ being one of the more favoured linkers for a scFv.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv (see, for example, Brinkmann et al., 1993).

Alternatively, or in addition, the present disclosure provides a dimeric scFv, i.e., a protein comprising two scFv molecules linked by a non-covalent or covalent linkage. Examples of such dimeric scFv include, for example, two scFvs linked to a leucine zipper domain (e.g., derived from Fos or Jun) whereby the leucine zipper domains associate to form the dimeric compound (see, for example, Kostelny 1992 or Kruif and Logtenberg, 1996). Alternatively, two scFvs are linked by a peptide linker of sufficient length to permit both scFvs to form and to bind to an antigen, e.g., as described in US20060263367.

Modified forms of scFv are also contemplated by the present disclosure, e.g., scFv comprising a linker modified to permit glycosylation, e.g., as described in U.S. Pat. No. 6,323,322.

The skilled artisan will be readily able to produce a scFv or modified form thereof comprising a suitable modified V_{H} according to the present disclosure based on the disclosure herein. For a review of scFv, see Plückthun (1994). Additional description of scFv is to be found in, for example, Bird et al., 1988.

Minibodies

The skilled artisan will be aware that a minibody comprises the V_{H} and V_{L} domains of an antibody fused to the C_{H}2 and/or C_{H}3 domain of an antibody. Optionally, the minibody comprises a hinge region between the V_{H} and a V_{L} and the C_{H}2 and/or C_{H}3 domains, sometimes this conformation is referred to as a Flex Minibody (Hu et al., 1996). A minibody does not comprise a C_{H}1 or a C_{L}. Preferably, the V_{H} and V_{L} domains are fused to the hinge region and the C_{H}3 domain of an antibody. Each of the regions may be derived from the same antibody. Alternatively, the V_{H} and V_{L} domains can be derived from one antibody and the hinge and C_{H}2/C_{H}3 from another, or the hinge and C_{H}2/C_{H}3 can also be derived from different antibodies. The present disclosure also contemplates a multispecific minibody comprising a V_{H} and V_{L} from one antibody and a V_{H} and a V_{L} from another antibody.

Exemplary minibodies and methods for their production are described, for example, in WO94/09817.

Other Variable Region Containing Proteins

U.S. Pat. No. 5,731,168 describes molecules in which the interface between a pair of Fv is engineered to maximize the percentage of heterodimers which are recovered from recombinant cell culture to thereby produce bi-specific proteins. The preferred interface comprises at least a part of a C_{H}3 domain. In this method, one or more small amino acid side chains from the interface of the first protein are replaced with larger side chains {e.g., tyrosine or tryptophan). Compensatory “cavities” of identical or similar size to the large side chain(s) are created on the interface of the second protein by replacing large amino acid side chains with smaller ones (e.g., alanine or threonine).

Bispecific proteins comprising variable regions include cross-linked or “heteroconjugate” proteins. For example, one of the proteins in the heteroconjugate can be coupled to avidin and the other to biotin. Such proteins have, for example, been proposed to target immune system cells to unwanted cells (U.S. Pat. No. 4,676,980). Heteroconjugate proteins comprising variable regions may be made using any convenient cross-linking methods. Suitable cross-linking agents are known in the art, and are disclosed in U.S. Pat. No. 4,676,980, along with a number of cross-linking techniques.

Bispecific proteins comprising variable regions can also be prepared using chemical linkage. Brennan (1985) describe a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific protein.

Additional variable region containing proteins include, for example, single chain Fab (e.g., Hust et al., 2007) or a Fab₃ (e.g., as described in EP19930302894).

### Constant Domain Fusions

The present disclosure encompasses a protein comprising a modified V_{H} of the disclosure and a constant region (e.g., Fc) or a domain thereof, e.g., C_{H}2 and/or C_{H}3 domain. For example, the present disclosure provides a minibody (as discussed above) or a domain antibody-Fc fusion or a scFv-Fc fusion or a diabody-Fc fusion or a triabody-Fc fusion or a tetrabody-Fc fusion or a domain antibody-C_{H}2 fusion, scFc-C_{H}2 fusion or a diabody-C_{H}2 fusion or a triabody-C_{H}2 fusion or a tetrabody-C_{H}2 fusion or a domain antibody-C_{H}3 fusion or a scFv-C_{H}3 fusion or a diabody-C_{H}3 fusion or a triabody-C_{H}3 fusion or a tetrabody-C_{H}3 fusion. Any of these proteins may comprise a linker, preferably an antibody hinge region, between the variable region and the constant region or constant domain. Preferably, such a Fc fusion protein has effector function.

As used herein, the term “C_{H}2 domain” includes the portion of a heavy chain antibody molecule that extends, e.g., from between about positions 231-340 according to the Kabat EU numbering system (as disclosed in Kabat 1991 or 1992). Two N-linked branched carbohydrate chains are generally interposed between the two CH₂ domains of an intact native IgG molecule. In one embodiment, a protein of the present disclosure comprises a C_{H}2 domain derived from an IgG1 molecule (e.g. a human IgG1 molecule). In another embodiment, a protein of the present disclosure comprises a C_{H}2 domain derived from an IgG4 molecule (e.g., a human IgG4 molecule).

As used herein, the term “C_{H}3 domain” includes the portion of a heavy chain antibody molecule that extends approximately 110 residues from N-terminus of the C_{H}2 domain, e.g., from about position 341-446b (Kabat EU numbering system). The C_{H}3 domain typically forms the C-terminal portion of an IgG antibody. In some antibodies, however, additional domains may extend from C_{H}3 domain to form the C-terminal portion of the molecule (e.g. the C_{H}4 domain in the μ chain of IgM and the e chain of IgE). In one example, a protein of the present disclosure comprises a C_{H}3 domain derived from an IgG1 molecule (e.g., a human IgG1 molecule). In another embodiment, a protein of the present disclosure comprises a C_{H}3 domain derived from an IgG4 molecule (e.g., a human IgG4 molecule).

Constant region sequences useful for producing the proteins of the present disclosure may be obtained from a number of different sources. In preferred examples, the constant region or portion thereof of the protein is derived from a human antibody. It is understood, however, that the constant region or portion thereof may be derived from an immunoglobulin or antibody of another mammalian species, including for example, a rodent (e.g. a mouse, rat, rabbit, guinea pig) or non-human primate (e.g. chimpanzee, macaque) species. Moreover, the constant region domain or portion thereof may be derived from any antibody class.

As used herein, the term “effector function” refers to the functional ability of the Fc region or portion thereof (e.g., C_{H}2 domain) to bind proteins and/or cells of the immune system and mediate various biological effects. Effector functions may be antigen-dependent or antigen-independent. “Antigen-dependent effector function” refers to an effector function which is normally induced following the binding of an antibody to an antigen. Typical antigen-dependent effector functions include the ability to bind a complement protein (e.g. C1q). For example, binding of the C1 component of complement to the Fc region can activate the classical complement system leading to the opsonisation and lysis of cell pathogens, a process referred to as complement-dependent cytotoxicity (CDC). The activation of complement also stimulates the inflammatory response and may also be involved in autoimmune hypersensitivity. Other antigen-dependent effector functions are mediated by the binding of antibodies, via their Fc region, to certain Fc receptors (“FcRs”) on cells. There are a number of Fc receptors which are specific for different classes of antibody, including IgG (gamma receptors, or IgλRs), IgE (epsilon receptors, or IgεRs), IgA (alpha receptors, or IgαRs) and IgM (μ receptors, or IgμRs). Binding of antibodies to Fc receptors on cell surfaces triggers a number of important and diverse biological responses including endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell-mediated cytotoxicity, or ADCC), release of inflammatory mediators, regulation of immune system cell activation, placental transfer and control of antibody production.

As used herein, the term “antigen-independent effector function” refers to an effector function which may be induced by an antibody, regardless of whether it has bound its corresponding antigen. Typical antigen-independent effector functions include cellular transport, circulating half-life and clearance rates of antibodies, and facilitation of purification. A structurally unique Fc receptor, the “neonatal Fc receptor” or “FcRn”, also known as the salvage receptor, plays a critical role in regulating half-life and cellular transport. Other Fc receptors purified from microbial cells (e.g. Staphylococcal Protein A or G) are capable of binding to the Fc region with high affinity and can be used to facilitate the purification of the Fc-containing protein.

Constant region domains can be cloned, e.g., using the polymerase chain reaction and primers which are selected to amplify the domain of interest. The cloning of antibody sequences is described in for example, in U.S. Pat. No. 5,658,570.

The protein of the present disclosure may comprise any number of constant regions/domains of different types.

The constant domains or portions thereof making up the constant region of an protein may be derived from different antibody molecules. For example, a protein may comprise a C_{H}2 domain or portion thereof derived from an IgG1 molecule and a C_{H}3 region or portion thereof derived from an IgG3 molecule.

In another example of the disclosure, the protein of the present disclosure comprises at least a region of an Fc sufficient to confer FcRn binding. For example, the portion of the Fc region that binds to FcRn comprises from about amino acids 282-438 of IgG1, according to Kabat EU numbering.

In one example, an altered protein of the present disclosure comprises a modified constant regions wherein or more constant region domains are partially or entirely deleted (“domain-deleted constant regions”). The present disclosure also encompasses modified Fc regions or parts there having altered, e.g., improved or reduced effector function. Many such modified Fc regions are known in the art and described, for example, in WO2005/035586, WO2005/063815 or WO2005/047327.

### Deimmunized Proteins

The present disclosure also contemplates a deimmunized protein. Deimmunized proteins have one or more epitopes, e.g., B cell epitopes or T cell epitopes removed (i.e., mutated) to thereby reduce the likelihood that a subject will raise an immune response against the protein. Methods for producing deimmunized proteins are known in the art and described, for example, in WO00/34317, WO2004/108158 and WO2004/064724. For example, the method comprises performing an in silico analysis to predict an epitope in a protein and mutating one or more residues in the predicted epitope to thereby reduce its immunogenicity. The protein is then analyzed, e.g., in silico or in vitro or in vivo to ensure that it retains its ability to bind to an antigen. Preferable an epitope that occurs within a CDR is not mutated unless the mutation is unlikely to reduce antigen binding. Methods for predicting epitopes are known in the art and described, for example, in Saha (2004).

Methods for introducing suitable mutations and expressing and assaying the resulting protein will be apparent to the skilled artisan based on the description herein.

### Libraries and Methods of Screening

The present disclosure also encompasses a library of proteins comprising a plurality of V_{H}s modified according to the present disclosure, e.g., the library comprises a plurality of proteins having with different binding characteristics

Examples of this disclosure include naïve libraries, immunized libraries or synthetic libraries. Naïve libraries are derived from B-lymphocytes of a suitable host which has not been challenged with any immunogen, nor which is exhibiting symptoms of infection or inflammation. Immunized libraries are made from a mixture of B-cells and plasma cells obtained from a suitably “immunized” host, i.e., a host that has been challenged with an immunogen. In one example, the mRNA from these cells is translated into cDNA using methods known in the art (e.g., oligo-dT primers and reverse transcriptase). In an alternative example, nucleic acids encoding antibodies from the host cells (mRNA or genomic DNA) are amplified by PCR with suitable primers. Primers for such antibody gene amplifications are known in the art (e.g., U.S. Pat. No. 6,096,551 and WO00/70023). In a further example, the mRNA from the host cells is synthesized into cDNA and these cDNAs are then amplified in a PCR reaction with antibody specific primers (e.g., U.S. Pat. No. 6,319,690). Alternatively, the repertoires may be cloned by conventional cDNA cloning technology (Sambrook and Russell, eds, Molecular Cloning: A Laboratory Manual, 3^{rd} Ed, vols. 1-3, Cold Spring Harbor Laboratory Press, 2001), without using PCR. The DNAs are modified to include negatively charged amino acid(s) at the requisite sites either during or following cloning.

In another example, a database of published antibody sequences of human origin is established where the antibody sequences are aligned to each other. The database is used to define subgroups of antibody sequences which show a high degree of similarity in both the sequence and the canonical fold of CDR loops (as determined by analysis of antibody structures). For each of the subgroups a consensus sequence is deduced which represents the members of this subgroup; the complete collection of consensus sequences represent therefore the complete structural repertoire of human antibodies.

These artificial genes are then constructed, e.g., by total gene synthesis or by the use of synthetic genetic subunits. These genetic subunits correspond to structural sub-elements at the polypeptide level. On the DNA level, these genetic subunits are defined by cleavage sites at the start and the end of each of the sub-elements, which are unique in the vector system. All genes which are members of the collection of consensus sequences are constructed such that they contain a similar pattern of corresponding genetic sub-sequences. For example, said polypeptides are or are derived from the HuCAL consensus genes: VκI, Vκ2, Vκ3, Vκ4, Vλ1, Vλ2, Vλ3, V_{H}1A, V_{H}1B, V_{H}2, V_{H}3, V_{H}4, V_{H}5, V_{H}6, Cκ, Cλ, C_{H}1 or any combination of said HuCAL consensus genes. This collection of DNA molecules can then be used to create “synthetic libraries” of antibodies, preferably Fv, disulphide-linked Fv, single-chain Fv (scFv), Fab fragments, or Fab′ fragments which may be used as sources of proteins that bind specifically to an antigen. U.S. Pat. No. 6,300,064 discloses methods for making synthetic libraries. Such synthetic libraries are modified to include a negatively charged amino acid according to the present disclosure. In another example, synthetic human antibodies are made by synthesis from defined V-gene elements. Winter (EP0368684) has provided a method for amplifying (e.g., by PCR), cloning, and expressing antibody variable region genes. Starting with these genes he was able to create libraries of functional antibody fragments by randomizing the CDR3 of the heavy and/or the light chain. This process is functionally equivalent to the natural process of VJ and VDJ recombination which occurs during the development of B-cells in the immune system. For example, repertoires of human germ line V_{H} gene segments can be rearranged in vitro by joining to synthetic “D-segments” of five random amino acid residues and a J-segment, to create a synthetic third complementarity determining region (CDR) of eight residues. U.S. Pat. No. 5,885,793 discloses methods of making such antibody libraries such as these. As will be apparent to the skilled artisan, a library comprising proteins of the present disclosure is produced such that the amplified V region comprises codons encoding a negatively charged amino acid at a position described herein.

The proteins according to the disclosure may be soluble secreted proteins or may be presented as a fusion protein on the surface of a cell, or particle (e.g., a phage or other virus, a ribosome or a spore).

Various display library formats are known in the art and reviewed, for example, in Levin and Weiss (2006). For example, the library is an in vitro display library (i.e., the proteins are displayed using in vitro display wherein the expressed domain is linked to the nucleic acid from which it was expressed such that said domain is presented in the absence of a host cell). Accordingly, libraries produced by in vitro display technologies are not limited by transformation or transfection efficiencies. Examples of methods of in vitro display include ribosome display, covalent display and mRNA display.

In one example, the in vitro display library is a ribosome display library. The skilled artisan will be aware that a ribosome display library directly links mRNA encoded by the expression library to the protein that it encodes. Means for producing a ribosome display library comprise placing nucleic acid encoding the protein comprising a V_{H} in operable connection with an appropriate promoter sequence and ribosome binding sequence. Preferred promoter sequences are the bacteriophage T3 and T7 promoters. Preferably, the nucleic acid is placed in operable connection with a spacer sequence and a modified terminator sequence with the terminator codon removed. As used in the present context, the term “spacer sequence” shall be understood to mean a series of nucleic acids that encode a peptide that is fused to the peptide. The spacer sequence is incorporated into the gene construct, as the peptide encoded by the spacer sequence remains within the ribosomal tunnel following translation, while allowing the protein comprising a V_{H} to freely fold and interact with another protein or a nucleic acid. A preferred spacer sequence is, for example, a nucleic acid that encodes amino acids 211-299 of gene III of filamentous phage M13 mp19.

The display library is transcribed and translated in vitro using methods known in the art and/or described for example, in Ausubel et al (1987) and Sambrook et al (2001). Examples of commercially available systems for in vitro transcription and translation include, for example, the TNT in vitro transcription and translation systems from Promega. Cooling the expression reactions on ice generally terminates translation. The ribosome complexes are stabilized against dissociation from the peptide and/or its encoding mRNA by the addition of reagents such as, for example, magnesium acetate or chloroamphenicol. Such in vitro display libraries are screened by a variety of methods, as described herein.

In another example, the display library of the present disclosure is a ribosome inactivation display library. In accordance with this example, a nucleic acid is operably linked to a nucleic acid encoding a first spacer sequence. It is preferred that this spacer sequence is a glycine/serine rich sequence that allows a protein comprising a V_{H} encoded therefrom to freely fold and interact with a target antigen. The first spacer sequence is linked to a nucleic acid that encodes a toxin that inactivates a ribosome. It is preferred that the toxin comprises the ricin A chain, which inactivates eukaryotic ribosomes and stalls the ribosome on the translation complex without release of the mRNA or the encoded peptide. The nucleic acid encoding the toxin is linked to another nucleic acid that encodes a second spacer sequence. The second spacer is an anchor to occupy the tunnel of the ribosome, and allow both the peptide and the toxin to correctly fold and become active. Examples of such spacer sequences are sequences derived from gene III of M13 bacteriophage. Ribosome inactivation display libraries are generally transcribed and translated in vitro, using a system such as the rabbit reticulocyte lysate system available from Promega. Upon translation of the mRNA encoding the toxin and correct folding of this protein, the ribosome is inactivated while still bound to both the encoded polypeptide and the mRNA from which it was translated.

In another example, the display library is a mRNA display library. In accordance with this embodiment, a nucleic acid is operably linked to a nucleic acid encoding a spacer sequence, such as a glycine/serine rich sequence that allows a protein comprising a V_{H} encoded by the expression library of the present disclosure to freely fold and interact with a target antigen. The nucleic acid encoding the spacer sequence is operably linked to a transcription terminator. mRNA display libraries are generally transcribed in vitro using methods known in the art, such as, for example, the HeLaScribe Nuclear Extract In Vitro Transcription System available from Promega. Encoded mRNA is subsequently covalently linked to a DNA oligonucleotide that is covalently linked to a molecule that binds to a ribosome, such as, for example, puromycin, using techniques known in the art and are described in, for example, Roberts and Szostak (1997). Preferably, the oligonucleotide is covalently linked to a psoralen moiety, whereby the oligonucleotide is photo-crosslinked to a mRNA encoded by the expression library of the present disclosure. The mRNA transcribed from the expression library is then translated using methods known in the art. When the ribosome reaches the junction of the mRNA and the oligonucleotide the ribosome stalls and the puromycin moiety enters the phosphotransferase site of the ribosome and thus covalently links the encoded polypeptide to the mRNA from which it was expressed.

In yet another example, the display library is a covalent display library. In accordance with this example, a nucleic acid encoding a protein comprising a V_{H} is operably linked to a second nucleic acid that encodes a protein that interacts with the DNA from which it was encoded. Examples of a protein that interacts with the DNA from which it interacts include, but are not limited to, the E. coli bacteriophage P2 viral A protein (P2A) and equivalent proteins isolated from phage 186, HP1 and PSP3. A covalent display gene construct is transcribed and translated in vitro, using a system such as the rabbit reticulocyte lysate system available from Promega. Upon translation of the fusion of the protein comprising a V_{H} and the P2A protein, the P2A protein nicks the nucleic acid to which it binds and forms a covalent bond therewith. Accordingly, a nucleic acid fragment is covalently linked to the peptide that it encodes.

In yet another example, the display library is a phage display library wherein the expressed proteins comprising a V_{H} are displayed on the surface of a bacteriophage, as described, for example, in U.S. Pat. No. 5,821,047; U.S. Pat. No. 6,248,516 and U.S. Pat. No. 6,190,908. The basic principle described relates to the fusion of a first nucleic acid comprising a sequence encoding a protein comprising a V_{H} to a second nucleic acid comprising a sequence encoding a phage coat protein, such as, for example a phage coat proteins selected from the group, M13 protein-3, M13 protein-7, or M13, protein-8. These sequences are then inserted into an appropriate vector, i.e., one that is able to replicate in bacterial cells. Suitable host cells, such as, for example E. coli, are then transformed with the recombinant vector. Said host cells are also infected with a helper phage particle encoding an unmodified form of the coat protein to which a nucleic acid fragment is operably linked. Transformed, infected host cells are cultured under conditions suitable for forming recombinant phagemid particles comprising more than one copy of the fusion protein on the surface of the particle. This system has been shown to be effective in the generation of virus particles such as, λ phage, T4 phage, M13 phage, T7 phage and baculovirus. Such phage display particles are then screened to identify a displayed domain having a conformation sufficient for binding to a target antigen.

Other viral display libraries include a retroviral display library wherein the expressed peptides or protein domains are displayed on the surface of a retroviral particle, e.g., as described in U.S. Pat. No. 6,297,004

The present disclosure also contemplates bacterial display libraries, e.g., as described in U.S. Pat. No. 5,516,637; yeast display libraries, e.g., as described in U.S. Pat. No. 6,423,538 or a mammalian display library, e.g., as described in Strenglin et al 1988.

Methods for screening display libraries are known in the art. In one example, a display library of the present disclosure is screened using affinity purification. Affinity purification techniques are known in the art and are described in, for example, Scopes (1994). Methods of affinity purification typically involve contacting the proteins comprising a V_{H} displayed by the library with a target antigen and/or a superantigen (e.g., Protein A) and, following washing, eluting those domains that remain bound to the antigen. The antigen is preferably bound to another molecule to allow for ease of purification, such as, for example, a molecule selected from the group consisting of protein G, Sepharose, agarose, biotin, glutathione S-transferase (GST), and FLAG epitope. Accordingly, the target protein or nucleic acid is isolated simply through centrifugation, or through binding to another molecule, e.g. streptavidin, or binding of a specific antibody, e.g. anti-FLAG antibodies, or anti-GST antibodies.

In another example, the display library of the present disclosure is expressed so as to allow identification of a bound peptide using FACS analysis. The screening of libraries using FACS analysis is described in US6,455,63. Preferably, an in vitro display library is screened by FACS sorting. In vitro display proteins are covalently linked to a particle or bead suitable for FACS sorting, such as, for example, glass, polymers such as for example polystyrene, latex or cross-linked dextrans such as Sepharose, cellulose, nylon, Teflon, amongst others. The displayed library bound to particles or beads is added to a antigen or superantigen that has been labeled with a detectable label, such as for example a fluorescent molecule, or a molecule which is detected by a second fluorescent molecule. The beads are then washed and subjected to sorting by FACS, which allows the beads with bound fluorescent antigen or superantigen, to be separated from the beads that have not bound to a fluorescent target protein or nucleic acid.

Alternatively the library is screened using a biosensor-based assay, such as, for example, Biacore sensor chip technology (Biacore AB, UK). The Biacore sensor chip is a glass surface coated with a thin layer of gold modified with carboxymethylated dextran, to which the target protein or nucleic acid is covalently attached. The libraries of the present disclosure are then exposed to the Biacore sensor chip comprising the antigen.

### Protein Production

### Mutagenesis

DNA encoding a protein comprising a variable region is isolated using standard methods in the art. For example, primers are designed to anneal to conserved regions within a variable region that flank the region of interest, and those primers are then used to amplify the intervening nucleic acid, e.g., by PCR. Suitable methods and/or primers are known in the art and/or described, for example, in Borrebaeck (ed), 1995 and/or Froyen et al., 1995. Suitable sources of template DNA for such amplification methods is derived from, for example, hybridomas, transfectomas and/or cells expressing proteins comprising a variable region, e.g., as described herein.

Following isolation, the DNA is modified to include codons encoding negatively charged amino acid at the requisite locations by any of a variety of methods known in the art. These methods include, but are not limited to, preparation by site-directed (or oligonucleotide-mediated) mutagenesis, PCR mutagenesis, and cassette mutagenesis of an earlier prepared DNA encoding the protein. Variants of recombinant proteins may be constructed also by restriction fragment manipulation or by overlap extension PCR with synthetic oligonucleotides. Mutagenic primers encode the negatively charged amino acids, for example include residues that make up a codon encoding a negatively charged amino acid, e.g., aspartic acid (i.e., GAA or GAG) or glutamic acid (i.e., GAT or GAC). Standard mutagenesis techniques can be employed to generate DNA encoding such mutant DNA. General guidance can be found in Sambrook et al 1989; and/or Ausubel et al 1993.

Site-directed mutagenesis is one method for preparing substitution variants, i.e. mutant proteins. This technique is known in the art (see for example, Carter et al 1985; or Ho et al 1989). Briefly, in carrying out site-directed mutagenesis of DNA, the starting DNA is altered by first hybridizing an oligonucleotide encoding the desired mutation (e.g., insertion of one or more negatively charged amino acid encoding codons) to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA. Site-directed mutagenesis may be carried out within the gene expressing the protein to be mutagenized in an expression plasmid and the resulting plasmid may be sequenced to confirm the introduction of the desired negatively charged amino acid replacement mutations. Site-directed protocols and formats include commercially available kits, e.g. QuikChange® Multi Site-Directed Mutagenesis Kit (Stratagene, La Jolla, Calif.).

PCR mutagenesis is also suitable for making amino acid sequence variants of the starting protein. See Higuchi, 1990; Ito et al 1991. Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al, 1985. The starting material is the plasmid (or other vector) comprising the starting protein DNA to be mutated. The codon(s) in the starting DNA to be mutated are identified. There must be a unique restriction endonuclease site on each side of the identified mutation site(s). If no such restriction sites exist, they may be generated using the above described oligonucleotide-mediated mutagenesis method to introduce them at appropriate locations in the starting DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence. Mutant DNA containing the encoded negatively charged amino acid replacements can be confirmed by DNA sequencing.

Single mutations are also generated by oligonucleotide directed mutagenesis using double stranded plasmid DNA as template by PCR based mutagenesis (Sambrook et al., 2001).

### Recombinant Expression

In the case of a recombinant protein, nucleic acid encoding same is preferably placed into expression vectors, which are then transfected into host cells, preferably cells that can produce a disulphide bridge or bond, such as E. coli cells, yeast cells, insect cells, or mammalian cells, such as simian COS cells, Chinese Hamster Ovary (CHO) cells, or myeloma cells that do not otherwise produce immunoglobulin protein, to obtain the synthesis of proteins in the recombinant host cells. Review articles on recombinant expression in bacteria of DNA encoding antibodies include Skerra et al, (1993) and Plückthun, (1992). Molecular cloning techniques to achieve these ends are known in the art and described, for example in Ausubel et al (1987) and Sambrook et al (2001). A wide variety of cloning and in vitro amplification methods are suitable for the construction of recombinant nucleic acids. Methods of producing recombinant antibodies are also known in the art. See U.S. Pat. No. 4,816,567.

Following isolation, the nucleic acid encoding a protein of the present disclosure is preferably inserted into an expression construct or replicable vector for further cloning (amplification of the DNA) or for expression in a cell-free system or in cells. Preferably, the nucleic acid is operably linked to a promoter,

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Preferred promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Cell free expression systems are also contemplated by the present disclosure. For example, a nucleic acid encoding a protein of the present disclosure is operably linked to a suitable promoter, e.g., a T7 promoter, and the resulting expression construct exposed to conditions sufficient for transcription and translation. Typical expression vectors for in vitro expression or cell-free expression have been described and include, but are not limited to the TNT T7 and TNT T3 systems (Promega), the pEXP1-DEST and pEXP2-DEST vectors (Invitrogen).

Many vectors for expression in cells are available. The vector components generally include, but are not limited to, one or more of the following: a signal sequence, a sequence encoding protein of the present disclosure (e.g., derived from the information provided herein), an enhancer element, a promoter, and a transcription termination sequence. The skilled artisan will be aware of suitable sequences for expression of a protein. For example, exemplary signal sequences include prokaryotic secretion signals (e.g., pelB, alkaline phospholipase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters include those active in prokaryotes (e.g., phoA promoter, β-lactamase and lactose promoter systems, alkaline phosphatase, a tryptophan (trp) promoter system, and hybrid promoters such as the tac promoter). These promoter are useful for expression in prokaryotes including eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces. Preferably, the host is E. coli. One preferred E. coli cloning host is E. coli 294 (ATCC 31,446), although other strains such as E. coli B, E. coli X 1776 (ATCC 31,537), and E. coli W3110 (ATCC 27,325), DH5α or DH10B are suitable.

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Typical promoters suitable for expression in insect cells include, but are not limited to, the OPEI2 promoter, the insect actin promoter isolated from Bombyx muri, the Drosophila sp. Dsh promoter (Marsh et al 2000) and the inducible metallothionein promoter. Preferred insect cells for expression of recombinant proteins include an insect cell selected from the group comprising, BT1-TN-5B1-4 cells, and Spodoptera frugiperda cells (e.g., sf19 cells, sf21 cells). Suitable insects for the expression of the nucleic acid fragments include but are not limited to Drosophila sp. The use of S. frugiperda is also contemplated.

Means for introducing the isolated nucleic acid molecule or a gene construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein of this disclosure may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

### Isolation of Proteins

A protein of the present disclosure is preferably isolated. By “isolated” is meant that the protein is substantially purified or is removed from its naturally-occurring environment, e.g., is in a heterologous environment. By “substantially purified” is meant the protein is substantially free of contaminating agents, e.g., at least about 70% or 75% or 80% or 85% or 90% or 95% or 96% or 97% or 98% or 99% free of contaminating agents.

Methods for purifying a protein of the present disclosure are known in the art and/or described herein. For example, the protein is contacted with an agent capable of binding thereto for a time and under conditions sufficient for binding to occur. Optionally, following washing to remove unbound protein, the protein of the present disclosure is isolated, e.g., eluted.

When using recombinant techniques, the protein of the present disclosure can be produced intracellularly, in the periplasmic space, or directly secreted into the medium. If the protein is produced intracellularly, as a first step, the particulate debris, either host cells or lysed fragments, is removed, for example, by centrifugation or ultrafiltration. Carter et al. (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the protein is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein prepared from the cells can be purified using, for example, hydroxyl apatite chromatography, gel electrophoresis, dialysis, and affinity chromatography, with affinity chromatography being the preferred purification technique. The suitability of protein A as an affinity ligand depends on the species and isotype of any antibody Fc domain that is present in the protein (if present at all). Protein A can be used to purify antibodies that are based on human γ1, γ2, or γ4 heavy chains (Lindmark et al. 1983). Protein G is recommended for all mouse isotypes and for human γ3 (Guss et al. 1986). Otherwise affinity purification can be performed using the antigen or epitopic determinant to which a variable region in a protein of the present disclosure binds or was raised. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the protein to be recovered.

The skilled artisan will also be aware that a protein of the present disclosure can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. Preferably, the tag is a hexa-his tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilised on a solid or semi-solid support, washing the sample to remove unbound protein, and subsequently eluting the bound protein. Alternatively, or in addition a ligand or antibody that binds to a tag is used in an affinity purification method.

Following any preliminary purification step(s), the mixture comprising the protein of the present disclosure and contaminants may be subjected to low pH hydrophobic interaction chromatography.

### Protein Synthesis

A protein of the present disclosure is readily synthesized from its determined amino acid sequence using standard techniques, e.g., using BOC or FMOC chemistry. Synthetic peptides are prepared using known techniques of solid phase, liquid phase, or peptide condensation, or any combination thereof, and can include natural and/or unnatural amino acids. Amino acids used for peptide synthesis may be standard Boc (Nα-amino protected Nα-t-butyloxycarbonyl) amino acid resin with the deprotecting, neutralization, coupling and wash protocols of the original solid phase procedure of Merrifield, 1963, or the base-labile Nα-amino protected 9-fluorenylmethoxycarbonyl (Fmoc) amino acids described by Carpino and Han, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

### Methods of Evaluating Protein Aggregation-Resistance

The aggregation-resistance of the proteins or compositions of the disclosure can be analyzed using methods known in the art. Aggregation-resistance parameters acceptable to those in the art may be employed. Exemplary parameters are described in more detail below. In exemplary embodiments, thermal refoldability is evaluated. In some examples, the expression levels (e.g., as measured by % yield) of the protein of the present disclosure are evaluated. In other examples, the aggregation levels of the proteins of the present disclosure are evaluated. In certain examples, the aggregation-resistance of a protein or composition of an disclosure is compared with that of a suitable control.

The aggregation-resistance of a protein of the present disclosure may be analyzed using a number of non-limiting biophysical or biochemical techniques known in the art. An example of such a technique is analytical spectroscopy, such as Circular Dichroism (CD) spectroscopy. CD spectroscopy measures the optical activity of a protein as a function of increasing temperature. Circular dichroism (CD) spectroscopy measures differences in the absorption of left-handed polarized light versus right-handed polarized light which arise due to structural asymmetry. A disordered or unfolded structure results in a CD spectrum very different from that of an ordered or folded structure. The CD spectrum reflects the sensitivity of the proteins to the denaturing effects of increasing temperature and is therefore indicative of a protein's aggregation-resistance (see van Mierlo and Steemsma, 2000).

Another exemplary analytical spectroscopy method for measuring aggregation-resistance is Fluorescence Emission Spectroscopy (see van Mierlo and Steemsma, supra). Yet another exemplary analytical spectroscopy method for measuring aggregation-resistance is Nuclear Magnetic Resonance (NMR) spectroscopy (see, e.g. van Mierlo and Steemsma, supra).

In other embodiments, the aggregation-resistance of a composition or protein of the present disclosure is measured biochemically. An exemplary biochemical method for assessing aggregation-resistance is a thermal challenge assay. In a “thermal challenge assay”, a protein of the present disclosure is subjected to a range of elevated temperatures for a set period of time. For example, a test protein or is subject to an range of increasing temperatures. The activity of the protein is then assayed by a relevant biochemical assay. For example, the binding activity of the binding protein may be determined by a functional or quantitative ELISA. Another method for determining binding affinity employs surface plasmon resonance. Surface plasmon resonance is an optical phenomenon that allows for the analysis of real-time bispecific interactions by detection of alterations in protein concentrations within a biosensor matrix, for example using the BIAcore system (Pharmacia Biosensor AB, Uppsala, Sweden and Piscataway, N.J.).

In other examples, the aggregation-resistance of a composition or protein of the present disclosure is determined by measuring its propensity to aggregate. Aggregation can be measured by a number of non-limiting biochemical or biophysical techniques. For example, the aggregation of a composition or protein of the present disclosure may be evaluated using chromatography, e.g. Size-Exclusion Chromatograpy (SEC). SEC separates molecules on the basis of size. A column is filled with semi-solid beads of a polymeric gel that will admit ions and small molecules into their interior but not large ones. When a protein or composition is applied to the top of the column, the compact folded proteins (i.e., non-aggregated proteins) are distributed through a larger volume of solvent than is available to the large protein aggregates. Consequently, the large aggregates move more rapidly through the column, and in this way the mixture can be separated or fractionated into its components. Each fraction can be separately quantified (e.g. by light scattering) as it elutes from the gel. Accordingly, the percentage aggregation of a protein or composition of the disclosure can be determined by comparing the concentration of a fraction with the total concentration of protein applied to the gel. Aggregation-resistant compositions elute from the column as essentially a single fraction and appear as essentially a single peak in the elution profile or chromatogram.

In other examples, the aggregation-resistance of a composition of the disclosure is evaluated by measuring the amount of protein that is recovered (herein the “% yield”) following expression (e.g. recombinant expression) of the protein. For example, the % yield can be measured by determining milligrams of protein recovered for every ml of host culture media (e.g., mg/ml of protein). In a preferred example, the % yield is evaluated following expression in a mammalian host cell (e.g. a CHO cell).

In yet another example, the aggregation-resistance of a composition of the disclosure is evaluated by monitoring the loss of protein at a range of temperatures (e.g. from about 25° C. to about 80° C.) following storage for a defined time period. The amount or concentration of recovered protein can be determined using any protein quantification method known in the art, and compared with the initial concentration of protein. Exemplary protein quantification methods include SDS-PAGE analysis or the Bradford assay.

In yet other examples, the aggregation-resistance of a protein of the present disclosure may be assessed by quantifying the binding of a labeled compound to denatured or unfolded portions of a binding molecule. Such molecules are preferably hydrophobic, as they preferably bind or interact with large hydrophobic patches of amino acids that are normally buried in the interior of the native protein, but which are exposed in a denatured or unfolded binding molecule. An exemplary labeled compound is the hydrophobic fluorescent dye, 1-anilino-8-naphthaline sulfonate (ANS).

Other examples, involve detecting binding of a protein that only binds to a correctly folded variable domain (e.g., Protein A binds to correctly folded IgG3 V_{H})

### Conjugates

The present disclosure also provides proteins of the present disclosure conjugated to another compound, e.g., a conjugate (immunoconjugate) comprising an protein of the present disclosure conjugated to a distinct moiety, e.g., a therapeutic agent which is directly or indirectly bound to the protein. Examples of other moieties include, but are not limited to, an enzyme, a fluorophophore, a cytotoxin, a radioisotope (e.g., iodine-131, yttrium-90 or indium-111), an immunomodulatory agent, an anti-angiogenic agent, an anti-neovascularization and/or other vascularization agent, a toxin, an anti-proliferative agent, a pro-apoptotic agent, a chemotherapeutic agent and a therapeutic nucleic acid.

A cytotoxin includes any agent that is detrimental to (e.g., kills) cells. For a description of these classes of drugs which are known in the art, and their mechanisms of action, see Goodman et al. (1990). Additional techniques relevant to the preparation of antibody immunotoxins are provided in for instance U.S. Pat. No. 5,194,594. Exemplary toxins include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO93/21232.

Suitable therapeutic agents for forming immunoconjugates of the present disclosure include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)).

A variety of radionuclides are available for the production of radioconjugated antibodies. Examples include, but are not limited to, ²¹²Bi, ¹³¹I, ⁹⁰Y, and ¹⁸⁶Re.

In another embodiment, the protein may be conjugated to a “receptor” (such as streptavidin) for utilization in pretargeting wherein the protein-receptor conjugate is administered to the patient, followed by removal of unbound conjugate from the circulation using a clearing agent and then administration of a “ligand” (e.g., avidin) that is conjugated to a therapeutic agent (e.g., a radionucleotide).

The proteins of the present disclosure can be further modified to contain additional nonproteinaceous moieties that are known in the art and readily available. Preferably, the moieties suitable for derivatization of the protein are water soluble polymers. Non-limiting examples of water soluble polymers include, but are not limited to, polyethylene glycol (PEG), copolymers of ethylene glycol/propylene glycol, carboxymethylcellulose, dextran or polyvinyl alcohol.

Various methods are known in the art for conjugating a compound to a protein residue are known in the art and will be apparent to the skilled artisan.

### Uses

The proteins of the present disclosure are useful in a variety of applications, including research, diagnostic/prognostic, industrial and therapeutic applications. Depending on the antigen to which the protein binds it may be useful for delivering a compound to a cell, e.g., to kill the cell or prevent growth and/or for imaging and/or for in vitro assays. In one example, the protein is useful for both imaging and delivering a cytotoxic agent to a cell, i.e., it is conjugated to a detectable label and a cytotoxic agent or a composition comprises a mixture of proteins some of which are conjugated to a cytotoxic agent and some of which are conjugated to a detectable label.

The proteins described herein can also act as antagonists to inhibit (which can be reducing or preventing) (a) binding (e.g., of a ligand, an inhibitor) to a receptor, (b) a receptor signalling function, and/or (c) a stimulatory function. Proteins which act as antagonists of receptor function can block ligand binding directly or indirectly (e.g., by causing a conformational change).

A protein of the present disclosure may also be an agonist of a receptor, e.g., (a) enhancing or inducing binding (e.g., of a ligand) to a receptor, (b) enhancing or inducing receptor signalling function, and/or (c) providing a stimulatory function.

### Antigens

The present disclosure contemplates a protein comprising at least one V_{H} modified according to the present disclosure capable of specifically binding to any antigen(s) other than those specifically excluded in any embodiment, example or claim herein, i.e., an example of the disclosure is generic as opposed to requiring a specific antigen.

In one example, the protein of the present disclosure does not bind to a protein from a microorganism and/or from an avian.

In one example, the protein does not bind to lysozyme (e.g., hen egg lysozyme) and/or beta-galactosidase and/or amylase (e.g., alpha amylase) and/or anhydrase (e.g., carbonic anhydrase) and or B5R (e.g., from Vaccinia). In one example the protein does not bind to human albumin. In one example, the protein does not binds to human VEGF.

Preferred proteins bind specifically to a human protein or are derived from antibodies raised against a human protein.

Examples of the present disclosure contemplate a protein that specifically binds to an antigen associated with a disease or disorder (i.e., a condition) e.g., associated with or expressed by a cancer or cancerous/transformed cell and/or associated with an autoimmune disease and/or associated with an inflammatory disease or condition and/or associated with a neurodegenerative disease and/or associated with an immune-deficiency disorder.

Exemplary antigens against which a protein of the present disclosure can be produced include BMPR1B (bone morphogenetic protein receptor-type IB; WO2004063362); E16 (LAT1, SLC7A5, WO2004048938); STEAP1 (six transmembrane epithelial antigen of prostate, WO2004065577); CA125 (MUC16, WO2004045553); MPF (MSLN, SMR, megakaryocyte potentiating factor, mesothelin, WO2003101283); Napi3b (WO2004022778); Sema 5b (WO2004000997); PSCA (US2003129192); ETBR (WO2004045516); MSG783 (WO2003104275); STEAP2 (WO2003087306); TrpM4 (US2003143557); CRIPTO (US2003224411); CD21 (WO2004045520); CD79b (WO2004016225); SPAP1B (WO2004016225); HER2 (WO2004048938); NCA (WO2004063709); MDP (WO2003016475); IL-20Rα (EP1394274); Brevican (US2003186372); EphB2R (WO2003042661); ASLG659 (US20040101899); PSCA (WO2004022709); GEDA (WO2003054152); BAFF-R (WO2004058309); CD22 (WO2003072036); CD79a (WO2003088808); CXCR5 (WO2004040000); HLA-DOB (WO9958658); P2X5 (WO2004047749); CD72 (WO2004042346); LY64 (US2002193567); FcRH1 (WO2003077836); IRTA2 (WO2003077836); TENB2 (WO2004074320); CD20 (WO94/11026); VEGF-A (Presta et al., 1997); p53; EGFR; progesterone receptor; cathepsin D; Bcl-2; E cadherin; CEA; Lewis X; Ki67; PCNA; CD3; CD4; CD5; CD7; CD11c; CD11d; c-Myc; tau; PrPSC; TNFα; sonic hedgehog; hepatocyte growth factor; hepatocyte growth factor receptor; EPHA2; prolactin receptor; prolactin; IL-2; TNF-Receptor; IL-21; IL-21 Receptor; CXCR7; FGFR2; FGF2 or Aβ.

In another example, a protein of the present disclosure binds to a soluble protein, preferably a soluble protein that is secreted in vivo. Exemplary soluble proteins include cytokines. The term “cytokine” is a generic term for proteins or peptides released by one cell population which act on another cell as intercellular mediators. Examples of cytokines include lymphokines, monokines, growth factors and traditional polypeptide hormones. Included among the cytokines are growth hormones such as human growth hormone, N-methionyl human growth hormone, and bovine growth hormone; parathyroid hormone, thyroxine, insulin, proinsulin, relaxin, prorelaxin, glycoprotein hormones such as follicle stimulating hormone (FSH), thyroid stimulating hormone (TSH) and luteinizing hormone (LH), hepatic growth factor; prostaglandin, fibroblast growth factor, prolactin, placental lactogen, OB protein, tumor necrosis factor-α and -β; mullerian-inhibiting substance, gonadotropin-associated peptide, inhibin, activin, vascular endothelial growth factor, integrin, thrombopoietin (TPO), nerve growth factors such as NGF-B, platelet-growth factor, transforming growth factors (TGFs) such as TGF-α and TGF-β, insulin-like growth factor-I or -II, erythropoietin (EPO), osteoinductive factors, interferons such as interferon-α, -β, or -γ; colony stimulating factors (CSFs) such as macrophage-CSF (M-CSF), granulocyte-macrophage-CSF (GM-CSF); and granulocyte-CSF (G-CSF), interleukins (Ils) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, IL-21 and LIF. Preferred cytokines are selected from the group consisting of Interleukin 2, 13 or 21, TNF alpha, TGF beta, BAFF and GM-CSF.

In another example, a soluble protein is a chemokine. Chemokines generally act as chemoattractants to recruit immune effector cells to the site of chemokine expression. Chemokines include, but are not limited to, RANTES, MCAF, M1P1-alpha or MIP1-Beta. The skilled artisan will recognize that certain cytokines are also known to have chemoattractant effects and could also be classified under the term chemokines. A preferred chemokine is RANTES.

In another example, a soluble protein is a peptide hormone. Exemplary peptide hormones include insulin, NPY, PYY, glucagon and prolactin.

In a further example, a soluble protein is a protease. Exemplary proteases include Factor X, Factor VII, Factor IX or kallikrein.

In another example, a protein of the present disclosure binds to a receptor or a membrane associated protein. Exemplary antigens include, G-protein coupled receptors (such as, CXCR7, CXCR5, CXCR3, C5aR or beta-2-adrenergic receptor) or an ion-channel (such as, a sodium channel or a potassium channel or a calcium channel, preferably, Nicotinic acetylcholine receptor) or a single-span membrane protein (such as a T-cell receptor or a prolactin receptor or a cytokine receptor (e.g., an IL-21-receptor) or a MHC class 1 or a MHC class 2 or CD4 or CD8).

In a further example, a protein of the present disclosure binds to one or more of interferon alpha receptor 1 (IFNAR1), angipoietin-2, IL-4Rα, IL-33, CXCL13, receptor for advanced glycation end products (RAGE), ICOS, IgE, interferon α, IL-6, IL-6 receptor, EphB4, CD19, GM-CSF receptor, CD22, IL-22, EphA2, IL-13, high mobility group protein 1 (HMG1), anaplastic lymphoma kinase (ALK), an integrin (e.g., Integrin αVβ3), Eph receptor, IL-9, EphA4, PC-cell-derived growth factor (PCDGF), nerve growth factor (NGF), insulin-like growth factor (IGF), platelet-derived growth factor (PDGF), platelet-derived growth factor receptor (PDGFR e.g., PDGFRα or PDGFRβ) or IL-5.

Exemplary antibodies from which a protein of the present disclosure can be derived will be apparent to the skilled artisan and include those listed hereinabove.

Exemplary bispecific proteins may bind to two different epitopes of the antigen of interest. Other such proteins may combine one antigen binding site with a binding site for another protein. Alternatively, an anti-antigen of interest region may be combined with a region which binds to a triggering molecule on a leukocyte such as a T-cell receptor molecule (e.g., CD3), or Fc receptors for IgG (FcγR), such as FcγRI (CD64), FcγRII (CD32) and/or FcγRIII (CD16), so as to focus and localize cellular defence mechanisms to the cells expressing the antigen of interest. Bispecific proteins may also be used to localize cytotoxic agents to cells which express the antigen of interest. These proteins possess a region that binds the antigen of interest and a region which binds the cytotoxic agent (e.g., saporin, anti-interferon-α, vinca alkaloid, ricin A chain, methotrexate or radioactive isotope hapten). WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

### Pharmaceutical Compositions and Methods of Treatment

The proteins of the present disclosure (syn. Active ingredients) are useful for parenteral, topical, oral, or local administration, aerosol administration, or transdermal administration for prophylactic or for therapeutic treatment. The pharmaceutical compositions can be administered in a variety of unit dosage forms depending upon the method of administration. For example, unit dosage forms suitable for oral administration include powder, tablets, pills, capsules and lozenges or by parenteral administration. It is recognized that the pharmaceutical compositions of this disclosure, when administered orally, should be protected from digestion. This is typically accomplished either by complexing the proteins with a composition to render it resistant to acidic and enzymatic hydrolysis or by packaging the compound in an appropriately resistant carrier such as a liposome. Means of protecting proteins from digestion are known in the art.

Typically, a therapeutically effective amount of the protein will be formulated into a composition for administration to a subject. The phrase “a therapeutically effective amount” refers to an amount sufficient to promote, induce, and/or enhance treatment or other therapeutic effect in a subject. As will be apparent, the concentration of proteins of the present disclosure in these formulations can vary widely, and will be selected primarily based on fluid volumes, viscosities, body weight and the like in accordance with the particular mode of administration selected and the patient's needs. Depending on the type and severity of the disease, a therapeutically effective amount may be about 1 μg/kg to 100 mg/kg (e.g. for 0.1-10 mg/kg) of protein, whether, for example, by one or more separate administrations, or by continuous infusion. A typical daily dosage might range from about 1 μg/kg to 100 mg/kg or more. For repeated administrations over several days or longer, depending on the condition, the treatment is sustained until a desired suppression of disease symptoms occurs. An exemplary dosing regimen comprises administering an initial loading dose of about 4 mg/kg, followed by a weekly maintenance dose of about 2 mg/kg of the protein. Other dosage regimens may be useful. For example, an anti-CD20 antibody such as rituximab is administered at a dose of about 375 mg/m². An anti-VEGF antibody such as bevacizumabis administered at a dose of 5-10 mg/kg. An anti-Her2/neu antibody such as trastuzumab is administered at a loading dose of 4-8 mg/kg and a weekly/fortnightly maintenance dose of 2-6 mg/kg. An anti-TNFα antibody such as adalimumabis administered at a dose of about 400 mg per week to treat rheumatoid arthritis, or at a loading dose of 160 mg for the first week and a maintenance dose of 40 mg per week, or for psoriasis a loading dose of 80 mg and a maintenance dose of 40 mg per week. The progress of therapy is easily monitored by conventional techniques and assays.

Suitable dosages of proteins of the present disclosure will vary depending on the specific protein, the condition to be diagnosed/treated/prevented and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from cell culture assays or animal studies are used, wherein a suitable dose is within a range of circulating concentrations that include the ED50 of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC50 (i.e., the concentration of the compound which achieves a half-maximal inhibition of symptoms) as determined in cell culture. Such information can be used to more accurately determine useful doses in humans. Levels in plasma maybe measured, for example, by high performance liquid chromatography.

Alternatively, the protein of the present disclosure is formulated at a concentrated dose that is diluted to a therapeutically effective dose prior to administration to a subject.

The compositions of this disclosure are particularly useful for parenteral administration, e.g., formulated for injection via the intravenous, intramuscular, sub-cutaneous, transdermal, or other such routes, including peristaltic administration and direct instillation into a tumour or disease site (intracavity administration). The compositions for administration will commonly comprise a solution of the proteins of the present disclosure dissolved in a pharmaceutically acceptable carrier, preferably an aqueous carrier. A variety of aqueous carriers can be used, e.g., buffered saline and the like. Other exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

Techniques for preparing pharmaceutical compositions are generally known in the art as exemplified by Remington's Pharmaceutical Sciences, 16^{th} Ed. Mack Publishing Company, 1980.

WO2002/080967 describes compositions and methods for administering aerosolized compositions comprising proteins for the treatment of, e.g., asthma, which are also suitable for administration of protein of the present disclosure.

A protein of the present disclosure may be combined in a pharmaceutical combination, formulation, or dosing regimen as combination therapy, with a second compound. The second compound of the pharmaceutical combination formulation or dosing regimen preferably has complementary activities to the protein of the combination such that they do not adversely affect each other.

The second compound may be a chemotherapeutic agent, cytotoxic agent, cytokine, growth inhibitory agent, anti-hormonal agent, and/or cardioprotectant. Such molecules are suitably present in combination in amounts that are effective for the purpose intended. A pharmaceutical composition containing a protein of the present disclosure may also have a therapeutically effective amount of a chemotherapeutic agent such as a tubulin-forming inhibitor, a topoisomerase inhibitor, or a DNA binder.

Pharmaceutical “slow release” capsules or compositions may also be used. Slow release formulations are generally designed to give a constant drug level over an extended period and may be used to deliver compounds of the present disclosure.

The present disclosure also provides a method of treating or preventing a condition in a subject, the method comprising administering a therapeutically effective amount of a protein of the present disclosure to a subject in need thereof.

As used herein, the terms “preventing”, “prevent” or “prevention” in the context of preventing a condition include administering an amount of a protein described herein sufficient to stop or hinder the development of at least one symptom of a specified disease or condition.

As used herein, the terms “treating”, “treat” or “treatment” include administering a therapeutically effective amount of an inhibitor(s) and/or agent(s) described herein sufficient to reduce or eliminate at least one symptom of a specified disease or condition.

As used herein, the term “subject” shall be taken to mean any animal including humans, preferably a mammal. Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). Preferably the mammal is a human or primate. More preferably the mammal is a human.

As used herein, a “condition” is a disruption of or interference with normal function, and is not to be limited to any specific condition, and will include diseases or disorders. In an example, the condition is a cancer or an autoimmune or inflammatory disorder.

Exemplary cancers include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Preferably a cancer is breast cancer or lung cancer or ovarian cancer or prostate cancer.

Inflammatory or autoimmune conditions are conditions caused by the reactions of immunoglobulins or T cell receptors to antigens. These conditions include autoimmune diseases and hypersensitivity responses (e.g. Type I: anaphylaxis, hives, food allergies, asthma; Type II: autoimmune haemolytic anaemia, blood transfusion reactions; Type III: serum sickness, necrotizing vasculitis, glomerulonephritis, rheumatoid arthritis, lupus; Type IV: contact dermatitis, graft rejection). Autoimmune diseases include rheumatologic disorders (such as, for example, rheumatoid arthritis, Sjogren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, rheumatoid arthritis, ulcerative colitis, ANCA-associated vasculitis, lupus, multiple sclerosis, Sjogren's syndrome, Graves' disease, IDDM, pernicious anemia, thyroiditis, and glomerulonephritis.

In another example, an inflammatory condition is a condition involving neutrophils, monocytes, mast cells, basophils, eosinophils, macrophages where cytokine release, histamine release, oxidative burst, phagocytosis, release of other granule enzymes and chemotaxis occur. Hypersensitivity responses (described above) can also be regarded as inflammatory diseases (acute or chronic) since they often involve complement activation and recruitment/infiltration of various leukocytes such as neutrophils, mast cells, basophils, etc.

The compositions of the present disclosure will be administered in a manner compatible with the dosage formulation and in such amount as is therapeutically/prophylactically effective. Formulations are easily administered in a variety of manners, e.g., by ingestion or injection or inhalation.

Other therapeutic regimens may be combined with the administration of a protein of the present disclosure. The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities.

Prior to therapeutic use, a protein of the present disclosure is preferably tested in vitro and/or in vivo, e.g., as described below.

In Vitro Testing

In one example, a protein of the present disclosure binds to an antigen, even if conjugated to a compound. In the case of proteins derived from pre-existing proteins (e.g., antibodies), the protein of the present disclosure may bind to the antigen at least as well as the protein from which it is derived. Alternatively, the protein of the present disclosure binds to the antigen with at least about 10% or 20% or 30% or 40% or 50% or 60% or 70% or 80% or 90% of the affinity or avidity of the protein from which it is derived or a form of the protein lacking the negatively charged residues.

Exemplary methods for determining binding affinity of a protein include a simple immunoassay showing the ability of the protein to block an antibody to a target antigen, e.g., a competitive binding assay. Competitive binding is determined in an assay in which the protein under test inhibits specific binding of a reference protein to a common antigen. Numerous types of competitive binding assays are known, for example, solid phase direct or indirect radioimmunoassay (RIA), solid phase direct or indirect enzyme immunoassay (EIA), sandwich competition assay (see Stahli et al., 1983); solid phase direct biotin-avidin EIA (see Kirkland et al., 1986); solid phase direct labeled assay, solid phase direct labeled sandwich assay (see Harlow and Lane, 1988); solid phase direct biotin-avidin EIA (Cheung et al., 1990); or direct labeled RIA (Moldenhauer et al., 1990). Typically, such an assay involves the use of purified antigen bound to a solid surface or cells bearing either of these, an unlabeled test protein and a labeled reference protein. Competitive inhibition is measured by determining the amount of label bound to the solid surface or cells in the presence of the test protein

The present disclosure also encompasses methods for testing the activity of a protein of the present disclosure. Various assays are available to assess the activity of a protein of the present disclosure in vitro. For example, a protein of the present disclosure is administered to a cell or population thereof to determine whether or not it can bind to said cell and/or be internalized by said cell. Such an assay is facilitated by labeling the protein of the present disclosure with a detectable label (i.e., producing a conjugate), however this is not essential since the protein of the present disclosure can also be detected with a labeled protein. Such an assay is useful for assessing the ability of a protein of the present disclosure to deliver a compound (i.e., a payload) to a cell and/or its utility in imaging. Preferably the cell expresses an antigen to which the protein of the present disclosure binds and more preferably is a cell line or primary cell culture of a cell type that it desired to be detected or treated.

Generally, the cytotoxic or cytostatic activity of a protein of the present disclosure, e.g. conjugated to a cytotoxic molecule is measured by: exposing cells expressing an antigen to which the protein of the present disclosure binds to the protein of the present disclosure; culturing the cells for a suitable period for the protein to exert a biological effect, e.g., from about 6 hours to about 5 days; and measuring cell viability, cytotoxicity and/or cell death. Cell-based in vitro assays useful for measure viability (proliferation), cytotoxicity, and cell death are known in the art.

For example, the CellTiter-Glo® Luminescent Cell Viability Assay is a commercially available (Promega Corp., Madison, Wis.) homogeneous assay method based on the recombinant expression of Coleoptera luciferase (U.S. Pat. Nos. 5,583,024; 5,674,713 and 5,700,670). This cell proliferation assay determines the number of viable cells in culture based on quantitation of the ATP present in a cell, an indicator of metabolically active cells. Alternatively, cell viability is assayed using non-fluorescent resazurin, which is added to cells cultured in the presence of a protein of the present disclosure. Viable cells reduce resazurin to red-fluorescent resorufin, easily detectable, using, for example microscopy or a fluorescent plate reader. Kits for analysis of cell viability are available, for example, from Molecular Probes, Eugene, Oreg., USA.

Other assays for cell viability include determining incorporation of ³H-thymidine or ¹⁴C-thymidine into DNA as it is synthesized (i.e., to determine DNA synthesis associated with cell division). In such an assay, a cell is incubated in the presence of labeled thymidine for a time sufficient for cell division to occur. Following washing to remove any unincorporated thymidine, the label (e.g. the radioactive label) is detected, e.g., using a scintilation counter. Alternative assays for determining cellular proliferation, include, for example, measurement of DNA synthesis by BrdU incorporation (by ELISA or immunohistochemistry, kits available from Amersham Pharmacia Biotech).

Exemplary assays for detecting cell death include APOPTEST (available from Immunotech) stains cells early in apoptosis, and does not require fixation of the cell sample (Martin et al. 1994). This method utilizes an annexin V antibody to detect cell membrane re-configuration that is characteristic of cells undergoing apoptosis. Apoptotic cells stained in this manner can then be sorted either by fluorescence activated cell sorting (FACS), ELISA or by adhesion and panning using immobilized annexin V antibodies. Alternatively, a terminal deoxynucleotidyl transferase-mediated biotinylated UTP nick end-labeling (TUNEL) assay is used to determine the level of cell death. The TUNEL assay uses the enzyme terminal deoxynucleotidyl transferase to label 3′-OH DNA ends, generated during apoptosis, with biotinylated nucleotides. The biotinylated nucleotides are then detected by using streptavidin conjugated to a detectable marker. Kits for TUNEL staining are available from, for example, Intergen Company, Purchase, N.Y.

In vivo Stability of a protein of the present disclosure can also be assessed or predicted by exposing a protein of the present disclosure to serum and/or cells and subsequently isolating the protein of the present disclosure using, for example, immunoaffinity purification. A reduced amount of recovered protein of the present disclosure indicates that the protein of the present disclosure is degraded in serum or when exposed to cells.

In another example, the ability of the protein of the present disclosure to block binding of a ligand to a receptor is assessed using a standard radio-immunoassay or fluorescent-immunoassay.

The ability of a protein of the present disclosure to agonize or antagonize a receptor can also be assessed by determining signalling of the receptor in the presence or absence of the protein.

In Vivo Testing

A protein of the present disclosure can also be tested for its stability and/or efficacy in vivo. For example, the protein of the present disclosure is administered to a subject and the serum levels of the protein is detected over time, e.g., using an ELISA or by detecting a detectable label conjugated to the protein. This permits determination of the in vivo stability of the protein of the present disclosure.

A protein of the present disclosure can also be administered to an animal model of a human disease and its effect on a symptom thereof determined. The skilled artisan will be readily able to determine a suitable model based on the antigen to which the protein of the present disclosure binds. Exemplary models of, for example, human cancer are known in the art. For example, mouse models of breast cancer include mice overexpressing fibroblast growth factor 3 (Muller et al., 1990); TGF-alpha (Matsui et al, 1990); erbB2 (Guy, et al., 1992); or transplantation of human breast cancer cells into SCID mice. Models of ovarian cancer include transplantation of ovarian cancer cells into mice (e.g., as described in Roby et al., 2000); transgenic mice chronically secreting luteinising hormone (Risma et al., 1995); or Wx/Wv mice. Mouse models of prostate cancer are also known in the art and include, for example, models resulting from enforced expression of SV40 early genes (e.g., the TRAMP model that utilizes the minimal rat probasin promoter to express the SV40 early genes or transgenic mice using the long probasin promoter to express large T antigen, collectively termed the ‘LADY’ model or mice expressing c-myc or Bcl-2 or Fgf8b or expressing dominant negative TGFβ (see, Matusik et al., 2001, for a review of transgenic models of prostate cancer).

A protein of the present disclosure can also be administered to an animal model of a disease other than cancer, e.g., NOD mice to test their ability to suppress, prevent, treat or delay diabetes (e.g., as described in Tang et al., 2004) and/or to a mouse model of GVHD (e.g., as described in Trenado, 2002) and/or to a mouse model of psoriasis (e.g., Wang et al. 2008) and/or to a model of rheumatoid arthritis e.g., a SKG strain of mouse (Sakaguchi et al.), rat type II collagen arthritis model, mouse type II collagen arthritis model or antigen induced arthritis models in several species (Bendele, 2001)) and/or a model of multiple sclerosis (for example, experimental autoimmune encephalomyelitis (EAE; Bradl and Linington, 1996)) and/or inflammatory airway disease (for example, OVA challenge or cockroach antigen challenge (Chen et al. 2007) and/or models of inflammatory bowel disease (e.g., dextran sodium sulphate (DSS)-induced colitis or Muc2 deficient mouse model of colitis (Van der Sluis et al. 2006).

### Diagnostic/Prognostic Methods

In one example, the present disclosure provides methods for diagnosing or prognosing a condition.

As used herein, the term “diagnosis”, and variants thereof such as, but not limited to, “diagnose”, “diagnosed” or “diagnosing” includes any primary diagnosis of a clinical state or diagnosis of recurrent disease.

“Prognosis”, “prognosing” and variants thereof as used herein refer to the likely outcome or course of a disease, including the chance of recovery or recurrence.

In one example, the method comprises determining the amount of an antigen in a sample. Thus, the proteins of the present disclosure have utility in applications such as cell sorting (e.g., flow cytometry, fluorescence activated cell sorting), for diagnostic or research purposes. For example, a sample is contacted with a protein of the present disclosure for a time and under conditions sufficient for it to bind to an antigen and form a complex and the complex is then detected or the level of complex is determined. For these purposes, the proteins can be labeled or unlabeled. The proteins can be directly labeled, e.g., using a label described herein. When unlabeled, the proteins can be detected using suitable means, as in agglutination assays, for example. Unlabeled antibodies or fragments can also be used in combination with another (i.e., one or more) suitable reagent which can be used to detect a protein, such as a labeled antibody (e.g., a second antibody) reactive with the protein or other suitable reagent (e.g., labeled protein A).

Preferably, a protein of the present disclosure is used in an immunoassay. Preferably, using an assay selected from the group consisting of, immunohistochemistry, immunofluorescence, enzyme linked immunosorbent assay (ELISA), fluorescence linked immunosorbent assay (FLISA) Western blotting, RIA, a biosensor assay, a protein chip assay and an immunostaining assay (e.g. immunofluorescence).

Standard solid-phase ELISA or FLISA formats are particularly useful in determining the concentration of a protein from a variety of samples.

In one form, such an assay involves immobilizing a biological sample onto a solid matrix, such as, for example a polystyrene or polycarbonate microwell or dipstick, a membrane, or a glass support (e.g. a glass slide). A protein of the present disclosure that specifically binds to an antigen of interest is brought into direct contact with the immobilized sample, and forms a direct bond with any of its target antigen present in said sample. This protein of the present disclosure is generally labeled with a detectable reporter molecule, such as for example, a fluorescent label (e.g. FITC or Texas Red) or a fluorescent semiconductor nanocrystal (as described in U.S. Pat. No. 6,306,610) in the case of a FLISA or an enzyme (e.g. horseradish peroxidase (HRP), alkaline phosphatase (AP) or β-galactosidase) in the case of an ELISA, or alternatively a labeled antibody can be used that binds to the protein of the present disclosure. Following washing to remove any unbound protein the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal) in the case of an enzymatic label. Such ELISA or FLISA based systems are particularly suitable for quantification of the amount of a protein in a sample, by calibrating the detection system against known amounts of a protein standard to which the protein binds, such as for example, an isolated and/or recombinant protein or immunogenic fragment thereof or epitope thereof.

In another form, an ELISA or FLISA comprises immobilizing a protein of the present disclosure or an antibody that binds to an antigen of interest on a solid matrix, such as, for example, a membrane, a polystyrene or polycarbonate microwell, a polystyrene or polycarbonate dipstick or a glass support. A sample is then brought into physical contact with said protein of the present disclosure or antibody, and the protein to which said compound binds is bound or ‘captured’. The bound protein is then detected using a labeled protein of the present disclosure that binds to a different protein or a different site in the same antigen. Alternatively, a third labeled antibody can be used that binds the second (detecting) protein.

### Imaging Methods

As will be apparent to the skilled artisan from the foregoing, the present disclosure also contemplates imaging methods using a protein of the present disclosure. For imaging, protein of the present disclosure is conjugated to a detectable label, which can be any molecule or agent that can emit a signal that is detectable by imaging. For example, the detectable label may be a protein, a radioisotope, a fluorophore, a visible light emitting fluorophore, infrared light emitting fluorophore, a metal, a ferromagnetic substance, an electromagnetic emitting substance a substance with a specific magnetic resonance (MR) spectroscopic signature, an X-ray absorbing or reflecting substance, or a sound altering substance.

The protein of the present disclosure can be administered either systemically or locally to the tumor, organ, or tissue to be imaged, prior to the imaging procedure. Generally, the protein is administered in doses effective to achieve the desired optical image of a tumour, tissue, or organ. Such doses may vary widely, depending upon the particular protein employed, the tumour, tissue, or organ subjected to the imaging procedure, the imaging equipment being used, and the like.

In some embodiments of the disclosure, the protein of the present disclosure is used as in vivo optical imaging agents of tissues and organs in various biomedical applications including, but not limited to, imaging of tumors, tomographic imaging of organs, monitoring of organ functions, coronary angiography, fluorescence endoscopy, laser guided surgery, photoacoustic and sonofluorescence methods, and the like. Exemplary diseases, e.g., cancers, in which a protein of the present disclosure is useful for imaging are described herein and shall be taken to apply mutatis mutandis to the present example of the disclosure. In one example, a protein conjugate of the disclosure is useful for the detection of the presence of tumors and other abnormalities by monitoring where a particular protein of the present disclosure is concentrated in a subject. In another example, the protein of the present disclosure is useful for laser-assisted guided surgery for the detection of micro-metastases of tumors upon laparoscopy. In yet another example, the protein of the present disclosure is useful in the diagnosis of atherosclerotic plaques and blood clots.

Examples of imaging methods include magnetic resonance imaging (MRI), MR spectroscopy, radiography, CT, ultrasound, planar gamma camera imaging, single-photon emission computed tomography (SPECT), positron emission tomography (PET), other nuclear medicine-based imaging, optical imaging using visible light, optical imaging using luciferase, optical imaging using a fluorophore, other optical imaging, imaging using near infrared light, or imaging using infrared light.

Certain examples of the methods of the present disclosure further include imaging a tissue during a surgical procedure on a subject.

A variety of techniques for imaging are known to those of ordinary skill in the art. Any of these techniques can be applied in the context of the imaging methods of the present disclosure to measure a signal from the detectable label. For example, optical imaging is one imaging modality that has gained widespread acceptance in particular areas of medicine. Examples include optical labeling of cellular components, and angiography such as fluorescein angiography and indocyanine green angiography. Examples of optical imaging agents include, for example, fluorescein, a fluorescein derivative, indocyanine green, Oregon green, a derivative of Oregon green derivative, rhodamine green, a derivative of rhodamine green, an eosin, an erytlirosin, Texas red, a derivative of Texas red, malachite green, nanogold sulfosuccinimidyl ester, cascade blue, a coumarin derivative, a naphthalene, a pyridyloxazole derivative, cascade yellow dye, dapoxyl dye.

Gamma camera imaging is contemplated as a method of imaging that can be utilized for measuring a signal derived from the detectable label. One of ordinary skill in the art would be familiar with techniques for application of gamma camera imaging. In one embodiment, measuring a signal can involve use of gamma-camera imaging of an ¹¹¹In or ^{99m}Tc conjugate, in particular ¹¹¹In-octreotide or ^{99m}Tc-somatostatin analogue.

Computerized tomography (CT) is contemplated as an imaging modality in the context of the present disclosure. By taking a series of X-rays from various angles and then combining them using computer software, CT makes it possible to construct a three-dimensional image of any part of the body. A computer is programmed to display two-dimensional slices from any angle and at any depth. The slices may be combined to build three-dimensional representations.

In CT, intravenous injection of a radiopaque contrast agent conjugated to a protein of the present disclosure, which binds to an antigen of interest can assist in the identification and delineation of tissue masses (e.g., soft tissue masses) when initial CT scans are not diagnostic. Similarly, contrast agents aid in assessing the vascularity of a soft tissue lesion. For example, the use of contrast agents may aid the delineation of the relationship of a tumor and adjacent vascular structures.

CT contrast agents include, for example, iodinated contrast media. Examples of these agents include iothalamate, iohexol, diatrizoate, iopamidol, ethiodol, and iopanoate. Gadolinium agents have also been reported to be of use as a CT contrast agent, for example, gadopentate.

Magnetic resonance imaging (MRI) is an imaging modality that uses a high-strength magnet and radio-frequency signals to produce images. In MRI, the sample to be imaged is placed in a strong static magnetic field and excited with a pulse of radio frequency (RF) radiation to produce a net magnetization in the sample. Various magnetic field gradients and other RF pulses then act to code spatial information into the recorded signals. By collecting and analyzing these signals, it is possible to compute a three-dimensional image which, like a CT image, is normally displayed in two-dimensional slices. The slices may be combined to build three-dimensional representations.

Contrast agents used in MRI or MR spectroscopy imaging differ from those used in other imaging techniques. Examples of MM contrast agents include gadolinium chelates, manganese chelates, chromium chelates, and iron particles. For example, a protein of the present disclosure is conjugated to a compound comprising a chelate of a paramagnetic metal selected from the group consisting of scandium, titanium, vanadium, chromium, manganese, iron, cobalt, nickel, copper, molybdenum, ruthenium, cerium, indium, praseodymium, neodymium, promethium, samarium, europium, gadolinium, terbium, dysprosium, holmium, erbium, thulium, and ytterbium. A further example of imaging agents useful for the present disclosure is halocarbon-based nanoparticle such as PFOB or other fluorine-based MRI agents. Both CT and MRI provide anatomical information that aid in distinguishing tissue boundaries and vascular structure.

Imaging modalities that provide information pertaining to information at the cellular level, such as cellular viability, include positron emission tomography (PET) and single-photon emission computed tomography (SPECT). In PET, a patient ingests or is injected with a radioactive substance that emits positrons, which can be monitored as the substance moves through the body.

The major difference between PET and SPECT is that instead of a positron-emitting substance, SPECT uses a radioactive tracer that emits high-energy photons. SPECT is valuable for diagnosing multiple illnesses including coronary artery disease, and already some 2.5 million SPECT heart studies are done in the United States each year.

For PET, a protein of the present disclosure is commonly labeled with positron-emitters such as ¹¹C, ¹³N, ¹⁵O, ¹⁸F, ⁸²Rb, ⁶²Cu, and ⁶⁸Ga. Proteins of the present disclosure are labeled with positron emitters such as 99mTc, ²⁰¹Tl, and ⁶⁷Ga, ¹¹¹In for SPECT.

Non-invasive fluorescence imaging of animals and humans can also provide in vivo diagnostic information and be used in a wide variety of clinical specialties. For instance, techniques have been developed over the years including simple observations following UV excitation of fluorophores up to sophisticated spectroscopic imaging using advanced equipment (see, e.g., Andersson-Engels et al, 1997). Specific devices or methods known in the art for the in vivo detection of fluorescence, e.g., from fluorophores or fluorescent proteins, include, but are not limited to, in vivo near-infrared fluorescence (see, e.g., Frangioni, 2003), the Maestro™ in vivo fluorescence imaging system (Cambridge Research & Instrumentation, Inc.; Woburn, Mass.), in vivo fluorescence imaging using a flying-spot scanner (see, e.g., Ramanujam et al, 2001), and the like.

Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

In some examples, an imaging agent is tested using an in vitro or in vivo assay prior to use in humans, e.g., using a model described herein.

### Articles of Manufacture

The present disclosure also provides an article of manufacture, or “kit”, containing a protein of the present disclosure. The article of manufacture can comprise a container and a label or package insert on or associated with the container, e.g., providing instructions to use the protein of the present disclosure in a method described herein according to any embodiment. Suitable containers include, for example, bottles, vials, syringes, blister pack, etc. The containers may be formed from a variety of materials such as glass or plastic. The container holds a protein of the present disclosure composition and may have a sterile access port (for example the container may be an intravenous solution bag or a vial having a stopper pierceable by a hypodermic injection needle). Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. The kit may also or alternatively comprise reagents for detecting a protein of the present disclosure and/or for conjugating to a protein of the present disclosure.

The present disclosure is described further in the following non-limiting examples.

### Example 1

### Materials and Methods

Based on the HEL4 V_{H} domain, as described in Jespers et al., 2004, a mutational approach was used to identify mutations that render this domain aggregation-resistant. The approach was based on generating chimeras between HEL4 and DP47 (the aggregation-prone germline V_{H} from which HEL4 is derived).

1.1 Generation of Mutant V_{H} and scFv

Mutants of human variable domains were generated using the method as described by Zoller and Smith (1987), with modifications introduced by Kunkel et al. (1987). For this purpose, synthetic oligonucleotides encoding the desired mutations were annealed to a uracil-containing single-stranded template DNA (dU-ssDNA), enzymatically extended and ligated to form covalently closed circular DNA. Template was generated by the cloning of DNA fragments encoding a single human heavy chain variable (V_{H}) domain (V3-23/DP-47) into the phage display vector, FdMyc, using ApaLI and NotI sites. Covalently closed circular DNA was transformed by electroporation into the ung⁺E. coli strain TG1, causing preferential destruction of non-mutated dU-ssDNA. The sequences of the constructed mutants were confirmed by DNA sequence analysis.

For the generation of scFv mutants, DNA fragments encoding a single V_{κ} domain (SEQ ID NO: 3) and synthetic linker region (SEQ ID NO: 4) were cloned into the corresponding FdMyc constructs using XhoI and NotI cloning sites. The sequences of the constructed mutants were confirmed by DNA sequence analysis.

### 1.2 Phage ELISA for Aggregation-Resistance (“Heat/Cool Assay”)

The aggregation-resistance of clones was analyzed by measuring retention of signal after heat incubation in a phage ELISA format (McCafferty et al., 1990; Jespers et al., 2004). Wells of a Nunc Maxisorp Immuno-plate were coated overnight with protein A at a concentration of about 5 μg/ml in phosphate-buffered saline (PBS). The plate was washed once with PBS and blocked with about 4% (w/v) milk powder diluted in PBS (MPBS). Single colonies were picked from agar plates and grown overnight in 2×TY medium (containing about 16 g/L tryptone; about 10 g/L yeast extract; about 5 g/L NaCl, pH 7.0) supplemented with about 15 μg/ml tetracycline shaking at about 30° C. Cells were removed by centrifugation and phages were biotinylated directly in the culture supernatant by adding biotin-PEO₄-N-hydroxysuccinimide (Pierce; about 50 μM final concentration). For heat selection, supernatant was first incubated at about 80° C. for about 10 min and then at about 4° C. for about 10 min. Supernatant was added to the blocked ELISA wells. After three washes with PBS, bound phage particles were detected using an Extravidin-HRP conjugate (Sigma) and 3,3′,5,5′-tetramethylbenzidine (TMB) substrate. Absorbance was calculated by subtracting measurements at 450 and 650 nm.

### 1.3 Generation of V_{H} Libraries, ‘Garvan-IA’ and ‘IB’

Two V_{H} libraries were constructed in which CDR3 of the HEL4 V_{H} clone was randomized using the method described by Zoller and Smith, 1987, with modifications introduced by Kunkel et al., 1987. For this purpose, synthetic oligonucleotides encoding the desired mutations were annealed to a uracil-containing single-stranded template DNA (dU-ssDNA), enzymatically extended and ligated to form covalently closed circular DNA. Template was generated by cloning of DNA fragments encoding a single human V_{H} domain (HEL4) into the phage display vector, FdMyc, using ApaLI and NotI sites. Covalently closed circular DNA was transformed by electroporation into the ung⁺E. coli strain TG1, causing preferential destruction of non-mutated dU-ssDNA. The ‘Garvan-IA’ library was generated by randomization of 7 amino acid residues at positions 96, 97, 98, 99, 100, 100a, and 100b (numbering according to Kabat et al., 1992) using the degenerate DVK codon at all 7 positions. Likewise, the ‘Garvan-IB’ library was randomized at positions 95, 96, 97, 98, 99, 100, 100a, 100b, and 100c, where 95 and 100c were randomized using the degenerate NNK codon in the encoding nucleic acid, with the remaining positions randomized using the degenerate DVK coding in the encoding nucleic acid. The resulting library sizes were about 1.1×10⁹ colonies for Garvan-IA, and about 2.2×10⁹ colonies for Garvan-IB.

### 1.4 Phage Display Selection of Anti-hTNF and Anti-mIL-21 V_{H} Clones

Phage from the naïve Garvan-IA and IB libraries (in the FdMyc vector) were cycled through 2 rounds of selection against biotinylated recombinant hTNF (human tumor necrosis factor; Peprotech) or mIL-21, essentially as previously described (Lee et al., 2007). After two rounds of selections, regions encoding V_{H} domains were PCR amplified from phage DNA preparations using the primers, 5′-ACGCGTCGACGCAGGTGCAGCTGTTGG-3′(SEQ ID NO: 16) and 5′-CTGTTAGGATCCGCTCGAGACGGTGACCAG-3′ (SEQ ID NO: 17). The PCR products were digested with SalI and BamHI restriction enzymes and cloned into the corresponding sites of a modified pET12a expression plasmid (New England Biolabs) encoding c-Myc and His₆ tags. The resulting ligation reactions were transformed into E. coli strain BL21-Gold (Stratagene) and 192 colonies of each antigen selection were grown in 2×TY broth supplemented with about 4% glucose and ampicillin (about 100 μg/mL) for about 18 hr at about 37° C., shaking at about 250 rpm. The overnight cultures were used to inoculate fresh 2×TY media supplemented with about 0.1% glucose and ampicillin (about 100 μg/mL) and grown to an OD_{600 nm} of about 0.5, at which point isopropyl β-D-1-thiogalactopyranoside (IPTG) was added to a final concentration of about 1 mM to induce soluble V_{H} expression. Cultures were grown for about 18 hr at about 30° C., shaking at about 250 rpm. Cells were removed by centrifugation and culture supernatants were tested for antigen binding by ELISA.

For ELISAs, wells of a Nunc Maxisorp Immuno-plate were coated overnight with antigen at a concentration of about 5 μg/ml in PBS. The plate was washed once with PBS and blocked with about 4% (w/v) milk powder diluted in PBS. Supernatant was added to the blocked ELISA wells. After three washes with PBS, bound antibody domains were detected using a biotinylated chicken-anti-c-Myc antibody (Immunology Consultants Laboratory) for hTNF selections or biotinylated mouse anti-c-Myc (Sigma, clone 9E10) for mIL-21 selections, followed by Extravidin-HRP conjugate (Sigma) and 3,3′,5,5′-tetramethylbenzidine (TMB) substrate. Absorbance was calculated by subtracting measurements at 450 and 650 nm.

### 1.5 Specificity ELISA of Isolated V_{H} Clones

The specificity of isolated V_{H} clones, G07 (anti-hTNF; SEQ ID NO: 5) and G11 (anti-mIL-21; SEQ ID NO: 6), was determined by ELISA. Wells of a Nunc Maxisorp Immuno-plate were coated over night with about 5 μg/mL of either recombinant human TNF, mouse TNF, human IL-21, mouse IL-21, beta galactosidase, human prolactin receptor, streptavidin, and neutravidin. The plate was washed once with PBS and blocked with about 4% (w/v) milk powder diluted in PBS buffer. Purified G07 and G11 were added to the plate at about 10 μg/mL diluted in PBS and incubated at room temperature for about 1 hr. After three washes with PBS, V_{H} were detected with either biotinylated chicken anti-c-myc (for G07) or biotinylated mouse anti-c-myc (for G11), followed by the addition of Extravadin-HRP and 3,3′,5,5′-tetramethylbenzidine (TMB) substrate. Absorbance was calculated by subtracting measurements at 450 and 650 nm.

### 1.6 Affinity Measurements of Anti-hTNF and Anti-mIL-21 V_{H} Clones

The affinities of V_{H} clones G07 (anti-hTNF; SEQ ID NO: 5) and G11 (anti-mIL-21; SEQ ID NO: 6) were measured using surface plasmon resonance (using a Biacore machine; GE Healthcare). For this purpose, biotinylated antigen diluted in PBS was injected over a streptavidin (SA) sensor chip (Biacore AB). Serial dilutions of purified V_{H} (with concentrations ranging from about 0.125 to 4 μM) were injected at a flowrate of about 20 μl/min over the flow cell containing the corresponding target antigen. Equilibrium dissociation constants were calculated using the BIAevaluation 4.1 software package (Biacore AB).

### 1.7 Determining Soluble Expression Levels of V_{H} Domains

The soluble expression level of each V_{H} domains was determined using a protein A ELISA in which the concentration of soluble V_{H} of the V_{H} was measured against a standard curve of the same purified V_{H}. DP47, HEL4 and mutant V_{H}s were expressed from a pET12a (New England Biolabs) vector in BL21-GOLD E. coli (Stratagene). After 42 hr, cells were removed by centrifugation and V_{H}s were biotinylated directly in the culture supernatant by adding biotin-PEO₄-N-hydroxysuccinimide (Pierce; 50 μM final concentration). Culture supernatant and biotinylated purified V_{H} of the same mutant at known concentration were added to a Nunc 96-well Maxisorp immunoplate coated overnight with 5 μg/ml Protein A (Sigma) and blocked with 4% MPBS. After three washes with PBST, bound antibody domains were detected using Extravidin-HRP conjugate (Sigma) and TMB substrate. Absorbance was calculated by subtracting measurements at 450 and 650 nm and concentrations of each sample were extrapolated from the standard curve.

### 1.8 Determining Aggregation-Resistance by Size Exclusion Chromatography

Purified V_{H} at 10 μM in PBS were either heated to 80° C. for 10 mins followed by cooling at 4° C. for 10 mins or not treated. Both heated and unheated samples were centrifuged at 16,000×g for 10 mins before 500 μl of each were analyzed on a Superdex-G75 column (Pharmacia) equilibrated with 25 mM sodium phosphate (pH 7.4) containing 125 mM NaCl. The proteins were injected at a volume of 500 μl with a flow rate of 0.5 ml/min. The recovery of each V_{H} mutant was determined by measuring the area under the curve of the heated sample, expressed as percentage of the unheated sample.

### 1.9 Determining Aggregation-Resistance Using Circular Dichroism

The thermal unfolding V_{H} domains was measured by circular dichroism (CD) using a J-815 spectrometer (Jasco) in a quartz cuvette (1 mm path length). Protein samples were at a final concentration of 20 μM in PBS (pH 7.2) and melting curves were obtained by recording the CD signal at 235 nm with a 1 nm bandwith and 1 s integration time while heating the solutions form 20° C. to 80° C. at PC/min. The aggregation-resistance of each sample was tested by cooling the heated protein from 80° C. to 4° C. at 1° C./min.

### 1.10 Measurement of V_{H} Domain Retention by Size-Exclusion Columns

DP47 V_{H} domains containing mutations in CDR1 were expressed and purified as previously described. Each protein sample, at 5 μM in PBS, was heated to 80° C. for 10 mins followed by cooling at 4° C. for 10 mins. Heated samples were centrifuged at 16,000×g for 10 mins before 500 μl of each were analyzed on a Superdex-G75 column (Pharmacia) equilibrated with 25 mM sodium phosphate (pH 7.4) containing 125 mM NaCl. The proteins were injected at a volume of 500 μl with a flow rate of 0.5 ml/min.

### 1.11 Generation of V_{H} Library ‘Garvan-2’

A V_{H} library was constructed in which multiple CDR residues of the HEL4 V_{H} clone were randomized using the method described by Zoller and Smith, 1987, with modifications introduced by Kunkel et al., 1987. For this purpose, synthetic oligonucleotides encoding the desired mutations were annealed to a uracil-containing single-stranded template DNA (dU-ssDNA), enzymatically extended and ligated to form covalently closed circular DNA. Template was generated by cloning of DNA fragments encoding a single human V_{H} domain (HEL4) into the phage display vector, pHEN1. Covalently closed circular DNA was transformed by electroporation into the ung⁺E. coli strain TG1, causing preferential destruction of non-mutated dU-ssDNA. The library was generated by randomization of two amino acid residues at positions 30 and 31 in CDR1 (numbering according to Kabat et al., 1992) using the degenerate KMT codon. Likewise, CDR2 was randomized at positions 50, 52, 55 using the degenerate KMT codon, at position 52a using the degenerate RRT codon and at position 53 using the degenerate SMT codon. Furthermore, positions 29, 94, 100x (with x indicating the position following the C-terminal degenerate position), 101 and 102 of the HEL4 domain were mutated to the corresponding DP47 residues. Positions 95-100a or alternatively positions 95-100c were then further randomized using SOE-PCR mutagenesis essentially as previously described (Higuchi, Krummel et al. 1988). Amino acid residues encoded within the CDR3 design included all 19 naturally occurring amino acids, but excluded cysteine and stop codons. Covalently closed circular DNA was transformed by electroporation into the E. coli strain TG1. The resulting library size included about 4×10⁹ colonies. The Garvan-2 library encodes two or more negative charges, two of which are at positions 32 and 33.

1.12 Phage Display Selection of Anti-hPRLR and Anti-HEL V_{H} clones

Phage from the naïve Garvan-2 library were cycled through multiple rounds of selection, essentially as previously described (Lee et al., 2007). After selection, colonies resulting from each antigen selection were grown in 2×TY broth supplemented with about 4% glucose and ampicillin (about 100 μg/mL) for about 18 hr at about 37° C., shaking at about 250 rpm. The overnight cultures were used to inoculate fresh 2×TY media supplemented with about 0.1% glucose and ampicillin (about 100 μg/mL) and grown to an OD_{600 nm} of about 0.5, at which point isopropyl β-D-1-thiogalactopyranoside (IPTG) was added to a final concentration of about 1 mM to induce soluble V_{H} expression. Cultures were grown for about 18 hr at about 30° C., shaking at about 250 rpm. Cells were removed by centrifugation and culture supernatants were tested for antigen binding by ELISA.

For ELISAs, wells of a Nunc Maxisorp Immuno-plate were coated overnight with antigen at a concentration of about 5 μg/ml in PBS. The plate was washed once with PBS and blocked with about 4% (w/v) milk powder diluted in PBS. Supernatant was added to the blocked ELISA wells. After three washes with PBS, bound antibody domains were detected using a biotinylated chicken-anti-c-Myc antibody (Immunology Consultants Laboratory) or biotinylated mouse anti-c-Myc (Sigma, clone 9E10), followed by Extravidin-HRP conjugate (Sigma) and 3,3′,5,5′-tetramethylbenzidine (TMB) substrate. Absorbance was calculated by subtracting measurements at 450 and 650 nm.

### 1.14 Affinity Measurements of of Anti-hPRLR and Anti-HEL V_{H} Clones

The affinities of V_{H} clones were measured using surface plasmon resonance (using a Biacore2000 instrument; GE Healthcare). For this purpose, biotinylated antigen diluted in PBS was injected over a streptavidin (SA) sensor chip (Biacore AB). Serial dilutions of purified V_{H} were injected over the flow cell containing the corresponding target antigen. Equilibrium dissociation constants were calculated using the BIAevaluation 4.1 software package (Biacore AB).

### Example 2

### Aggregation-Resistance of HEL4/DP47 CDR Chimeras

Experiments were performed to investigate the effects of introducing single HEL4 CDRs (CDR1 or CDR2 or CDR3) into DP47. The HEL4/DP47 CDRs chimeras were constructed and tested for aggregation-resistance, as detailed above (see the Materials and Methods section).

Briefly, phage-displayed V_{H} were heated to 80° C. for 10 min, followed by cooling at 4° C. for 10 min. Correctly folded V_{H} were captured by protein A ELISA and the absorbance signal of the treated sample was calculated as a percentage of the untreated sample.

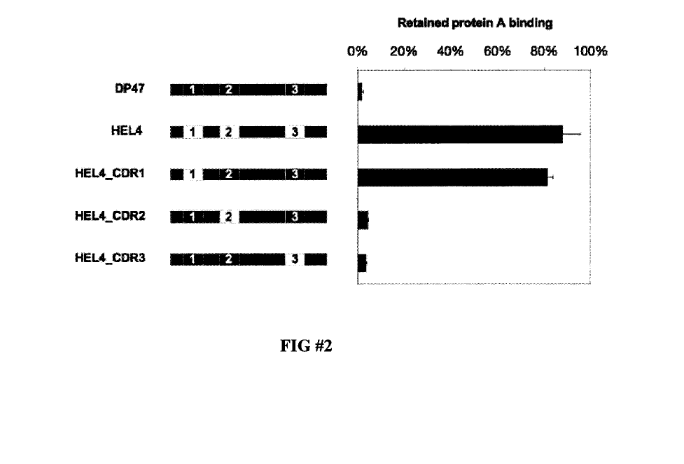

Results are shown in Table 1 and FIG. 2. In summary, the introduction of HEL4-CDR1 conferred considerable aggregation-resistance on the DP47 V_{H} domain. On the other hand, introduction of the HEL4-CDR2 or the HEL4-CDR3 into DP47 had limited effect on the aggregation-resistance of the V_{H} domain.

TABLE 1
Aggregation-resistance of DP47/HEL4 CDR chimeras.

| VH | DP47 | HEL4 | HEL4-CDR1 | HEL4-CDR2 | HEL4-CDR3 |
|---|---|---|---|---|---|
| Retained | 2% | 88% | 81% | 4% | 3% |
| binding to | | | | | |
| protein A | | | | | |

### Example 3

### Mapping of CDR1

### Aggregation-Resistance of DP47 CDR1 Mutants

Experiments were performed to further identify the regions of CDR1 responsible for conferring aggregation-resistance on the DP47 V_{H} domain.

Single amino acid changes in the CDR1 region of the DP47 V_{H} domain, and combinations thereof, were constructed and tested for aggregation-resistance, as detailed above (see the Materials and Methods section). Briefly, phage-displayed V_{H} were heated to 80° C. for 10 min, followed by cooling at 4° C. for 10 min. Correctly folded V_{H} were captured by protein A ELISA and the absorbance signal of the treated sample was calculated as a percentage of the untreated sample.

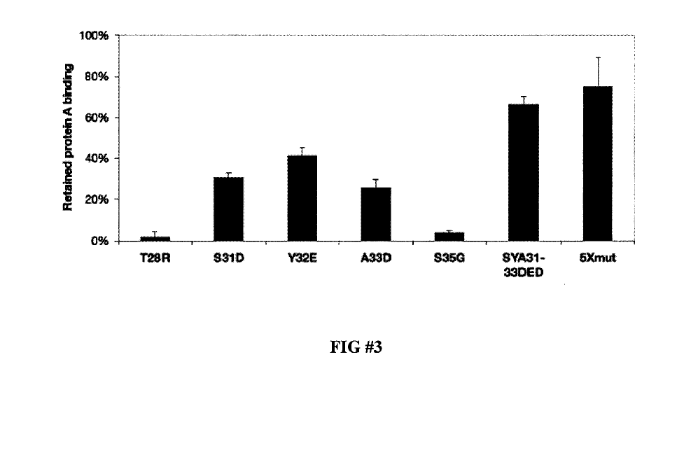

Results are shown in Table 2 and FIG. 3. In summary, introduction of negatively charged amino acids at positions 31 or 32 or 33 of CDR1 resulted in considerable aggregation-resistance of the V_{H} domain. Furthermore, a triple amino acid mutation at 31-33 (SYA31-33DED) resulted in greater aggregation-resistance than observed for single amino acid changes. Other mutations (T28R, S35G) at positions 28 and 35 had little effect on aggregation-resistance. These previously described mutations do not introduce negatively charged amino acids.

TABLE 2
Aggregation-resistance of DP47-CDR1 mutants

| | VH | | | | | | |
|---|---|---|---|---|---|---|---|
| | | | | | | SYA31- | 5X |
| | T28R | S31D | Y32E | A33D | S35G | 33DED | mut |
| Retained binding | 2% | 31% | 41% | 26% | 4% | 67% | 75% |
| to protein A | | | | | | | |

### Example 4

### Aggregation-Resistance of DP47-CDR1 Mutants and Combinations Thereof when Paired with a Common V_{L} Chain (as scFv)

The DP47-CDR1 mutants described above were paired with a common single variable light chain (V_{L}; SEQ ID NO: 3) via a linker (SEQ ID NO: 4) in a scFv format (see the Materials and Methods section for full experimental details). Briefly, phage-displayed V_{H} were heated to 80° C. for 10 min, followed by cooling at 4° C. for 10 min. Correctly folded scFv were captured by protein A ELISA and the absorbance signal of the treated sample was calculated as a percentage of the untreated sample.

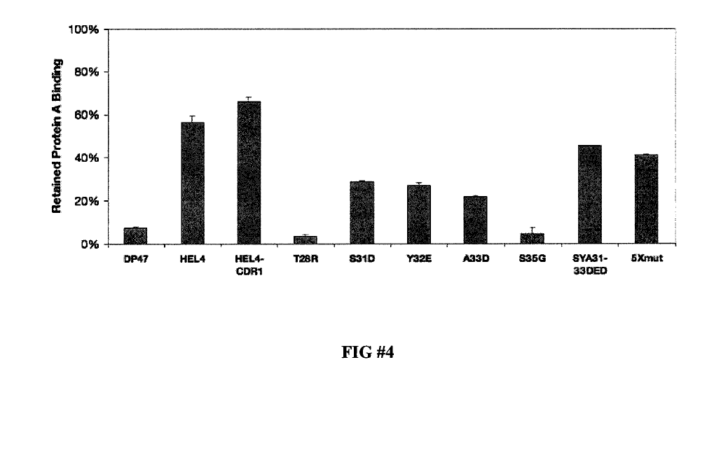

Results are shown in Table 3 and FIG. 4. In summary, DP47-CDR1 mutants in the scFv format showed similar improvements of aggregation-resistance as in the V_{H} format (see Table 2 and FIG. 3). The results show that introduction of negatively charged amino acids at positions 31 or 32 or 33 of CDR1 (or combinations thereof) improve aggregation-resistance of V_{H} when paired with a common V_{L} chain (as scFv). Furthermore, a triple amino acid mutation at 31-33 (SYA31-33DED) resulted in greater aggregation-resistance than observed for single amino acid changes. Other mutations (T28R, S35G) at positions 28 and 35 had little effect on aggregation-resistance. These previously described mutations do not introduce negatively charged amino acids.

TABLE 3
Aggregation-resistance of DP47-CDR1 mutants (V_{H}) coupled to a
single variable light chain (V_{L}) via linker in a scFv format.

| scFv | DP47 | HEL4 | HEL4-CDR1 | T28R | S31D | Y32E | A33D | S35G | SYA31-33DED | 5X mut |
|---|---|---|---|---|---|---|---|---|---|---|
| Retained binding | 8% | 56% | 66% | 4% | 29% | 27% | 22% | 5% | 45% | 41% |
| to protein A | | | | | | | | | | |

### Example 5

### Generations of DP47 V_{H} Constructs Containing Double Mutations in CDR1

Double mutations in the CDR1 region of V_{H} are constructed substantially as described for the single and multiple DP47-CDR1 mutants in the examples above. For example, double mutations at position 32 and 33 positions of CDR1 of V_{H} are introduced. Alternatively, double mutations at positions 31 and 32 of CDR1 of V_{H} are introduced. Alternatively, double mutations at positions 31 and 33 of CDR1 of V_{H} are introduced.

In the above examples of double mutations in the CDR1 region of V_{H}, negatively charged amino acids, such as aspartic acid (D) and/or glutamic acid (E), are introduced at positions 32 and 33, or positions 31 and 32, or positions 31 and 33 of CDR1. The aggregation-resistance of the double mutant DP47 V_{H} domains are measured using the phage “Heat/Cool” assay as described in the Materials and Methods section above. Briefly, phage-displayed antibodies are heated to about 80° C. for about 10 min, followed by cooling at about 4° C. for about 10 min. Correctly folded V_{H} are captured by protein A ELISA and the absorbance signal of the treated sample is calculated as a percentage of the untreated sample.

In some examples, double mutant V_{H} constructs are paired with light chain (V_{L}) to determine the effect of mutations on scFv aggregation-resistance. The aggregation-resistance of mutant scFv is measured using the phage “Heat/Cool” assay as described in the Materials and Methods section above. Briefly, phage-displayed antibodies are heated to about 80° C. for about 10 min, followed by cooling at about 4° C. for about 10 min. Correctly folded scFv are captured by protein A ELISA and the absorbance signal of the treated sample is calculated as a percentage of the untreated sample.

### Example 6

### Aggregation-Resistance of CDR1 Mutants as Purified Proteins

Aggregation-resistance of the set of CDR1 mutants described in the above examples is evaluated in the context of purified V_{H} or V_{H}-V_{L} combinations (i.e. not displayed on phage). For these experiments, mutant V_{H} (or V_{H}-V_{L}) domains are expressed and purified substantially as described above in the Materials and Methods section. Resistance against heat-induced aggregation is studied by circular dichroism (CD) and/or size exclusion chromatography and/or by turbidity analyses. Thermodynamic stabilities are determined by circular dichroism and/or fluorescence spectroscopy.

### Example 7

### Mutation of Existing Antibodies to Introduce the CDR1 Mutations

Existing known monoclonal antibodies, such as, for example, Humira, (also known in the art as adalimumab; V_{H} sequence set forth in SEQ ID NO: 10) and/or Rituxan (also known in the art as Mabthera or rituximab; SEQ ID NO: 11) and/or Herceptin (also known in the art as trastuzumab; SEQ ID NO: 12) and/or Avastin (also known in the art as bevacizumab; SEQ ID NO: 13) are modified by introducing negatively charged amino acids at positions 28 and/or 30 and/or 31 and/or 32 and/or 33 and/or 35. Additionally, negatively charged amino acids may be introduced at positions 26 and/or 39 and/or 40 and/or 50 and/or 52 and/or 52a and/or 53. Characterization of these modified antibodies is performed as V_{H} domains or scFv only (not whole IgG) and comprises the “Heat/Cool” assay, as described above in the Materials and Methods section and/or any one or more of the assays described in Example 6.

### Example 8

### Analysis of V_{H} Mutants in the Context of IgG

Testing of V_{H} mutants is performed in the context of whole IgG. For this purpose, mutant IgGs are expressed and purified. Resistance against aggregation is studied by circular dichroism and/or size exclusion chromatography and/or by turbidity analyses.

### Example 9

### Aggregation-Resistance of Garvan-IA and IB Libraries

Garvan-IA and IB libraries were constructed and isolated as described above (see Materials and Methods section). Aggregation-resistance of naïve clones from the Garvan-IA and IB human V_{H} libraries was investigated. Briefly, phage-displayed antibodies were heated to about 80° C. for about 10 min, followed by cooling at about 4° C. for about 10 min. Correctly folded V_{H} were captured by protein A ELISA and the absorbance signal of the treated sample was calculated as a percentage of the untreated sample.

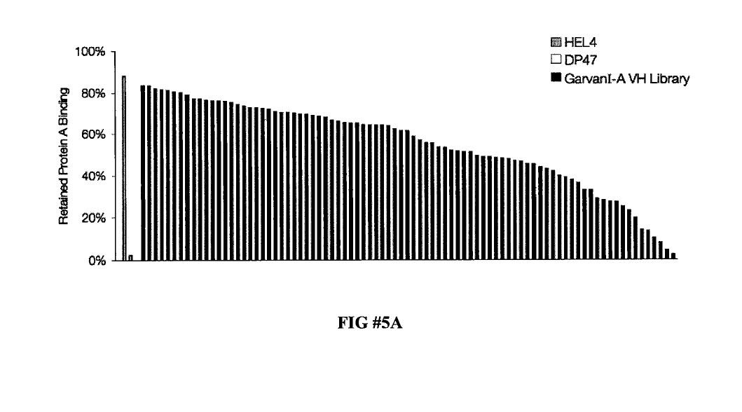

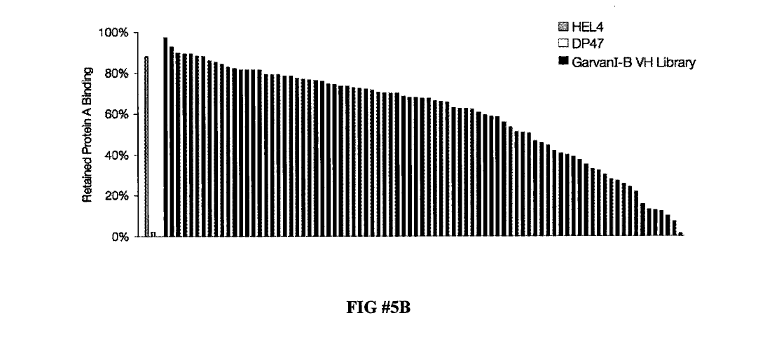

The results of these aggregation-resistance experiments are shown in FIGS. 5A and 5B. In summary, the majority of naïve (unselected) clones of the Garvan-IA or -IB V_{H} library, in which diversity was introduced into CDR3 of HEL4, exhibited a considerable level of aggregation-resistance when subjected to the “Heat/Cool” assay. Diversity was restricted to CDR3 only of the HEL4 scaffold at either 7 or 9 amino acid residues (IA and IB, respectively).

### Example 10

### Isolation and Characterization of G07 and G11 Clones

Two clones selected for their binding to human tumour necrosis factor (hTNF) or mouse interleukin 21 (mIL-21) were isolated from the Garvan-I libraries, as detailed above (see Materials and Methods section). Binding was antigen-specific, as demonstrated by ELISA. Biacore affinity measurements of anti-human TNF clone G07 (SEQ ID NO: 5) and anti-mouse IL-21 clone G11 (SEQ ID NO: 6) were performed. Serial dilutions of each purified protein, starting at 4 μM, were run on a streptavidin (SA) chip coated with biotinylated hTNF and mIL-21, respectively. Biacore software analysis estimated an affinity of 1.86 μM and 4.07 μM for G07 and G11, respectively.

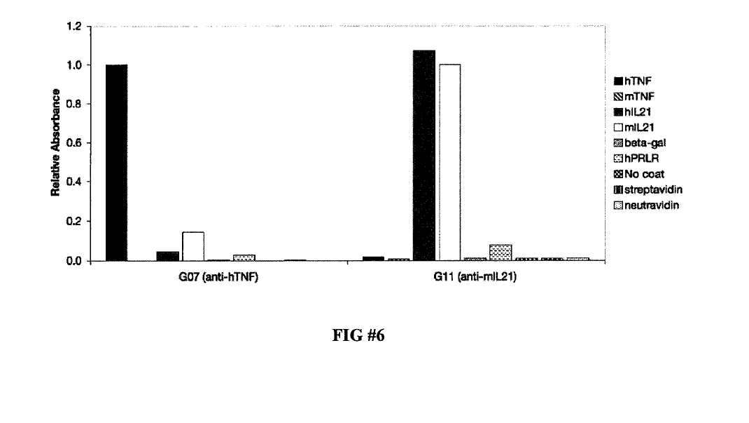

The specificity of antigen-binding was then investigated for each V_{H}. Purified, c-Myc tagged, antibody domains were tested for binding to hTNF, mIL-21 and a range of unrelated antigens immobilized on a Nunc Maxisorb ELISA plate. Bound antibody domain was detected using an anti-c-Myc antibody. The results are shown in FIG. 6. In summary, both clones bound specifically to their cognate antigens. G11 was also found to bind to human IL-21 (hIL-21), a homolog of mIL-21.

### Example 11

### Aggregation-Resistance of Antigen Binding Anti-hTNF Clone G07 and Anti-mIL-21 Clone G11

The aggregation-resistance of the antigen binding anti-hTNF clone G07 and anti-mIL-21 clone G11 are tested on phage. Also, the CDR3 of these clones is incorporated into one or more of the DP47-CDR1 mutants described herein above to investigate the effects of the point mutations on antigen binding and aggregation-resistance. The aggregation-resistance of mutant, antigen-binding V_{H} is measured using the phage “Heat/Cool” assay as described in the Materials and Methods section above. In addition, binding to antigen is tested by ELISA to evaluate the effect of CDR1 mutations on antigen binding.

### Example 12

### Identification of Additional Mutations that Confer Aggregation-Resistance

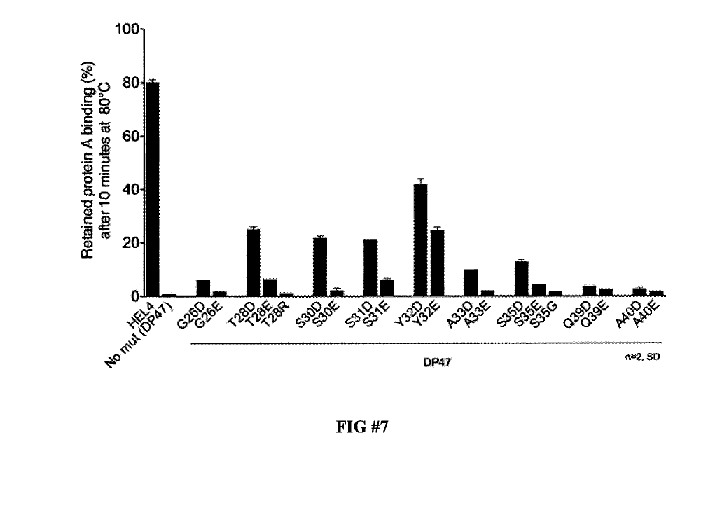

To identify additional residues in CDR1 that confer aggregation-resistance on V_{H}, surface-exposed residues of DP47 between positions 26 to 35 (numbering according to the Kabat numbering system) were substituted for aspartic acid (D) or glutamic acid (E). Framework residues at positions 39 and 40 were also substituted for aspartic acid (D) or glutamic acid (E). These mutant V_{H} were displayed on phage and subjected to the “Heat/Cool” assay, as described in Example 1.2. Results of this assay are shown in Table 4 and FIG. 7. Briefly, phage-displayed V_{H} were heated to 80° C. for 10 min, followed by cooling at 4° C. for 10 min. Correctly folded V_{H} were captured by protein A ELISA and the absorbance signal of the treated sample was calculated as a percentage of the untreated sample.

TABLE 4
Aggregation-resistance of V_{H} mutants. Retained protein
A binding (in %) of DP47 single mutants displayed on phage
after heating to 80° C. for 10 minutes, followed by 4° C.
for 10 minutes and captured by protein A.

| | Retained binding | Standard |
|---|---|---|
| | to protein A (%) | Deviation (n = 2) |
| HEL4 | 80.2 | 1.45 |
| DP47 | 1.1 | 0.08 |
| (no mutation) | | |
| G26D | 6.1 | 0.3 |
| G26E | 1.8 | 0.17 |
| T28D | 25.1 | 1.12 |
| T28E | 6.5 | 0.03 |
| S30D | 21.8 | 0.73 |
| S30E | 2.1 | 0.99 |
| S31D | 21.3 | 0.04 |
| S31E | 6.1 | 0.45 |
| Y32D | 41.8 | 2.28 |
| Y32E | 24.8 | 1.10 |
| A33D | 10.0 | 0.22 |
| A33E | 2.0 | 0.11 |
| S35D | 12.8 | 0.53 |
| S35E | 4.5 | 0.45 |
| Q39D | 3.7 | 0.14 |
| Q39E | 2.5 | 0.25 |
| A40D | 2.8 | 0.48 |
| A40E | 1.9 | 0.15 |

The data showed that substitutions at positions 28, 30, 31, 32, 33 or 35 considerably increased aggregation-resistance of DP47. Substitutions (aspartic acid or glutamic acid) at other positions (26, 39, and 40) also detectably increased aggregation-resistance of DP47.

The data also showed that aspartic acid substitutions increased aggregation-resistance more than glutamic acid substitutions. This effect was observed at each of the positions described above.

### Example 13

### Identification of Mutations in CDR2 that Confer Aggregation-Resistance

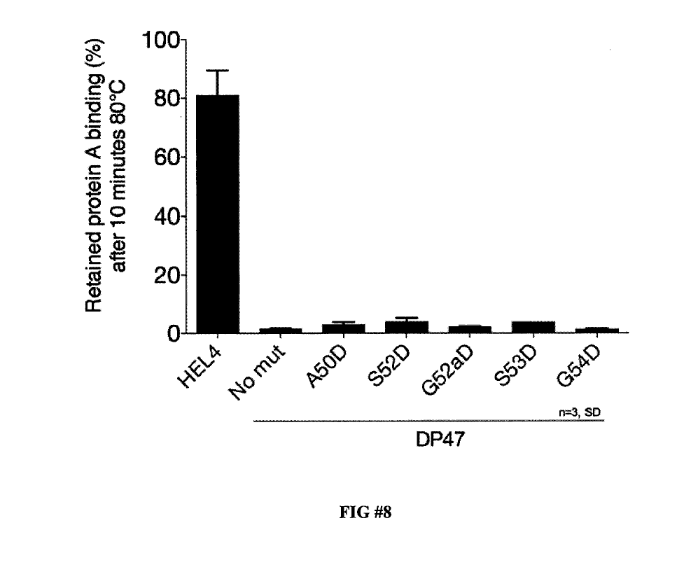

To identify residues in CDR2 that confer aggregation-resistance on a V_{H} containing protein aspartic acid (D) was substituted for surface-exposed residues within the putative CDR2 of DP47 V_{H} with aspartic acid (positions 50, 52, 52a, 53, 54). These mutant V_{H} were displayed on phage and subjected to the “Heat/Cool” assay, as described in Example 1.2. Results of this assay are shown in Table 5 and FIG. 8.

TABLE 5
Aggregation-resistance of V_{H} mutants. Retained protein A
binding (in %) of DP47 single mutants displayed on
phage after heating to 80° C. for 10 minutes, followed by 4° C.
for 10 minutes and captured by protein A.

| | Retained binding to | Standard |
|---|---|---|
| | protein A (%) | Deviation (n = 3) |
| HEL4 | 80.8 | 8.6 |
| DP47 (no mutation) | 1.4 | 0.3 |
| A50D | 2.9 | 0.9 |
| S52D | 3.9 | 1.3 |
| G52aD | 2.1 | 0.3 |
| S53D | 3.8 | 0.0 |
| G54D | 1.3 | 0.2 |

These data showed that introduction of a negatively charged amino acid at position 50, 52, 52a or 53 of DP47 V_{H} detectably increased aggregation-resistance.

### Example 14

### Effect of Different Combinations of Negatively Charged Amino Acids at Positions 31 and/or 32 and/or 33

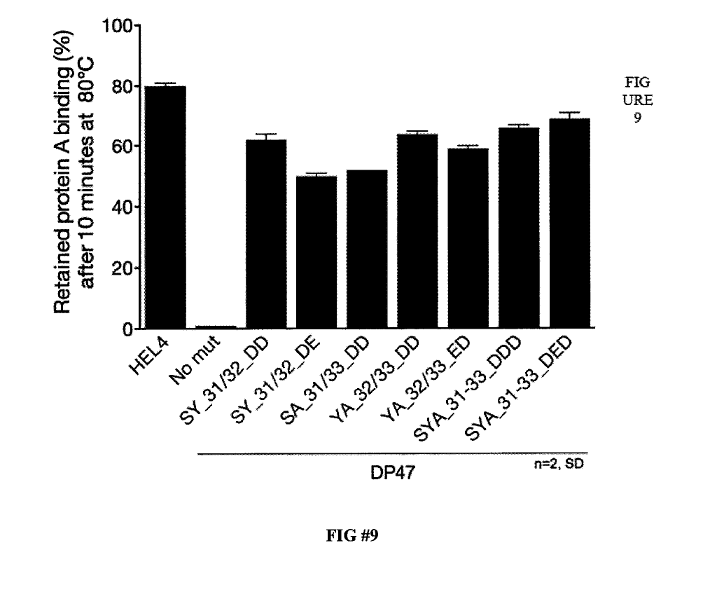

V_{H} domains were produced comprising different combinations of negatively charged amino acids at positions 31 and/or 32 and/or 33 as set out in Table 6. Aggregation-resistance of the V_{H} domains was assessed using the “Heat/Cool” assay as described in Example 1.2. Results are shown in Table 6 and FIG. 9.

TABLE 6
Aggregation-resistance of double or
triple negative-charge mutants of
DP47 at positions 31 and/or 32 and/or 33, as
determined by the heat/cool assay on phage.

| | Retained binding to | Standard |
|---|---|---|
| | protein A (%) | Deviation (n = 2) |
| HEL4 | 80 | 1 |
| DP47 (no mut) | 1 | 0 |
| SY_31/32_DD | 62 | 2 |
| SY_31/32-DE | 50 | 1 |
| SA_31/33_DD | 52 | 0 |
| YA_32/33_DD | 64 | 1 |
| YA_32/33_ED | 59 | 1 |
| SYA_31-33_DDD | 66 | 1 |
| SYA_31-33_DED | 69 | 2 |

These data demonstrate that all combinations of negatively charged amino acids tested confer a considerable degree of aggregation-resistance on V_{H} domains. The data also show that combinations containing only aspartic acid as negatively charged amino acids generally confer a greater degree of aggregation-resistance than combinations comprising glutamic acid. Furthermore, these data demonstrate that multiple negatively charged amino acids confer a higher degree of aggregation-resistance than is observed for any single negatively charged amino acid (see FIG. 7 and Table 4).

### Example 15

### Combinations of Mutations at Positions 28 and/or 35 and Other Sites in CDR1 Confer Aggregation-Resistance on a V_{H} Containing Protein

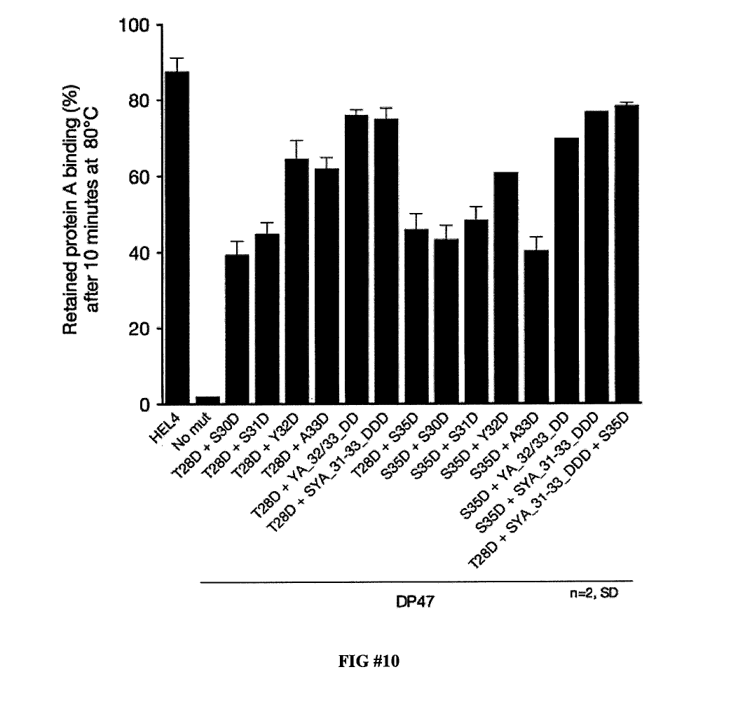

V_{H} domains were produced comprising negatively charged amino acids at positions 28 and/or 35 in CDR1 of DP47 and at least one other position, as set out in Table 7. Aggregation-resistance of the V_{H} domains was assessed using the “Heat/Cool” assay as described in Example 1.2. Results are shown in Table 7 and FIG. 10.

TABLE 7
Aggregation-resistance of V_{H} domains
comprising negatively charged
amino acids at least at position 28 and/or 35.

| | Retained | |
|---|---|---|
| | binding to | Standard |
| | protein | Deviation |
| | A (%) | (n = 2) |
| HEL4 | 87.7 | 3.5 |
| DP47 (no mut) | 1.8 | 0.3 |
| T28D + S30D | 39.4 | 3.9 |
| T28D + S31D | 44.8 | 2.8 |
| T28D + Y32D | 64.7 | 4.8 |
| T28D + A33D | 62.0 | 2.3 |
| T28D + YA_32/33_DD | 76.1 | 1.2 |
| T28D + SYA_31-33_DDD | 74.9 | 2.4 |
| T28D + S35D | 45.8 | 3.9 |
| S35D + S30D | 43.5 | 2.9 |
| S35D + S31D | 48.3 | 3.2 |
| S35D + Y32D | 61.0 | 0.4 |
| S35D + A33D | 40.3 | 3.8 |
| S35D + YA_32/33_DD | 70.4 | |
| S35D + SYA_31-33_DDD | 76.7 | 0.0 |
| T28D + SYA_31-33_DDD + S35D | 78.6 | 0.3 |

These data demonstrate that combining negatively charged amino acids at position 28 or 35 with additional negatively charged amino acids confers a higher degree of aggregation-resistance than is observed for single negatively charged amino acid at these positions.

### Example 16

### Negatively Charged Amino Acids in CDR1 Confer High Levels of Expression of Soluble Protein

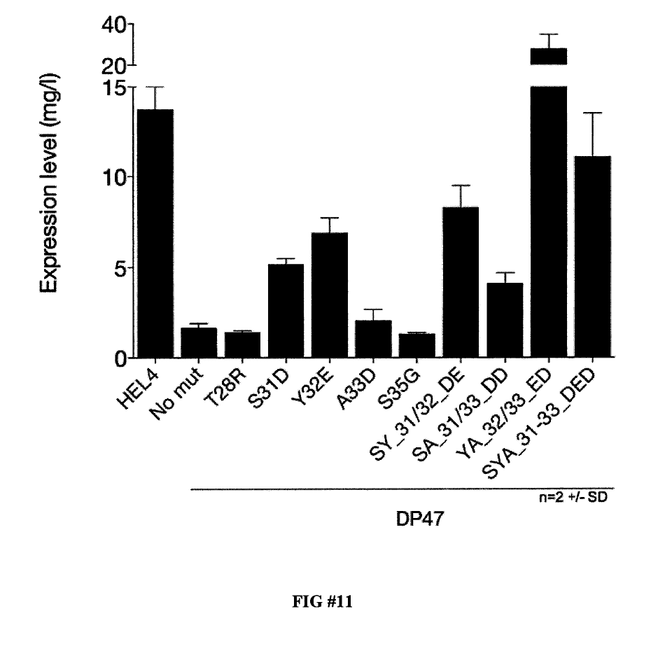

Soluble expression levels of V_{H} domains were assessed using a process as described in Example 1.7. V_{H} domains studied included DP47, HEL4 and combinations of negatively charged amino acids in CDR1 of DP47. Results are shown in Table 8 and FIG. 11.

TABLE 8
Soluble expression levels (mg/l) of
DP47-CDR1 V_{H} mutants after 42 hr
induction at 30° C., measured by protein A ELISA.

| | Soluble Expression (mg/l) | | | |
|---|---|---|---|---|
| | #1 | #2 | Mean | St Dev |
| HEL4 | 15.0 | 12.4 | 13.7 | 1.8 |
| DP47 (no mutations) | 1.4 | 1.9 | 1.7 | 0.4 |
| T28R | 1.5 | 1.3 | 1.4 | 0.1 |
| S31D | 4.8 | 5.5 | 5.2 | 0.5 |
| Y32E | 7.7 | 6.0 | 6.9 | 1.2 |
| A33D | 1.4 | 2.7 | 2.1 | 0.9 |
| S35G | 1.2 | 1.4 | 1.3 | 0.1 |
| SY_31/32_DE | 7.0 | 9.5 | 8.3 | 1.8 |
| SA_31/33_DD | 3.5 | 4.7 | 4.1 | 0.8 |
| YA_32/33_ED | 20.4 | 34.9 | 27.7 | 10.3 |
| SYA_31-33_DED | 13.5 | 8.6 | 11.1 | 3.5 |

Expression levels were considerably increased for mutants of DP47 containing two or more negative charged residues at positions 31 to 33. Single mutations also showed a modest improvement over DP47 expression levels, but less so than for double or triple mutations.

### Example 17

### Aggregation-Resistance of CDR1 Mutants as Soluble V_{H} Protein, Measured by Size Exclusion Chromatography

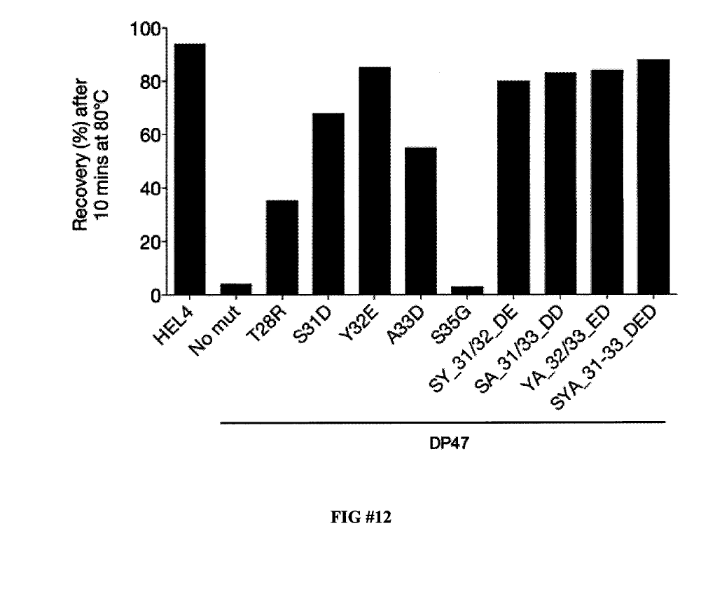

Aggregation-resistance of purified V_{H} domains was studied using size exclusion chromatography. V_{H} domains studied included DP47, HEL4 and combinations of negatively charged amino acids in CDR1 of DP47. Results are shown in Table 9 and FIG. 12.

TABLE 9
Recovery of 10 μM solution of soluble DP47 V_{H}
with CDR1 mutations, after heating for 10 mins
at 80° C., as measured by eluted protein
on Superdex-G75 size-exclusion column.

| | Recovery after |
|---|---|
| | heating (%) |
| HEL4 | 93.7 |
| DP47 (no mut) | 3.4 |
| T28R | 34.7 |
| S31D | 69.4 |
| Y32E | 85.7 |
| A33D | 55.8 |
| S35G | 2.2 |
| SY_31/32_DE | 80.6 |
| SA_31/33_DD | 83.9 |
| YA_32/33_ED | 83.7 |
| SYA_31-33_DED | 87.6 |

These data demonstrate that negatively charged amino acid(s) at positions 31 and/or 32 and/or 33 increases aggregation-resistance of human V_{H} domains, particularly for those variants containing two or more substitution.

### Example 19

### Aggregation-Resistance of CDR1 Mutants as Soluble Protein, Measured by Circular Dichroism

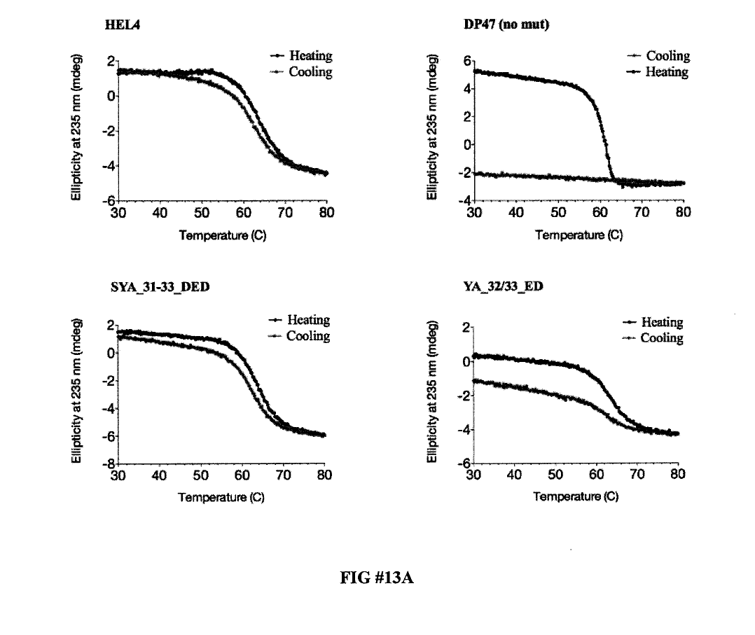

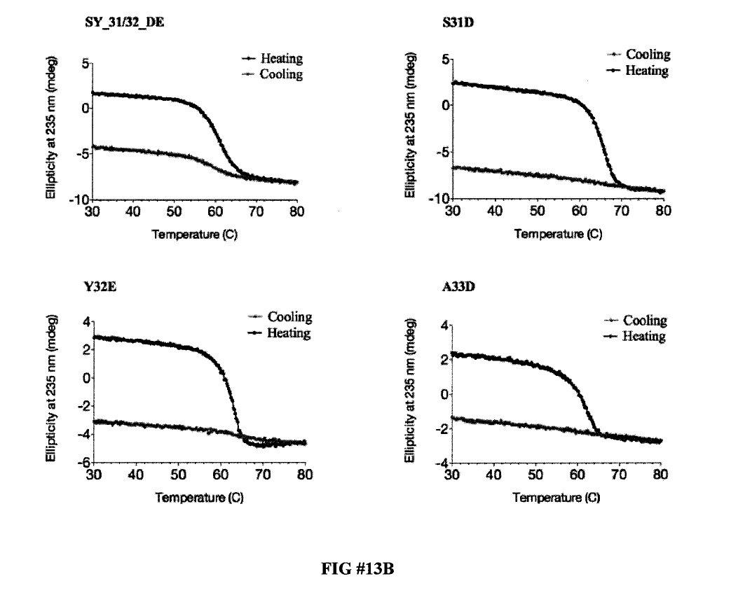

Aggregation-resistance of purified V_{H} domains was studied using CD. V_{H} domains studied included DP47, HEL4 and combinations of negatively charged amino acids in CDR1 of DP47. Results are shown in FIGS. 13A and B.

The melting curves were consistent with two-state transitions for each protein. HEL4 exhibited full aggregation-resistance after heating to 80° C., whereas DP47 aggregated upon heat denaturation and was unable to refold. The single negative charge mutations (S31D, Y32E, A33D) showed negligible signs of refolding under these conditions. Thus, only the introduction of double negative substitutions at positions 31 and/or 32 and/or 33 detectably improved the aggregation-resistance of the domains, with a triple mutation at positions 31, 32 and 33 providing the largest effect. This demonstrates that multiple negative-charge substitutions confer aggregation-resistance in solution.

### Example 19

### CDR1 Mutations Reduce Retention of Soluble V_{H} in Size-Exclusion Columns

Retention of V_{H} domains in size exclusion columns was analyzed. V_{H} domains studied included DP47, HEL4 and various combinations of negatively charged amino acids in CDR1. Results are shown in Table 10.

TABLE 10
Monomer elution volume of soluble DP47
V_{H} with CDR1 mutations from
Superdex-G75 size-exclusion column.

| | Monomer elution |
|---|---|
| | volume (ml) |
| HEL4 | 15.7 |
| DP47 (no mut) | 24.9 |
| T28R | 25.8 |
| S31D | 23.2 |
| Y32E | 23.4 |
| A33D | 22.5 |
| S35G | 22.9 |
| SY_31/32_DE | 22.0 |
| SA_31/33_DD | 21.2 |
| YA_32/33_ED | 20.7 |
| SYA_31-33_DED | 19.7 |

It was observed that the elution volumes of variants with multiple negative-charge substitutions were considerably lower than that of DP47 or single mutants. These data demonstrate that multiple negative-charge substitutions at 31, 32 and/or 33 reduce retention of human V_{H} domains in size exclusion columns. This provides an advantage in so far as it facilitates purification of proteins comprising such V_{H} domains. Moreover, the aggregation-resistance of these V_{H} domains also means that proteins containing the domains can be heated to reduce the prevalence of aggregates and/or dimers/trimers and then separated using size exclusion chromatography to thereby produce a purified protein. Such a method facilitates greater recovery of useful product.

### Example 20

### Aggregation-Resistance of Antigen-Specific V_{H} Domains

Antigen-specific V_{H} domains were selected from the Garvan-2 library by phage display against recombinant protein antigen. After selection, the domains were evaluated for aggregation-resistance using the “Heat/Cool” assay described in Example 1.2, and binding to either or protein A superantigen and or recombinant antigen. Results are shown in Table 11.

TABLE 11
Aggregation-resistance of antigen-specific V_{H} domains
as determined by “Heat/Cool” assay on phage.

| | | Retained | Standard |
|---|---|---|---|
| | Superantigen | binding to | Deviation |
| | Antigen | protein A (%) | (n = 2) |
| HEL4 | Protein A | 88.1 | 4.2 |
| DP47 | Protein A | 1.4 | 0.1 |
| V_{H}PRLR_C02 | Protein A | 98.8 | 2.9 |
| V_{H}PRLR_C02 | hPRLR | 63.8 | 19.0 |
| V_{H}HEL_H04 | Protein A | 87.8 | 2.3 |
| V_{H}HEL_H04 | HEL | 61.0 | 5.0 |
| V_{H}HEL_G08 | Protein A | 83.8 | 0.6 |
| V_{H}HEL_G08 | HEL | 68.1 | 5.1 |

This analysis revealed that the selected antigen-specific V_{H} domains (V_{H}PRLR_C02: anti-PRLR (Prolactin Receptor; SEQ ID NO: 7); V_{H}HEL_H04: anti-Hen-Egg-Lysozyme, SEQ ID NO: 8; V_{H}HEL_H08: anti-Hen-Egg-Lysozyme, SEQ ID NO: 9) displayed considerable aggregation-resistance on phage (Table 11). All of the selected binders contained two or more negatively charged amino acids at positions 31 and/or 32 and/or 33.

The antigen-specific V_{H} domains were expressed and purified. Affinity measurements were performed by surface plasmon resonance on a Biacore 2000 instrument. This revealed high affinity binding to the target antigen (Table 12).

TABLE 12
Affinity of purified
antigen-specific V_{H} domains

| | Affinity (K_{D}) | |
|---|---|---|
| V_{H}PRLR_C02 | <100 | nM |
| V_{H}HEL_H04 | 28 | nM |
| V_{H}HEL_G08 | 65 | nM |

The purified proteins were also analysed for aggregation-resistance. For this analysis the purified V_{H} domain-containing samples at 10 μM in PBS were either heated to 80° C. for 10 mins followed by cooling at 4° C. for 10 mins or not treated. Both heated and unheated samples were centrifuged at 16,000×g for 10 mins before 500 μl of each were analysed on a Superdex-G75 column (Pharmacia) equilibrated with 25 mM sodium phosphate (pH 7.4) containing 125 mM NaCl. The proteins were injected at a volume of 500 μl with a flow rate of 0.5 ml/min. The recovery of each V_{H} mutant was determined by measuring the area under the curve of the heated sample, expressed as percentage of the unheated sample. This revealed that the purified antigen-specific V_{H} domains displayed considerable aggregation resistance (Table 13).

All oft the antigen-specific V_{H} domains contained two or more negatively charged amino acids at positions 31 and/or 32 and/or 33.

TABLE 13
Aggregation-resistance of purified
antigen-specific V_{H} domains

| | Recovery after |
|---|---|
| | heating (%) |
| V_{H}PRLR_C02 | 92 |
| V_{H}HEL_H04 | 102 |
| V_{H}HEL_G08 | 91 |

### REFERENCES

Al-Lazikani et al., J Mol Biol 273, 927-948, 1997;
Andersson-Engels et al, Phys. Med. Biol, 42:815-824, 1997;
Arbabi-Ghahroudi et al., Prot. Eng., Des. & Sel., 22: 59-66, 2009;
F. M. Ausubel et al. (editors), Current Protocols in Molecular Biology, Greene Pub. Associates and Wiley-Interscience (1988, including all updates until present);
Bendele J Musculoskel Neuron Interact; 1(4):377-385, 2001;
Borrebaeck (ed), Antibody Engineering, Oxford University Press, 1995 (ISBN0195091507);
Bork et al., J Mol. Biol. 242, 309-320, 1994;
Bradl and Linington Brain Pathol., 6:303-311, 1996;
Brennan et al, Science, 229: 81-83, 1985;
Brinkmann et al., Proc. Natl. Acad. Sci. USA, 90: 7538-7542, 1993;
Carter et al Nucleic Acids Res. 13:4431-4443, 1985;
Carter et al. Bio/Technology 10: 163-167, 1992;
Chen et al. Nature, 446:203-207, 2007;
Cheung et al., Virology 176:546, 1990;
Chothia and Lesk J. Mol Biol. 196:901-917, 1987;
Chothia et al. Nature 342, 877-883, 1989;
Dooley and Flajnik, Dev Comp Immunol. 30:43-56, 2006;
Ewert et al., J. mol. Biol., 325: 531-553, 2003;
Frangioni, Curr. Opin. Chem. Biol, 7:626-634, 2003;
Goding, Monoclonal Antibodies: Principles and Practice, Academic Press, (1986) pp. 59-103;
Goodman et al., (editors) Goodman and Gilman's The Pharmacological Basis of Therapeutics, 8^{th} Ed., Macmillan Publishing Co. (1990);
Guss et al. EMBO J. 5: 1567-1575, 1986;
Guy et al., Mol Cell Biol. 12(3):954-61, 1992;
Harlow and Lane, Antibodies: A Laboratory Manual, Cold Spring Harbor Press, 1988;
Harris et al., Trends Biotechnol., 17: 290-296, 1999;
Higuchi et al., Nucleic Acids Res 16(15): 7351-7367, 1988;
Higuchi, in PCR Protocols, pp. 177-183, Academic Press, 1990;
Ho et al Gene (Amst.) 77:51-59, 1989;
Holliger et al Proc. Natl. Acad Sci. USA 90: 6444-6448, 1993;
Hollinger and Hudson Nature Biotechnology, 23: 1126-1136, 2005;
Hoogenboom and Winter J Mol Biol, 227:381, 1991;
Hoyer et al., Biophys. Chem., 96: 273-284, 2002;
Hu et al., Cancer Res., 56: 3055-3061, 1996;
Hudson and Kortt J. Immunol. Methods, 231: 177-189, 1999;
Hust et al., BMC Biotechnology 7:14, 2007;
Ito et al Gene 102:67-70, 1991;
Jakobovits et al. Nature Biotechnology 25, 1134-1143, 2007;
Jespers et al., J Mol Biol.; 337: 893-903, 2004;
Kabat Sequences of Proteins of Immunological Interest, National Institutes of Health, Bethesda, Md., 1987 and 1991;
Kabat, E., Wu, T. T., Perry, H. M., Kay, S. and Gottesman, C. F. (1992) Sequences of Proteins of Immunological Interest. 5 ed. DIANE Publishing;
Kirkland et al., J. Immunol. 137:3614, 1986;
Kohler and Milstein Nature, 256:495-497, 1975;
Kostelny et al, J. Immunol., 148(5):1547-1553, 1992;
Kruif and Logtenberg J. Biol. Chem., 271: 7630-7634, 1996;
Kunkel et al., Methods Enzymol., 154: 367, 1987;
Lee et al., Nat Protoc., 2: 3001-3008, 2007;
Levin and Weiss, Mol Biosyst., 2: 49-57, 2006;
Lonberg, N. “Transgenic Approaches to Human Monoclonal Antibodies.” Handbook of Experimental Pharmacology 113: 49-101, 1994;
Largaespada et al, Curr. Top. Microbiol. Immunol, 166, 91-96, 1990;
Lindmark et al. J Immunol Meth. 62: 1-13, 1983;
Marks et al, J. Mol. Biol., 222:581-597, 1991;
Matsui et al., Cell. 61(6):1147-55, 1990;
Matusik et al., In: Transgenics in Endocrinology, ed. By M M Matzuk, C W Brown, and T R Kumar. The Humana Press Inc (Totowa, N.J.) Chapter 19, pp 401-425, 2001
McCafferty et al., Nature, 348: 552-554, 1990;
Moldenhauer et al., Scand. J. Immunol. 32:77, 1990;
Muller et al EMBO J. 9(3):907-13, 1990;
Plückthun, Immunol. Revs., 130:151-188, 1992;
Plückthun in The Pharmacology of Monoclonal Antibodies, vol. 113, Rosenburg and Moore eds., Springer Verlag, New York, pp. 269-315, 1994;
Presta et al., Cancer Res., 57: 4593-4599, 1997;
Ramanujam et al, IEEE Transactions on Biomedical Engineering, 48:1034-1041, 2001;
Risma et al., Proc Natl Acad Sci USA.; 92(5):1322-6, 1995;
Roby et al., Carcinogenesis. 21(4):585-91, 2000;
Roux et al. J. Immunol. 161:4083, 1998;
Saha et al., BcePred:Prediction of Continuous B-Cell Epitopes in Antigenic Sequences Using Physico-chemical Properties. In Nicosia, Cutello, Bentley and Timis (Eds.) ICARIS 2004, LNCS 3239, 197-204, Springer, 2004;
Sakaguchi et al. Nature, 426: 454-460;
Sambrook et al. Molecular Cloning: A Laboratory Manual, Cold Spring Harbour Laboratory Press, 1989;
Sanchez-Ruiz, et al., Biochemistry, 27: 1648-52, 1988
Scopes In: Protein purification: principles and practice, Third Edition, Springer Verlag, 1994;
Skerra et al, Curr. Opinion in Immunol., 5:256-262, 1993;
Stahli et al., Methods in Enzymology 9:242, 1983;
Strenglin et al EMBO J, 7, 1053-1059, 1988;
Tang et al. J. Exp. Med., 199: 1455-1465, 2004;
Trenado et al. J. Clin. Invest., 112: 1688-1696, 2002;
Van der Sluis et al. Gastroenterology 131: 117-129, 2006;
van Mierlo and Steemsma, J. Biotechnol, 79:281-98, 2000
Wang et al. J Clin Invest. 118(7): 2629-2639, 2008;
Weissinger et al. Proc. Natl. Acad. Sci USA, 88, 8735-8739, 1991;
Wells et al Gene 34:315-323, 1985;
Willuda et al., Cancer Res., 59: 5758-5767, 1999; and
Zoller and Smith, Methods Enzymol., 154: 329, 1987.

1. An isolated protein comprising:
a) an antibody heavy chain variable region (V_{H}) comprising a negatively charged amino acid at two or more positions selected from the group consisting of 28 and/or 31 and/or 32 and/or 33 and/or 35 according to the numbering system of Kabat, the protein capable of specifically binding to an antigen other than hen egg lysozyme, beta galactosidase, alpha amylase, B5R or wherein:
(i) if the protein binds to human vascular endothelial growth factor (VEGF) and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35; or
b) an antibody heavy chain variable region (V_{H}) comprising two or more negatively charged amino acid at positions selected from the group consisting of 28 and/or 31 and/or 32 and/or 33 and/or 35 according to the numbering system of Kabat, the protein capable of specifically binding to an antigen with an affinity of more than 10 μM, wherein:
(i) if the protein binds to human vascular endothelial growth factor (VEGF) and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35; or
c) an antibody heavy chain variable region (V_{H}) comprising a negatively charged amino acid at position 28, 33 and/or 35 according to the numbering system of Kabat, the protein capable of specifically binding to an antigen other than hen egg lysozyme, beta galactosidase, alpha amylase B5R or wherein:
(i) if the protein binds to human vascular endothelial growth factor (VEGF) and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35; or
d) an antibody heavy chain variable region (V_{H}) comprising a negatively charged amino acid at position 28, 33 and/or 35 according to the numbering system of Kabat, the protein capable of specifically binding to an antigen with an affinity of more than 10 μM, wherein:
(i) if the protein binds to human vascular endothelial growth factor (VEGF) and comprises aspartic acid at positions 32 and 33 it comprises at least one additional negatively charged amino acid between positions 29 and 35; and
(ii) if the protein binds to human VEGF and comprises aspartic acid at positions 31 and 33 it comprises at least one additional negatively charged amino acid between positions 28 and 35.

2. (canceled)

3. (canceled)

4. (canceled)

5. The isolated protein of claim 1, part b), wherein the protein is capable of specifically binding to an antigen with an affinity of more than 100 nM.

6. The protein of claim 1, part a), having reduced tendency to aggregate compared to the protein without the negatively charged amino acid(s) at position 28 and/or 31 and/or 32 and/or 33 and/or 35 according to the numbering system of Kabat.

7. The protein of claim 1, part a), having reduced tendency to aggregate after heating to at least about 60° C. compared to the protein without the negatively charged amino acid(s) at position 28 and/or 31 and/or 32 and/or 33 and/or 35 according to the numbering system of Kabat.

8. The protein according to claim 1, part a), having an ability to specifically bind to the antigen after heating to at least about 60° C.

9. The protein of claim 7, having reduced tendency to aggregate and/or an ability to specifically bind to the antigen after heating to at least about 80° C.

10. The protein of claim 1, part a), capable of binding to a human protein.

11. The protein of claim 1, part a), capable of binding to a protein associated with or causative of a human condition.

12. The protein of claim 1, part a), wherein the negatively charged amino acid at position 32 is glutamic acid.

13. The protein of claim 1, part a), wherein the negatively charged amino acid is aspartic acid.

14. The protein of claim 1, part a), additionally comprising a negatively charged amino acid at one or more residues selected individually or collectively from the group consisting of position 26, 30, 39, 40, 50, 52, 52a and 53 according to the numbering system of Kabat.

15. The protein of claim 14, wherein the negatively charged amino acid is aspartic acid.

16. The protein of claim 1, part a), comprising negatively charged amino acids at positions 31 and 32 and 33 according to the numbering system of Kabat.

17. The protein of claim 16 comprising:
(i) an aspartic acid at position 31 according to the numbering system of Kabat;
(ii) a glutamic acid or aspartic acid at position 32 according to the numbering system of Kabat; and
(iii) an aspartic acid at position 33 according to the numbering system of Kabat.

18. The protein of claim 1, part a), comprising negatively charged amino acids at positions 32 and 33 according to the numbering system of Kabat.

19. The protein of claim 18 comprising:
(i) a glutamic acid or aspartic acid at position 32 according to the numbering system of Kabat; and
(ii) an aspartic acid at position 33 according to the numbering system of Kabat.

20. The protein of claim 16 additionally comprising a negatively charged amino acid at position 28 and/or 35.

21. The protein of claim 20, wherein the negatively charged amino acid at position 28 and/or 35 is aspartic acid.

22. The protein of claim 1, part a), which is human, humanized or deimmunized at amino acid positions other than position 28 and/or 31 and/or 32 and/or 33 and/or 35 according to the numbering system of Kabat or is fused to a human protein or region thereof.

23. A protein comprising:
a) a modified antibody heavy chain variable region (V_{H}) capable of specifically binding to an antigen, wherein the V_{H} comprises a negatively charged amino acid at position 28, 31, 33 and/or 35 according to the numbering system of Kabat, and wherein the unmodified form of the V_{H} does not comprise the negatively charged amino acid(s); or
b) a modified antibody heavy chain variable region (V_{H}) capable of specifically binding to an antigen, wherein the V_{H} comprises negatively charged amino acids at two or more positions selected from the group consisting of 28 and/or 31 and/or 32 and/or 33 and/or 35 according to the numbering system of Kabat, and wherein the unmodified protein does not comprise the two or more negatively charged amino acids at positions 28 and/or 31 and/or 32 and/or 33 and/or 35 according to the numbering system of Kabat.

24. (canceled)

25. The protein according to claim 23, part a), comprising:
(i) an aspartic acid at position 31 according to the numbering system of Kabat; and/or
(ii) a glutamic acid at position 32 according to the numbering system of Kabat; and/or
(iii) an aspartic acid at position 33 according to the numbering system of Kabat.

26. The protein of claim 25 additionally comprising a negatively charged amino acid at position 28 and/or 35.

27. The protein of claim 26, wherein the negatively charged amino acid at position 28 and/or 35 is aspartic acid.

28. The protein of claim 1, part a), wherein the protein is selected from the group consisting of:
(i) an antibody;
(ii) a single domain antibody
(iii) a single chain Fv (scFv) containing protein
(iv) a diabody, a triabody or a tetrabody; and
(v) a fusion protein comprising any one of (ii)-(iv) and a Fc domain of an antibody or a domain thereof.

29. The protein according to claim 1, part a), conjugated to a compound.

30. The protein according to claim 29, wherein the compound is selected from the group consisting of a radioisotope, a detectable label, a therapeutic compound, a colloid, a toxin, a nucleic acid, a peptide, a protein, a compound that increases the half life of the protein in a subject and mixtures thereof.

31. A composition comprising the protein of claim 1, part a) and a pharmaceutically acceptable carrier.

32. A library comprising:
a) a plurality of proteins according to claim 1, part a); or
b) proteins comprising antibody heavy chain variable regions (V_{H}s), wherein at least 30% of the V_{H}s comprise negatively charged amino acids at two or more positions selected from the group consisting of 28 and/or 31 and/or 32 and/or 33 and/or 35 according to the numbering system of Kabat.

33. (canceled)

34. A method for isolating the protein of claim 1, part a), the method comprising contacting the library of claim 32 with the antigen and isolating a protein that binds thereto.

35. A method for increasing the aggregation-resistance of a protein comprising an antibody heavy chain variable region (V_{H}), the method comprising:
a) modifying the V_{H} by substituting an amino acid at position 28, 31, 33 and/or 35 according to the numbering system of Kabat with a negatively charged amino acid; or
b) modifying the V_{H} by substituting two or more amino acids at position 28 and/or 31 and/or 32 and/or 33 and/or 35 according to the numbering system of Kabat with a negatively charged amino acid, wherein the unmodified protein does not comprise negatively charged amino acids at the substituted position(s); or
c) modifying the V_{H} such that it comprises negatively charged amino acids at two or more positions selected from the group consisting of 28 and/or 31 and/or 32 and/or 33 and/or 35 according to the numbering system of Kabat, wherein the unmodified protein does not comprise the two or more negatively charged amino acids at positions 28 and/or 31 and/or 32 and/or 33 and/or 35 according to the numbering system of Kabat.

36. (canceled)

37. (canceled)

38. A method for:
a) increasing the level of production of a soluble protein comprising an antibody heavy chain variable region (V_{H}), the method comprising modifying the V_{H} by substituting two or more amino acids at position 28 and/or 31 and/or 32 and/or 33 and/or 35 according to the numbering system of Kabat with a negatively charged amino acid, wherein the level of soluble protein produced is increased compared to the level of production of protein lacking the negatively charged amino acids; or
b) increasing the level of production of a soluble protein comprising an antibody heavy chain variable region (V_{H}), the method comprising modifying the V_{H} by substituting an amino acid at position 28 and/or 31 and/or 33 and/or 35 according to the numbering system of Kabat with a negatively charged amino acid and producing the protein, wherein the level of soluble protein produced is increased compared to the level of production of protein lacking the negatively charged amino acids; or
c) increasing the level of recovery of a protein comprising an antibody heavy chain variable region (V_{H}) from a chromatography resin or for reducing volume of solution required to recover the protein from a chromatography resin, the method comprising modifying the V_{H} by substituting two or more amino acids at position 28 and/or 31 and/or 32 and/or 33 and/or 35 according to the numbering system of Kabat with a negatively charged amino acid and contacting the protein with a chromatography resin, wherein the level of recovery of the protein recovered from a chromatography resin is increased or the volume of solution required to recover the protein from a chromatography resin is reduced compared to a protein lacking the negatively charged amino acids; or
d) increasing the level of recovery of a protein comprising an antibody heavy chain variable region (V_{H}) from a chromatography resin or for reducing volume of solution required to recover the protein from a chromatography resin, the method comprising modifying the V_{H} by substituting an amino acid at position 28, 31, 33 and/or 35 according to the numbering system of Kabat with a negatively charged amino acid and contacting the protein with a chromatography resin, wherein the level of recovery of the protein recovered from a chromatography resin is increased or the volume of solution required to recover the protein from a chromatography resin is reduced compared to a protein lacking the negatively charged amino acids.

39. (canceled)

40. (canceled)

41. (canceled)

42. Use of the protein of claim 1, part a) in medicine.

43. A method of:
a) treating or preventing a condition in a subject, the method comprising administering the protein of claim 1, part a) to a subject in need thereof; or
b) delivering a compound to a cell, the method comprising contacting the cell with the protein of claim 1, part a); or
c) diagnosing or prognosing a condition in a subject, the method comprising contacting a sample from the subject with the protein of claim 1, part a) such that the protein binds to an antigen and form a complex and detecting the complex, wherein detection of the complex is diagnostic or prognostic of the condition in the subject.

44. (canceled)

45. (canceled)

46. The method of claim 43, part c), comprising determining the level of the complex, wherein an enhanced or reduced level of said complex is diagnostic or prognostic of the condition in the subject.

47. A method for localising or detecting an antigen in a subject, said method comprising:
(i) administering to a subject the protein of claim 29 such that the protein to binds to an antigen, wherein the protein is conjugated to a detectable label; and
(ii) detecting or localising the detectable label in vivo.
